(19) United States
(12) Patent Application Publication
(10) Pub. No.: US 2007/0093515 A1
(43) Pub. Date: Apr. 26, 2007

(54) Phosphodiesterase 10 inhibitors

(76) Inventors: Mark Phillip Arrington, Westwood, NJ (US); Richard D. Conticello, Ossining, NY (US); Carla Maria Gauss, White Plains, NY (US); Stephen Hitchcock, Westlake Village, CA (US); Allen Hopper, Montvale, NJ (US); Ruiping Liu, Huntington, NY (US); Truc Minh Nguyen, New York, NY (US); Ashok Tehim, Ridgewood, NJ (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/504,772

(22) Filed: Aug. 16, 2006

Related U.S. Application Data
(60) Provisional application No. 60/708,365, filed on Aug. 16, 2005.
(60) Provisional application No. 60/719,166, filed on Sep. 22, 2005.

(51) Int. Cl. A61K 31/517; C07D 403/02

(52) U.S. Cl. 514/266.21; 544/284; 514/234.2; 544/118; 514/252.17

(57) ABSTRACT

Provided are certain quinazolines that are PDE10 inhibitors, pharmaceutical compositions, containing the same and processes for preparing the same. Also provided are methods of treating diseases treatable by PDE10 enzyme such as obesity, non-insulin dependent diabetes, schizophrenia or bipolar disorder, obsessive-compulsive disorder, and the like, by administering those certain quinazolines.

This application claims the benefit of U.S. Patent Application No. 60/708,365, filed Aug. 16, 2005 and U.S. Patent Application No. 60/719,166, filed Sep. 22, 2005. The entire disclosure of each of those applications is hereby incorporated by reference.

Provided are certain quinazolines that are PDE10 inhibitors, pharmaceutical compositions containing such quinazolines and processes for preparing such quinazolines. Also provided are methods of treating diseases treatable by inhibition of PDE10 enzyme, such as obesity, non-insulin dependent diabetes, schizophrenia, bipolar disorder, obsessive-compulsive disorder, and the like, by administering such certain quinazolines.

Neurotransmitters and hormones, as well as other types of extracellular signals such as light and odors, create intracellular signals by altering the amounts of cyclic nucleotide monophosphates (cAMP and cGMP) within cells. These intracellular messengers alter the functions of many intracellular proteins. Cyclic AMP regulates the activity of cAMP-dependent protein kinase (PKA). PKA phosphorylates and regulates the function of many types of proteins, including ion channels, enzymes, and transcription factors. Downstream mediators of cGMP signaling also include kinases and ion channels. In addition to actions mediated by kinases, cAMP and cGMP bind directly to some cell proteins and directly regulate their activity.

Cyclic nucleotides are produced from the actions of adenylyl cyclase and guanylyl cyclase which convert ATP to cAMP and GTP to cGMP. Extracellular signals, often through the actions of G protein-coupled receptors, regulate the activity of the cyclases. Alternatively, the amount of cAMP and cGMP may be altered by regulating the activity of the enzymes that degrade cyclic nucleotides. Cell homeostasis is maintained by the rapid degradation of cyclic nucleotides after stimulus-induced increases. The enzymes that degrade cyclic nucleotides are called 3′,5′-cyclic nucleotide-specific phosphodiesterases (PDEs).

Eleven PDE gene families (PDE1-PDE11) have been identified so far, based on their distinct amino acid sequences, catalytic and regulatory characteristics, and sensitivity to small molecule inhibitors. These families are coded for by 21 genes; and further multiple splice variants are transcribed from many of these genes. Expression patterns of each of the gene families are distinct. PDEs differ with respect to their affinity for cAMP and cGMP. Activities of different PDEs are regulated by different signals. For example, PDE 1 is stimulated by Ca²⁺/calmodulin. PDE 2 activity is stimulated by cGMP. PDE 3 is inhibited by cGMP. PDE 4 is cAMP specific and is specifically inhibited by rolipram. PDE 5 is cGMP-specific. PDE6 is expressed in retina. Less is known about the expression patterns and functional attributes of the higher number PDEs (7 through 11).

PDE10 sequences were first identified by using bioinformatics and sequence information from other PDE gene families. The PDE10 gene family is distinguished based on its amino acid sequence, functional properties and tissue distribution. The human PDE10 gene is large, over 200 kb, with up to 24 exons coding for each of the splice variants. The amino acid sequence is characterized by two GAF domains (which bind cGMP), a catalytic region, and alternatively spliced N and C termini. Numerous splice variants are possible because of at least 3 alternative exons encoding the N and 2 encoding the C-termini. PDE10A1 is a 779 amino acid protein that hydrolyzes both cAMP and cGMP. The Km values for cAMP and cGMP are 0.05 and 3.0 micromolar, respectively. In addition to human variants, several variants with high homology have been isolated from both rat and mouse tissues and sequence banks.

PDE10 RNA transcripts were initially detected in human testis and brain. Subsequent immunohistochemical analysis revealed that the highest levels of PDE10 are expressed in the basal ganglia. Specifically, striatal neurons in the olfactory tubercle, caudate nucleus and nucleus accumbens are enriched in PDE10. Western blots did not reveal the expression of PDE10 in other brain tissues, although immunoprecipitation of the PDE10 complex was possible in hippocampal and cortical tissues. This suggests that the expression level of PDE10 in these other tissues is 100-fold less than in striatal neurons. Expression in hippocampus is limited to the cell bodies, whereas PDE10 is expressed in terminals, dendrites and axons of striatal neurons.

The tissue distribution of PDE10 indicates that PDE10 inhibitors can be used to raise levels of cAMP and/or cGMP within cells that express the PDE 10 enzyme, for example, neurons that comprise the basal ganglia and therefore would be useful in treating a variety of neuropsychiatric conditions involving the basal ganglia such as obesity, non-insulin dependent diabetes, schizophrenia, bipolar disorder, obsessive compulsive disorder, and the like.

Provided is at least one chemical entity chosen from compounds of Formulas (I) and (II):

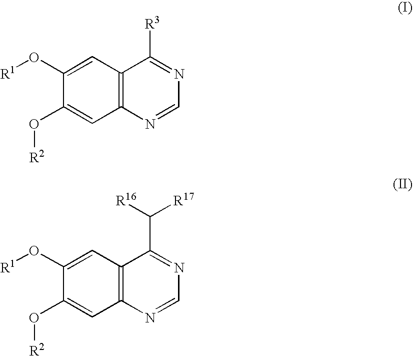

and individual stereoisomers, mixtures of stereoisomers, pharmaceutically acceptable solvates, and pharmaceutically acceptable salts thereof, wherein:
R¹ is chosen from H, alkyl having 1 to 4 carbon atoms, and alkyl having 1 to 4 carbon atoms substituted by at least one halogen;
R² is chosen from H, alkyl having 1 to 4 carbon atoms, and alkyl having 1 to 4 carbon atoms substituted by at least one halogen;
R³ is chosen from: 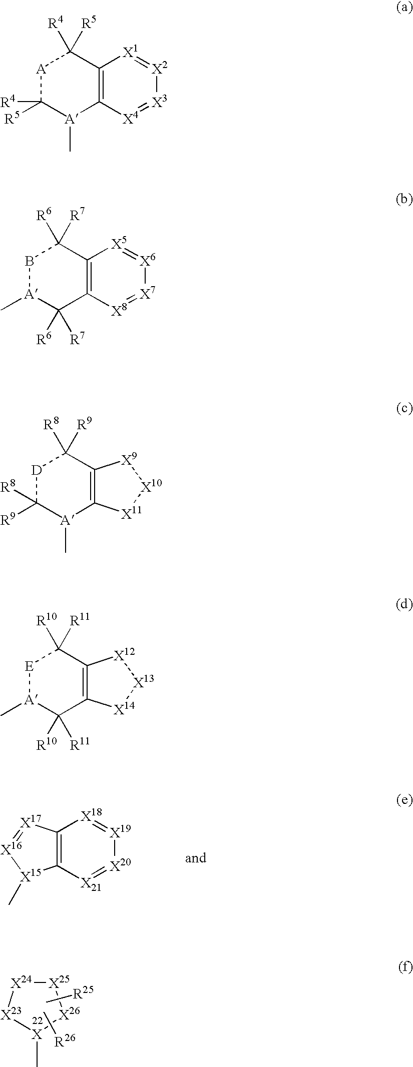
A′ is chosen from N and CH;
----A---- is chosen from a double bond, —CR⁴R⁵—, ═CR⁴—, —CR⁴═, —CR⁴R⁵—CR⁴R⁵—, ═CR⁴—CR⁴R⁵—, —CR⁴R⁵—CR⁴═, —CR⁴═CR⁵—, ═CR⁴—CR⁴═, —CR⁴R⁵—CR⁴R⁵—CR⁴R⁵—, ═CR⁴—CR⁴R⁵—CR⁴R⁵C—, —CR⁴═CR⁴—CR⁴R⁵—, —CR⁴R⁵—CR⁴═CR⁴—, —CR⁴R⁵—CR⁴R⁵—CR⁴═, ═CR⁴—CR⁴═CR⁴—, —CR⁴═CR⁴—CR⁴═, and ═CR⁴—CR⁴R⁵—CR⁴═;
----B---- is chosen from a single bond, —CR⁶R⁷—, —CR⁶═, —CR⁶R⁷—CR⁶R⁷—, —CR⁶R⁷—CR⁶═, —CR⁶═CR⁷—, —CR⁶R⁷—CR⁶R⁷—CR⁶R⁷—, —CR⁶═CR⁶—CR⁶R⁷—, —CR⁶R⁷—CR₆═CR⁶—, —CR⁶R⁷—CR⁶R⁷—CR₆═, and —CR⁶═CR⁶—CR₆═;
----D---- is chosen from —CR⁸R⁹—, ═CR⁸—, —CR⁸═, —CR⁸R⁹—CR⁸R⁹—, ═CR⁸—CR⁸R⁹—, —CR⁸R⁹—CR⁸═, —CR⁸═CR⁹—, ═CR⁸—CR⁸═, —CR⁸R⁹—CR⁸R⁹—CR⁸R⁹—, ═CR⁸—CR⁸R⁹—CR⁸R⁹—, —CR⁸═CR⁸—CR⁸R⁹—, —CR⁸R⁹—CR⁸═CR⁸—, —CR⁸R⁹—CR⁸R⁹—CR₈═, ═CR⁸—CR⁸═CR⁸—, —CR⁸═CR⁸—CR⁸═, and ═CR⁸—CR⁸R⁹—CR⁸═;
----E---- is chosen from —CR¹⁰R¹¹—, —CR¹⁰═, —CR¹⁰R¹¹—CR¹⁰R¹¹—, —CR¹¹—CR¹⁰═, —CR¹⁰═CR¹¹—, —CR¹⁰R¹¹—CR¹⁰R¹¹—CR¹⁰R¹¹—, —CR¹⁰═CR¹⁰—CR¹⁰R¹¹—, —CR¹⁰R¹¹—CR¹⁰═CR¹⁰—, —CR¹⁰R¹¹—CR¹⁰R¹¹—CR¹⁰═, and —CR¹⁰═CR¹⁰—CR¹⁰═;
the dotted lines in the 5-membered ring of formula (c) independently represent a single bond or a double bond; with the proviso there is at least one double bond between X⁹ and X¹⁰ or X¹⁰ and X¹¹;
the dotted lines in the 5-membered ring of formula (d) independently represent a single bond or a double bond; with the proviso there is at least one double bond between X¹² and X¹³ or X¹³ and X¹⁴;
the dotted lines in formula (f) independently represent a single bond or a double bond, with the proviso that when two double bonds are present, they are not adjacent to each other;
X¹, X², X³, X⁴, X⁵, X⁶, X⁷, X⁸, X¹⁸, X¹⁹, X²⁰ and X²¹ are each independently chosen from N and CR¹², and wherein two adjacent X¹—X⁴, X⁵—X⁸, and X¹⁸—X²¹ groups can each be CR¹² in which the two R¹² groups taken together form a fused ring structure chosen from methylenedioxy, ethylenedioxy group, difluoromethylenedioxy, and tetrafluoroethylenedioxy;
X⁹, X¹⁰, X¹¹, X¹², X¹³, and X¹⁴ are each independently chosen from S, O, N, NR¹², C(R¹²)₂, and CR¹²;
X¹⁵, X¹⁶ and X¹⁷ are each independently chosen from N and CR¹² wherein at least two of X¹⁵, X¹⁶ and X¹⁷ are not CR¹²;
X²² is chosen from N, C and CR¹² and X²³, X²⁴, X²⁵, and X²⁶ are each independently chosen from O, S, N, NR¹², C, CHR¹², C(R¹²)₂, and CR¹²; wherein at least two of X²², X²³, X²⁴, X²⁵, and X²⁶ are not chosen from C, CHR¹² and CR¹²;
R⁴, R⁵, R⁶, R⁷, R⁸, R⁹, R¹⁰, and R¹¹ are independently chosen from
   absent,
   H,
   carboxy,
   alkyl having 1 to 8 carbon atoms,
   substituted alkyl having 1 to 8 carbon atoms and substituted with at least one group chosen from halogen, C₁₋₄-alkyl, C₁₋₄-alkoxy, and oxo,
   cycloalkyl having 3 to 12 carbon atoms,
   substituted cycloalkyl having 3 to 12 carbon atoms and substituted with at least one group chosen from halogen, C₁₋₄-alkyl, C₁₋₄-alkoxy, and oxo,
   cycloalkylalkyl having 4 to 12 carbon atoms, and
   substituted cycloalkylalkyl having 4 to 12 carbon atoms and substituted with at least one group chosen from halogen, C₁₋₄-alkyl, C₁₋₄-alkoxy, and oxo, or
R⁴ and R⁵ together form a cycloalkyl group chosen from 3 to 8 membered spiro cycloalkyl and 3 to 8 membered fused cycloalkyl, or
R⁶ and R⁷ together form a cycloalkyl group chosen from 3 to 8 membered spiro cycloalkyl and 3 to 8 membered fused cycloalkyl, or
R⁸ and R⁹ together form a cycloalkyl group chosen from 3 to 8 membered spiro cycloalkyl and 3 to 8 membered fused cycloalkyl, or
R¹⁰ and R¹¹ together form a cycloalkyl group chosen from 3 to 8 membered spiro cycloalkyl and 3 to 8 membered fused cycloalkyl, or
one or more of R⁴ and R⁵ and the carbon atom to which they are attached form a C(═O) group, or
one or more of R⁶ and R⁷ and the carbon atom to which they are attached form a C(═O) group, or
one or more of R⁸ and R⁹ and the carbon atom to which they are attached form a C(═O) group, or
one or more of R¹⁰ and R¹¹ and the carbon atom to which they are attached, in each case form a C(═O) group,
R¹² is chosen from
   H,
   alkyl having up to 12 carbon atoms,
   substituted alkyl having up to 12 carbon atoms and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, or —NHCSNHR¹³ and wherein optionally one or more —CH₂CH₂— groups is replaced in each case by a group chosen from —CH═CH— and —C≡C—;
   cycloalkyl having 3 to 12 carbon atoms,
   substituted cycloalkyl having 3 to 12 carbon atoms and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from —CH═CH— and —C≡C—;
   cycloalkylalkyl having up to 12 carbon atoms,
   substituted cycloalkylalkyl having up to 12 carbon atoms and substituted and
   substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from —CH═CH— and —C≡C—;
   heterocyclyl,
   heterocyclyl substituted with at least one group chosen from halogen, C₆₋₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   heteroaryl,
   heteroaryl substituted with at least one group chosen from halogen, C₆₋₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   aryl having 6 to 14 carbon atoms,
   substituted aryl having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
   arylalkyl having 7 to 16 carbon atoms,
   substituted arylalkyl having 7 to 16 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
   heteroarylalkyl wherein the heteroaryl portion has 5 to 10 ring atoms in which at least 1 ring atom is a heteroatom and the alkyl portion has 1 to 3 carbon atoms,
   substituted heteroarylalkyl wherein the heteroaryl portion has 5 to 10 ring atoms in which at least 1 ring atom is a heteroatom and the alkyl portion has 1 to 3 carbon atoms and wherein the heteroaryl portion is substituted by at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   aryloxy having 6 to 14 carbon atoms,
   substituted aryloxy having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy;
   heteroaryloxy having 5 to 10 ring atoms in which at least 1 ring atom is a heteroatom,
   substituted heteroaryloxy having 5 to 10 ring atoms in which at least 1 ring atom is a heteroatom, and substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   halogen, hydroxy, C₁₋₄-alkoxy, C₁₋₄-alkoxy-C₁₋₄-alkoxy, C₄₋₁₂-cycloalkylalkyloxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, —C₁₋₄-alkylthio, C₁₋₄-alkylsulphinyl, C₁₋₄-alkylsulphonyl, —SO₂NHR¹⁹, —SO₂NR¹⁸R¹⁹, —SO₂R²⁰, —NHSO₂R¹³, —NR¹³COR¹³, —CONHR¹³, —CONR¹³R¹⁹, —NHCONHR¹³, —OCONHR¹³, —NHCOOR¹³, —SCONHR¹³, —SCSNHR¹³, and —NHCSNHR¹³;
R¹³ is chosen from
   H,
   alkyl having 1 to 8 carbon atoms,
   substituted alkyl having 1 to 8 carbon atoms and substituted with at least one group chosen from halogen, C₁₋₄-alkyl, C₁₋₄-alkoxy, and oxo,
   cycloalkyl having 3 to 12 carbon atoms,
   substituted cycloalkyl having 3 to 12 carbon atoms and substituted with at least one group chosen from halogen, C₁₋₄-alkyl, C₁₋₄-alkoxy, and oxo;
   cycloalkylalkyl having 4 to 12 carbon atoms, and
   substituted cycloalkylalkyl having 4 to 12 carbon atoms and substituted with at least one group chosen from halogen, C₁₋₄-alkyl, C₁₋₄-alkoxy, and oxo;
R¹⁶ is chosen from
   aryl having 6 to 14 carbon atoms,
   substituted aryl having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
   heteroaryl having 5 to 10 ring atoms in which at least 1 ring atom is a heteroatom,
   substituted heteroaryl having 5 to 10 ring atoms in which at least 1 ring atom is a heteroatom and substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   heterocyclyl,
   substituted heterocyclyl substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   carbocyclic, and
   substituted carbocyclic substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy;
R¹⁷ is chosen from
   alkyl having 1 to 12 carbon atoms,
   substituted alkyl having 1 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸ wherein optionally one or more —CH₂— groups is, in each case independently, replaced by —O—, —S—, or —NH— and wherein optionally one or more —CH₂CH₂— groups is replaced in each case by —CH═CH— or —C≡C—,
   cycloalkyl having 3 to 12 carbon atoms,
   substituted cycloalkyl having 3 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸, wherein optionally one or more —CH₂— groups is, in each case independently, replaced by —O—, —S—, or —NH— and wherein optionally one or more —CH₂CH₂— groups is replaced in each case by —CH═CH— or —C≡C—,
   cycloalkylalkyl having 4 to 12 carbon atoms,
   substituted cycloalkylalkyl having 4 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸ wherein optionally one or more —CH₂— groups is, in each case independently, replaced by —O—, —S—, or —NH— and wherein optionally one or more —CH₂CH₂— groups is replaced in each case by —CH═CH— or —C≡C—,
   halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and NHCSNHR¹⁸;
R¹⁸ is chosen from
   H,
   alkyl having 1 to 8 carbon atoms, and
   substituted alkyl having 1 to 8 carbon atoms substituted with at least one group chosen from halogen, C₁₋₄-alkyl, C₁₋₄-alkoxy, and oxo;
R¹⁹ is chosen from
   H,
   alkyl having 1 to 8 carbon atoms,
   substituted alkyl having 1 to 8 carbon atoms and substituted with at least one group chosen from halogen, C₁₋₄-alkyl, C₁₋₄-alkoxy, and oxo,
   cycloalkyl having 3 to 10 carbon atoms,
   substituted cycloalkyl having 3 to 10 carbon atoms and substituted with at least one group chosen from halogen, C₁₋₄-alkyl, C₁₋₄-alkoxy, and oxo,
   cycloalkylalkyl having 4 to 12 carbon atoms,
   substituted cycloalkylalkyl having 4 to 12 carbon atoms and substituted with at least one group chosen from halogen, C₁₋₄-alkyl, C₁₋₄-alkoxy, and oxo,
   heteroaryl,
   heteroaryl substituted with at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
   heterocyclyl, and
   heterocyclyl substituted with at least one group chosen from halogen, C₆₋₁₄
   aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
R²⁰ is chosen from
   heterocyclyl, and
   heterocyclyl substituted by at least one group chosen from halogen, C₆₋₁₄-aryl-C₁₋₄-alkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
R²⁵ and R²⁶ are independently chosen from
   H,
   carboxy,
   alkyl having 1 to 8 carbon atoms,
   substituted alkyl having 1 to 8 carbon atoms and substituted with at least one group chosen from halogen, C₁₋₄-alkyl, C₁₋₄-alkoxy, and oxo,
   cycloalkyl having 3 to 12 carbon atoms,
   substituted cycloalkyl having 3 to 12 carbon atoms and substituted with at least one group chosen from halogen, C₁₋₄-alkyl, C₁₋₄-alkoxy, and oxo,
   cycloalkylalkyl having 4 to 12 carbon atoms,
   cycloalkylalkyl having 4 to 12 carbon atoms substituted with at least one group chosen from halogen, C₁₋₄-alkyl, C₁₋₄-alkoxy, and oxo, or
R²⁵ and R²⁶ together form a cycloalkyl group, spiro or fused, having 3 to 8 carbon atoms, or
R²⁵ and R²⁶ and the carbon atom to which they are attached form a C(═O) group;
   with the proviso that
   said compound of Formulas (I) and (II) is not chosen from
6,7-dimethoxy-4-(2-methyl-3,4-dihydroquinolin-1- (2H)-yl)quinazoline;
4-(7-bromo-3,4-dihydroquinolin-1- (2H)-yl)-6,7-dimethoxyquinazoline;
4-(5-bromo-3,4-dihydroquinolin-1(2H)-yl)-6,7-dimethoxyquinazoline;
6,7-dimethoxy-4-[7-(trifluoromethyl)-3,4-dihydroquinolin-1- (2H)-yl]quinazoline;
6,7-dimethoxy-4-(6-methyl-3,4-dihydroquinolin-1(2H)-yl)quinazoline;
4-(3,4-dihydroquinolin-1(2H)-yl)-6,7-dimethoxyquinazoline;
8-bromo-1-(6,7-dimethoxyquinazolin-4-yl)-2,3,4,5-tetrahydro-1H-1-benzazepine;
9-bromo-1-(6,7-dimethoxyquinazolin-4-yl)-2,3,4,5-tetrahydro-1H-1-benzazepine;
1-(6,7-dimethoxyquinazolin-4-yl)-2,3,4,5-tetrahydro-1H-1-benzazepine;
1-(6,7-dimethoxyquinazolin-4-yl)-1H-indole-3-carbaldehyde; 4-(1H-indol-1-yl)-6,7-dimethoxyquinazoline;
4-(1-H-benzotriazol-1-yl)-6,7-dimethoxyquinazoline;
4-(1-H-benzimidazol-1-yl)-6,7-dimethoxyquinazoline;
4-(1-H-indazol-1-yl)-6,7-dimethoxyquinazoline;
4-(5-fluorophenyl)-2-[4-(methylsulfonyl)phenyl)-1H-imidazol-4-yl)-6,7-dimethoxyquinazoline;
4-(1-cyclopropylmethyl)-4-(4-fluorophenyl)-1H-imidazol-5-yl)-6,7-dimethoxyquinazoline;
4-(5-(4-fluorophenyl)-3-phenyl-1H-1,2,4-triazol-1-yl)-6,7-dimethoxyquinazoline;
1-(6,7-dimethoxy-4quinazolinyl)-1H-pyrazole-3-amine;
N-[2-(6,7-dimethoxy-quinazolin-4-yl)-1,2,3,4-tetrahydro-isoquinolin-7-yl]-2,2-dimethyl-propionamide;
N-[2-(6,7-dimethoxy-quinazoline-4-yl)-1,2,3,4-tetrahydro-isoquinolin-7-yl]-acetamide;
6,7-dimethoxy-4-[8-(morpholine-4-sulfonyl)-3,4-dihydro-1H-isoquinolin-2-yl]-quinazoline;
6,7-dimethoxy-4-[8-(4-methyl-piperazine-1-sulfonyl)-3,4-dihydro-1H-isoquinolin-2-yl]-quinazoline;
4-(7,8-dimethoxy-3,4-dihydro-1H-isoquinolin-2-yl)-6-ethoxy-7-methoxy-quinazoline;
4-(6,7-dimethoxy-3,4-dihydro-1H-isoquinolin-2-yl)-6-ethoxy-7-methoxy-quinazoline;
4-(6,7-dimethoxy-3-methyl-3,4-dihydro-1H-isoquinolin-2-yl)-6-ethoxy-7-methoxy-quinazoline;
4-(3,4-dihydro-1H-isoquinolin-2-yl)-6-ethoxy-7-methoxy-quinazoline;
2-(6,7-dimethoxy-quinazolin-4-yl)-1,2,3,4-tetrahydro-isoquinolin-7-yl-amine; and
6,7-dimethoxy-4-(3-propyl-3,4-dihydro-1H-isoquinolin-2-yl)-quinazoline.

Also provided is a pharmaceutical composition comprising at least one chemical entity described herein and a pharmaceutically acceptable carrier, provided that the at least one chemical entity is not chosen from 6,7-dimethoxy-4-(2-methyl-3,4-dihydroquinolin-1- (2H)-yl)quinazoline;
4-(7-bromo-3,4-dihydroquinolin-1(2H)-yl)-6,7-dimethoxyquinazoline;
4-(5-bromo-3,4-dihydroquinolin-1(2H)-yl)-6,7-dimethoxyquinazoline;
6,7-dimethoxy-4-[7-(trifluoromethyl)-3,4-dihydroquinolin-1(2H)-yl]quinazoline;
6,7-dimethoxy-4-(6-methyl-3,4-dihydroquinolin-1(2H)-yl)quinazoline;
4-(3,4-dihydroquinolin-1(2H)-yl)-6,7-dimethoxyquinazoline;
8-bromo-1-(6,7-dimethoxyquinazolin-4-yl)-2,3,4,5-tetrahydro-1H-1-benzazepine;
9-bromo-1-(6,7-dimethoxyquinazolin-4-yl)-2,3,4,5-tetrahydro-1H-1-benzazepine;
1-(6,7-dimethoxyquinazolin-4-yl)-2,3,4,5-tetrahydro-1H-1-benzazepine;
1-(6,7-dimethoxyquinazolin-4-yl)-1H-indole-3-carbaldehyde; 4-(1H-indol-1-yl)-6,7-dimethoxyquinazoline;
4-(1-H-benzotriazol-1-yl)-6,7-dimethoxyquinazoline;
4-(1-H-benzimidazol-1-yl)-6,7-dimethoxyquinazoline;
4-(1-H-indazol-1-yl)-6,7-dimethoxyquinazoline;
4-(5-fluorophenyl)-2-[4-(methylsulfonyl)phenyl)-1H-imidazol-4-yl)-6,7-dimethoxyquinazoline;
4-(1-cyclopropylmethyl)-4-(4-fluorophenyl)-1H-imidazol-5-yl)-6,7-dimethoxyquinazoline;
4-(5-(4-fluorophenyl)-3-phenyl-1H-1,2,4-triazol-1-yl)-6,7-dimethoxyquinazoline;
1-(6,7-dimethoxy-4quinazolinyl)-1H-pyrazole-3-amine;
N-[2-(6,7-dimethoxy-quinazolin-4-yl)-1,2,3,4-tetrahydro-isoquinolin-7-yl]-2,2-dimethyl-propionamide;
N-[2-(6,7-dimethoxy-quinazoline-4-yl)-1,2,3,4-tetrahydro-isoquinolin-7-yl]-acetamide;
6,7-dimethoxy-4-[8-(morpholine-4-sulfonyl)-3,4-dihydro-1H-isoquinolin-2-yl]-quinazoline;
6,7-dimethoxy-4-[8-(4-methyl-piperazine-1-sulfonyl)-3,4-dihydro-1H-isoquinolin-2-yl]-quinazoline;
4-(7,8-dimethoxy-3,4-dihydro-1H-isoquinolin-2-yl)-6-ethoxy-7-methoxy-quinazoline;
4-(6,7-dimethoxy-3,4-dihydro-1H-isoquinolin-2-yl)-6-ethoxy-7-methoxy-quinazoline;
4-(6,7-dimethoxy-3-methyl-3,4-dihydro-1H-isoquinolin-2-yl)-6-ethoxy-7-methoxy-quinazoline;
4-(3,4-dihydro-1H-isoquinolin-2-yl)-6-ethoxy-7-methoxy-quinazoline;
2-(6,7-dimethoxy-quinazolin-4-yl)-1,2,3,4-tetrahydro-isoquinolin-7-yl-amine; and
6,7-dimethoxy-4-(3-propyl-3,4-dihydro-1H-isoquinolin-2-yl)-quinazoline, and

individual stereoisomers, mixtures of stereoisomers, pharmaceutically acceptable solvates, and pharmaceutically acceptable salts thereof

In certain embodiments, the pharmaceutical composition comprises one chemical entity described herein and a pharmaceutically acceptable carrier, provided that the at least one chemical entity is not chosen from 6,7-dimethoxy-4-(2-methyl-3,4-dihydroquinolin-1- (2H)-yl)quinazoline;
4-(7-bromo-3,4-dihydroquinolin-1(2H)-yl)-6,7-dimethoxyquinazoline;
4-(5-bromo-3,4-dihydroquinolin-1- (2H)-yl)-6,7-dimethoxyquinazoline;
6,7-dimethoxy-4-[7-(trifluoromethyl)-3,4-dihydroquinolin-1(2H)-yl]quinazoline;
6,7-dimethoxy-4-(6-methyl-3,4-dihydroquinolin-1(2H)-yl)quinazoline;
4-(3,4-dihydroquinolin-1- (2H)-yl)-6,7-dimethoxyquinazoline;
8-bromo-1-(6,7-dimethoxyquinazolin-4-yl)-2,3,4,5-tetrahydro-1H-1-benzazepine;
9-bromo-1-(6,7-dimethoxyquinazolin-4-yl)-2,3,4,5-tetrahydro-1H-1-benzazepine;
1-(6,7-dimethoxyquinazolin-4-yl)-2,3,4,5-tetrahydro-1H-1-benzazepine;
1-(6,7-dimethoxyquinazolin-4-yl)-1H-indole-3-carbaldehyde; 4-(1H-indol-1-yl)-6,7-dimethoxyquinazoline;
4-(1-H-benzotriazol-1-yl)-6,7-dimethoxyquinazoline;
4-(1-H-benzimidazol-1-yl)-6,7-dimethoxyquinazoline;
4-(1-H-indazol-1-yl)-6,7-dimethoxyquinazoline;
4-(5-fluorophenyl)-2-[4-(methylsulfonyl)phenyl)-1H-imidazol-4-yl)-6,7-dimethoxyquinazoline;
4-(1-cyclopropylmethyl)-4-(4-fluorophenyl)-1H-imidazol-5-yl)-6,7-dimethoxyquinazoline;
4-(5-(4-fluorophenyl)-3-phenyl-1H-1,2,4-triazol-1-yl)-6,7-dimethoxyquinazoline;
1-(6,7-dimethoxy-4quinazolinyl)-1H-pyrazole-3-amine;
N-[2-(6,7-dimethoxy-quinazolin-4-yl)-1,2,3,4-tetrahydro-isoquinolin-7-yl]-2,2-dimethyl-propionamide;
N-[2-(6,7-dimethoxy-quinazoline-4-yl)-1,2,3,4-tetrahydro-isoquinolin-7-yl]-acetamide;
6,7-dimethoxy-4-[8-(morpholine-4-sulfonyl)-3,4-dihydro-1H-isoquinolin-2-yl]-quinazoline;
6,7-dimethoxy-4-[8-(4-methyl-piperazine-1-sulfonyl)-3,4-dihydro-1H-isoquinolin-2-yl]-quinazoline;
4-(7,8-dimethoxy-3,4-dihydro-1H-isoquinolin-2-yl)-6-ethoxy-7-methoxy-quinazoline;
4-(6,7-dimethoxy-3,4-dihydro-1H-isoquinolin-2-yl)-6-ethoxy-7-methoxy-quinazoline;
4-(6,7-dimethoxy-3-methyl-3,4-dihydro-1H-isoquinolin-2-yl)-6-ethoxy-7-methoxy-quinazoline;
4-(3,4-dihydro-1H-isoquinolin-2-yl)-6-ethoxy-7-methoxy-quinazoline;
2-(6,7-dimethoxy-quinazolin-4-yl)-1,2,3,4-tetrahydro-isoquinolin-7-yl-amine; and
6,7-dimethoxy-4-(3-propyl-3,4-dihydro-1H-isoquinolin-2-yl)-quinazoline, and

individual stereoisomers, mixtures of stereoisomers, pharmaceutically acceptable solvates, and pharmaceutically acceptable salts thereof.

Also provided is a method of inhibiting PDE10 enzyme in a patient in need thereof comprising administering to said patient an effective amount of at least one chemical entity chosen from compounds of Formulas (I) and (II):

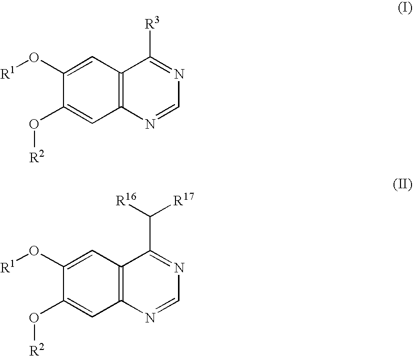

and individual stereoisomers, mixtures of stereoisomers, pharmaceutically acceptable solvates, and pharmaceutically acceptable salts thereof, wherein:
R¹ is chosen from H, alkyl having 1 to 4 carbon atoms, and alkyl having 1 to 4 carbon atoms substituted by at least one halogen;
R² is chosen from H, alkyl having 1 to 4 carbon atoms, and alkyl having 1 to 4 carbon atoms substituted by at least one halogen;
R³ is chosen from: 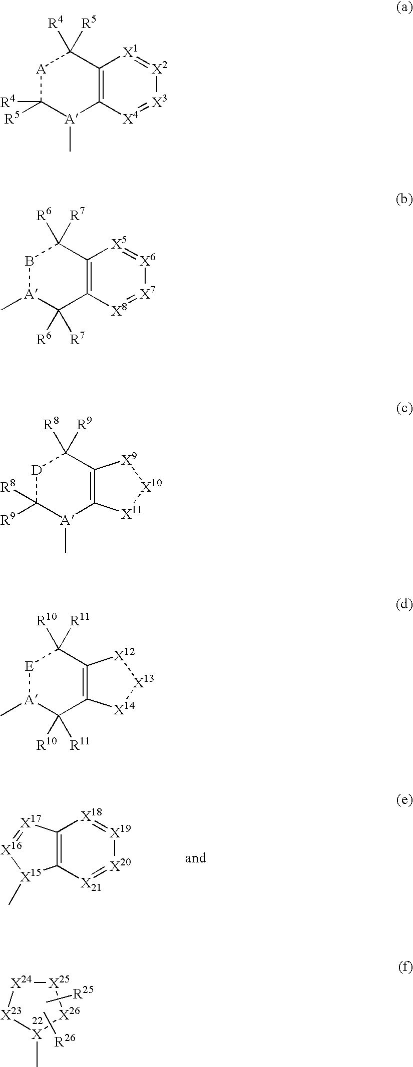
A′ is chosen from N and CH;
----A---- is chosen from a double bond, —CR⁴R⁵—, ═CR⁴—, —CR⁴═, —CR⁴R⁵—CR⁴R⁵—, ═CR⁴—CR⁴R⁵—, —CR⁴R⁵—CR⁴═, —CR⁴═CR⁵—, ═CR⁴—CR⁴═, —CR⁴R⁵—CR⁴R⁵—CR⁴R⁵—, ═CR⁴—CR⁴R⁵—CR⁴R⁵—, —CR⁴═CR⁴—CR⁴R⁵—, —CR⁴R⁵—CR⁴═CR⁴—, —CR⁴R⁵—CR⁴R⁵—CR⁴═, ═CR⁴—CR⁴═CR⁴—, —CR⁴═CR⁴—CR⁴═, and ═CR⁴—CR⁴R⁵—CR⁴═;
----B---- is chosen from a single bond, —CR⁶R⁷—, —CR⁶═, —CR⁶R⁷—CR⁶R⁷—, —CR⁶R⁷—CR⁶═, —CR⁶═CR⁷—, —CR⁶R⁷—CR⁶R⁷—CR⁶R⁷—, —CR⁶═CR⁶—CR⁶R⁷—, —CR⁶R⁷—CR⁶═CR⁶—, —CR⁶R⁷—CR⁶R⁷CR⁶═, and —CR⁶═CR⁶—CR⁶═;
----D---- is chosen from —CR⁸R⁹—, ═CR⁸—, —CR⁸═, —CR⁸R⁹—CR⁸R⁹—, ═CR⁸—CR⁸R⁹—, —CR⁸R⁹—CR⁸═, —CR⁸═CR⁹—, ═CR⁸—CR⁸═, —CR⁸R⁹—CR⁸R⁹—CR⁸R⁹—, ═CR⁸—CR⁸R⁹—CR⁸R⁹—, —CR⁸═CR⁸—CR⁸R⁹—, —CR⁸R⁹—CR⁸═CR⁸—, —CR⁸R⁹—CR⁸R⁹—CR⁸═, ═CR⁸—CR⁸═CR⁸—, —CR⁸═CR⁸—CR⁸═, and ═CR⁸—CR⁸R⁹—CR⁸═;
----E---- is chosen from —CR¹⁰R¹¹—, —CR¹⁰═, —CR¹⁰R¹¹—CR¹⁰R¹¹—, —CR¹⁰R¹¹—CR¹⁰═, —CR¹⁰═CR¹¹—, —CR¹⁰R¹¹—CR¹⁰R¹¹—CR¹⁰R¹¹—, —CR¹⁰═CR¹⁰—CR¹⁰R¹¹—, —CR¹⁰R¹¹—CR¹⁰═CR¹⁰—, —CR¹⁰R¹¹—CR¹⁰R¹¹—CR¹⁰═, and —CR¹⁰═CR¹⁰—CR¹⁰═;
the dotted lines in the 5-membered ring of formula (c) independently represent a single bond or a double bond; with the proviso there is at least one double bond between X⁹ and X¹⁰ or X¹⁰ and X¹¹;
the dotted lines in the 5-membered ring of formula (d) independently represent a single bond or a double bond; with the proviso there is at least one double bond between X¹² and X¹³ or X¹³ and X¹⁴;
the dotted lines in formula (f) independently represent a single bond or a double bond, with the proviso that when two double bonds are present, they are not adjacent to each other;
X¹, X², X³, X⁴, X⁵, X⁶, X⁷, X⁸, X¹⁸, X¹⁹, X²⁰ and X²¹ are each independently chosen from N and CR¹², and wherein two adjacent X¹—X⁴, X⁵—X⁸, and X¹⁸—X²¹ groups can each be CR¹² in which the two R¹² groups taken together form a fused ring structure chosen from methylenedioxy, ethylenedioxy group, difluoromethylenedioxy, and tetrafluoroethylenedioxy;
X⁹, X¹⁰, X¹¹, X¹², X¹³, and X¹⁴ are each independently chosen from S, O, N, NR¹², C(R¹²)₂, and CR¹²;
X¹⁵, X¹⁶ and X¹⁷ are each independently chosen from N and CR¹² wherein at least two of X¹⁵, X¹⁶ and X¹⁷ are not CR¹²;
X²² is chosen from N, C and CR¹² and X²³, X²⁴, X²⁵, and X²⁶ are each independently chosen from O, S, N, NR¹², C, CHR¹², C(R¹²)₂, and CR¹²; wherein at least two of X²², X²³, X²⁴, X²⁵, and X²⁶ are not chosen from C, CHR¹² and CR¹²;
R⁴, R⁵, R⁶, R⁷, R⁸, R⁹, R¹⁰, and R¹¹ are independently chosen from
   absent,
   H,
   carboxy,
   alkyl having 1 to 8,
   substituted alkyl having 1 to 8 carbon atoms and substituted with at least one group chosen from halogen, C₁₋₄-alkyl, C₁₋₄-alkoxy, and oxo,
   cycloalkyl having 3 to 12 carbon atoms,
   substituted cycloalkyl having 3 to 12 carbon atoms and substituted with at least one group chosen from halogen, C₁₋₄-alkyl, C₁₋₄-alkoxy, and oxo,
   cycloalkylalkyl having 4 to 12 carbon atoms, and
   substituted cycloalkylalkyl having 4 to 12 carbon atoms and substituted with at least one group chosen from halogen, C₁₋₄-alkyl, C₁₋₄-alkoxy, and oxo, or
R⁴ and R⁵ together form a cycloalkyl group chosen from 3 to 8 membered spiro cycloalkyl and 3 to 8 membered fused cycloalkyl, or
R⁶ and R⁷ together form a cycloalkyl group chosen from 3 to 8 membered spiro cycloalkyl and 3 to 8 membered fused cycloalkyl, or
R⁸ and R⁹ together form a cycloalkyl group chosen from 3 to 8 membered spiro cycloalkyl and 3 to 8 membered fused cycloalkyl, or
R¹⁰ and R¹¹ together form a cycloalkyl group chosen from 3 to 8 membered spiro cycloalkyl and 3 to 8 membered fused cycloalkyl, or
one or more of R⁴ and R⁵ and the carbon atom to which they are attached form a C(═O) group, or
one or more of R⁶ and R⁷ and the carbon atom to which they are attached form a C(═O) group, or
one or more of R⁸ and R⁹ and the carbon atom to which they are attached form a C(═O) group, or
one or more of R¹⁰ and R¹¹ and the carbon atom to which they are attached, in each case form a C(═O) group,
R¹² is chosen from
   H,
   alkyl having up to 12 carbon atoms,
   substituted alkyl having up to 12 carbon atoms and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR³, —OCONHR¹³, —NHCOOR¹³, —SCONHR¹³, —SCSNHR¹³, or —NHCSNHR¹³ and wherein optionally one or more —CH₂CH₂— groups is replaced in each case by a group chosen from —CH═CH— and —C≡C—;
   cycloalkyl having 3 to 12 carbon atoms,
   substituted cycloalkyl having 3 to 12 carbon atoms and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from —CH═CH— and —C≡C—;
   cycloalkylalkyl having up to 12 carbon atoms,
   substituted cycloalkylalkyl having up to 12 carbon atoms and substituted and
   substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from —CH═CH— and —C≡C—;
   heterocyclyl,
   heterocyclyl substituted with at least one group chosen from halogen, C₆₋₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   heteroaryl,
   heteroaryl substituted with at least one group chosen from halogen, C₆₋₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   aryl having 6 to 14 carbon atoms,
   substituted aryl having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
   arylalkyl having 7 to 16 carbon atoms,
   substituted arylalkyl having 7 to 16 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
   heteroarylalkyl wherein the heteroaryl portion has 5 to 10 ring atoms in which at least 1 ring atom is a heteroatom and the alkyl portion has 1 to 3 carbon atoms,
   substituted heteroarylalkyl wherein the heteroaryl portion has 5 to 10 ring atoms in which at least 1 ring atom is a heteroatom and the alkyl portion has 1 to 3 carbon atoms and wherein the heteroaryl portion is
   substituted by at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   aryloxy having 6 to 14 carbon atoms,
   substituted aryloxy having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy;
   heteroaryloxy having 5 to 10 ring atoms in which at least 1 ring atom is a heteroatom,
   substituted heteroaryloxy having 5 to 10 ring atoms in which at least 1 ring atom is a heteroatom, and substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   halogen, hydroxy, C₁₋₄-alkoxy, C₁₋₄-alkoxy-C₁₋₄-alkoxy, C₄₋₁₂-cycloalkylalkyloxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, —C₁₋₄-alkylthio, C₁₋₄-alkylsulphinyl, C₁₋₄-alkylsulphonyl, —SO₂NHR¹⁹, —SO₂NR¹⁸R¹⁹, —SO₂R²⁰, —NHSO₂R¹³, —NR¹³COR¹³, —CONHR¹³, —CONR¹³R¹⁹, —NHCONHR¹³, —OCONHR¹³, —NHCOOR¹³, —SCONHR¹³, —SCSNHR¹³, and —NHCSNHR¹³;
R¹³ is chosen from
   H,
   alkyl having 1 to 8 carbon atoms,
   substituted alkyl having 1 to 8 carbon atoms and substituted with at least one group chosen from halogen, C₁₋₄-alkyl, C₁₋₄-alkoxy, and oxo,
   cycloalkyl having 3 to 12 carbon atoms,
   substituted cycloalkyl having 3 to 12 carbon atoms and substituted with at least one group chosen from halogen, C₁₋₄-alkyl, C₁₋₄-alkoxy, and oxo;
   cycloalkylalkyl having 4 to 12 carbon atoms, and
   substituted cycloalkylalkyl having 4 to 12 carbon atoms and substituted with at least one group chosen from halogen, C₁₋₄-alkyl, C₁₋₄-alkoxy, and oxo;
R¹⁶ is chosen from
   aryl having 6 to 14 carbon atoms,
   substituted aryl having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
   heteroaryl having 5 to 10 ring atoms in which at least 1 ring atom is a heteroatom,
   substituted heteroaryl having 5 to 10 ring atoms in which at least 1 ring atom is a heteroatom and substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   heterocyclyl,
   substituted heterocyclyl substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   carbocyclic, and
   substituted carbocyclic substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy;
R¹⁷ is chosen from
   alkyl having 1 to 12 carbon atoms,
   substituted alkyl having 1 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸ wherein optionally one or more —CH₂— groups is, in each case independently, replaced by —O—, —S—, or —NH— and wherein optionally one or more —CH₂CH₂— groups is replaced in each case by —CH═CH— or —C≡C—,
   cycloalkyl having 3 to 12 carbon atoms,
   substituted cycloalkyl having 3 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸, wherein optionally one or more —CH₂— groups is, in each case independently, replaced by —O—, —S—, or —NH— and wherein optionally one or more —CH₂CH₂— groups is replaced in each case by —CH═CH— or —C≡C—, cycloalkylalkyl,
   substituted cycloalkylalkyl substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹³, —CONHR¹⁸, —NHCONHR¹⁸, —OCONHR¹⁸, —NHCOOR¹⁸, —SCONHR¹⁸, —SCSNHR¹⁸, and —NHCSNHR¹⁸ wherein optionally one or more —CH₂— groups is, in each case independently, replaced by —O—, —S—, or —NH— and wherein optionally one or more —CH₂CH₂— groups is replaced in each case by —CH═CH— or —C≡C—,
   halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and NHCSNHR¹⁸;
R¹⁸ is chosen from
   H,
   alkyl having 1 to 8 carbon atoms, and
   substituted alkyl having 1 to 8 carbon atoms substituted with at least one group chosen from halogen, C₁₋₄-alkyl, C₁₋₄-alkoxy, and oxo;
R¹⁹ is chosen from
   H,
   alkyl having 1 to 8 carbon atoms,
   substituted alkyl having 1 to 8 carbon atoms and substituted with at least one group chosen from halogen, C₁₋₄-alkyl, C₁₋₄-alkoxy, and oxo,
   cycloalkyl having 3 to 10 carbon atoms,
   substituted cycloalkyl having 3 to 10 carbon atoms and substituted with at least one group chosen from halogen, C₁₋₄-alkyl, C₁₋₄-alkoxy, and oxo,
   cycloalkylalkyl having 4 to 12 carbon atoms,
   substituted cycloalkylalkyl having 4 to 12 carbon atoms and substituted with at least one group chosen from halogen, C₁₋₄-alkyl, C₁₋₄-alkoxy, and oxo,
   heteroaryl,
   heteroaryl substituted with at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
   heterocyclyl, and
   heterocyclyl substituted with at least one group chosen from halogen, C₆₋₁₄
   aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
R²⁰ is chosen from
   heterocyclyl, and
   heterocyclyl substituted by at least one group chosen from halogen, C₆₋₁₄-aryl-C₁₋₄-alkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
R²⁵ and R²⁶ are independently chosen from
   H,
   carboxy,
   alkyl having 1 to 8 carbon atoms,
   substituted alkyl having 1 to 8 carbon atoms and substituted with at least one group chosen from halogen, C₁₋₄-alkyl, C₁₋₄-alkoxy, and oxo,
   cycloalkyl having 3 to 12 carbon atoms,
   substituted cycloalkyl having 3 to 12 carbon atoms and substituted with at least one group chosen from halogen, C₁₋₄-alkyl, C₁₋₄-alkoxy, and oxo,
   cycloalkylalkyl having 4 to 12 carbon atoms,
   cycloalkylalkyl having 4 to 12 carbon atoms substituted with at least one group chosen from halogen, C₁₋₄-alkyl, C₁₋₄-alkoxy, and oxo, or
R²⁵ and R²⁶ together form a cycloalkyl group, spiro or fused, having 3 to 8 carbon atoms, or
R²⁵ and R²⁶ and the carbon atom to which they are attached form a C(═O) group.

Also provided is a method of inhibiting PDE10 enzyme in a patient in need thereof comprising administering to said patient an effective amount of at least one chemical entity chosen from compounds of Formulas (I) and (II):

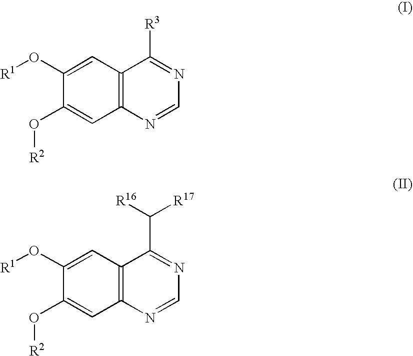

and individual stereoisomers, mixtures of stereoisomers, pharmaceutically acceptable solvates, and pharmaceutically acceptable salts thereof, wherein:
R¹ is chosen from H, alkyl having 1 to 4 carbon atoms, and alkyl having 1 to 4 carbon atoms substituted by at least one halogen;
R² is chosen from H, alkyl having 1 to 4 carbon atoms, and alkyl having 1 to 4 carbon atoms substituted by at least one halogen;
R³ is chosen from: 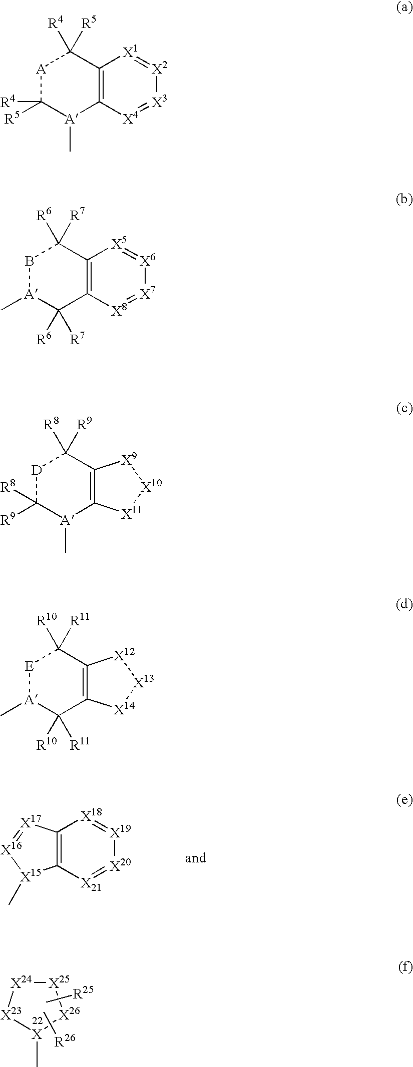
A′ is chosen from N and CH;
----A---- is chosen from a double bond, —CR⁴R⁵—, ═CR⁴—, —CR⁴═, —CR⁴R⁵—CR⁴R⁵—, ═CR⁴—CR⁴R⁵—, —CR⁴R⁵—CR⁴═, ═, —CR⁴═CR⁵—, ═CR⁴—CR⁴═, —CR⁴R⁵—CR⁴R⁵—CR⁴R⁵—, ═CR⁴—CR⁴R⁵—CR⁴R⁵—, —CR⁴═CR⁴—CR⁴R⁵—, —CR⁴R⁵—CR⁴═CR⁴—, —CR⁴R⁵—CR⁴R⁵—CR⁴═, ═CR⁴—CR⁴═, —CR⁴═, —CR⁴═CR⁴—CR⁴═, and ═CR⁴—CR⁴R⁵—CR⁴═;
----B---- is chosen from a single bond, —CR⁶R⁷—, —CR⁶═, —CR⁶R⁷—CR⁶R⁷—, —CR⁶R⁷—CR⁶═, —CR⁶═CR⁷—CR⁶R⁷—CR⁶R⁷—CR⁶R⁷—, —CR⁶—CR⁶R⁷—, —CR⁶R⁷—CR⁶═CR⁶—, —CR⁶R⁷—CR⁶R⁷—CR⁶═, and —CR⁶═CR⁶—CR⁶═;
----D---- is chosen from —CR⁸R⁹—, ═CR⁸—, —CR⁸═, —CR⁸R⁹—CR⁸R⁹—, ═CR⁸—CR⁸R⁹—, —CR⁸R⁹—CR⁸═, —CR⁸═CR⁹—, ═CR⁸—CR⁸═, —CR⁸R⁹—CR⁸R⁹—CR⁸R⁹—, ═CR⁸—CR⁸R⁹—CR⁸R⁹—, —CR⁸═CR⁸—CR⁸R⁹—, —CR⁸R⁹—CR⁸═CR⁸—, —CR⁸R⁹—CR⁸R⁹—CR⁸═, ═CR⁸—CR⁸═CR⁸—, —CR⁸═CR⁸—CR⁸═, and ═CR⁸—CR⁸R⁹—CR⁸═;
----E---- is chosen from —CR¹⁰R¹¹—, —CR¹⁰═, —CR¹⁰R¹¹—CR¹⁰R¹¹—, —CR¹⁰R¹⁰R¹¹—CR¹⁰═, —CR¹⁰═CR¹¹—, —CR¹⁰R¹¹—, —CR¹⁰R¹¹—CR¹⁰R¹¹—, —CR¹⁰═CR¹⁰—CR¹⁰R¹¹—, —CR¹⁰R¹¹—CR¹⁰═CR¹⁰—, —CR¹⁰R¹¹—CR¹⁰R¹¹—CR¹⁰═, and —CR¹⁰═CR¹⁰—CR¹⁰═;
the dotted lines in the 5-membered ring of formula (c) independently represent a single bond or a double bond; with the proviso there is at least one double bond between X⁹ and X¹⁰ or X¹⁰ and X¹¹;
the dotted lines in the 5-membered ring of formula (d) independently represent a single bond or a double bond; with the proviso there is at least one double bond between X¹² and X¹³ or X¹³ and X¹⁴;
the dotted lines in formula (f) independently represent a single bond or a double bond, with the proviso that when two double bonds are present, they are not adjacent to each other;
X¹, X², X³, X⁴, X⁵, X⁶, X⁷, X⁸, X¹⁸, X¹⁹, X²⁰ and X²¹ are each independently chosen from N and CR¹², and wherein two adjacent X¹-X⁴, X⁵-X⁸, and X¹⁸-X²¹ groups can each be CR¹² in which the two R¹² groups taken together form a fused ring structure chosen from methylenedioxy, ethylenedioxy group, difluoromethylenedioxy, and tetrafluoroethylenedioxy;
X⁹, X¹⁰, X¹¹, X¹², X¹³, and X¹⁴ are each independently chosen from S, O, N, NR¹², C(R¹²)₂, and CR¹²;
X¹⁵, X¹⁶ and X¹⁷ are each independently chosen from N and CR¹² wherein at least two of X¹⁵, X¹⁶ and X¹⁷ are not CR¹²;
X²² is chosen from N, C and CR¹² and X²³, X²⁴, X²⁵, and X²⁶ are each independently chosen from O, S, N, NR¹², C, CHR¹², C(R¹²)₂, and CR¹²; wherein at least two of X²², X²³, X²⁴, X²⁵, and X²⁶ are not chosen from C, CHR¹² and CR¹²;
R⁴, R⁵, R⁶, R⁷, R⁸, R⁹, R¹⁰, and R¹¹ are independently chosen from
   absent,
   H,
   carboxy,
   alkyl having 1 to 8,
   substituted alkyl having 1 to 8 carbon atoms and substituted with at least one group chosen from halogen, C₁₋₄-alkyl, C₁₋₄-alkoxy, and oxo,
   cycloalkyl having 3 to 12 carbon atoms,
   substituted cycloalkyl having 3 to 12 carbon atoms and substituted with at least one group chosen from halogen, C₁₋₄-alkyl, C₁₋₄-alkoxy, and oxo,
   cycloalkylalkyl having 4 to 12 carbon atoms, and
   substituted cycloalkylalkyl having 4 to 12 carbon atoms and substituted with at least one group chosen from halogen, C₁₋₄-alkyl, C₁₋₄-alkoxy, and oxo, or
R⁴ and R⁵ together form a cycloalkyl group chosen from 3 to 8 membered spiro cycloalkyl and 3 to 8 membered fused cycloalkyl, or
R⁶ and R⁷ together form a cycloalkyl group chosen from 3 to 8 membered spiro cycloalkyl and 3 to 8 membered fused cycloalkyl, or
R⁸ and R⁹ together form a cycloalkyl group chosen from 3 to 8 membered spiro cycloalkyl and 3 to 8 membered fused cycloalkyl, or
R¹⁰ and R¹¹ together form a cycloalkyl group chosen from 3 to 8 membered spiro cycloalkyl and 3 to 8 membered fused cycloalkyl, or
one or more of R⁴ and R⁵ and the carbon atom to which they are attached form a C(═O) group, or
one or more of R⁶ and R⁷ and the carbon atom to which they are attached form a C(═O) group, or
one or more of R⁸ and R⁹ and the carbon atom to which they are attached form a C(═O) group, or
one or more of R¹⁰ and R¹¹ and the carbon atom to which they are attached, in each case form a C(═O) group, R¹² is chosen from
   H,
   alkyl having up to 12 carbon atoms,
   substituted alkyl having up to 12 carbon atoms and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, or —NHCSNHR¹³ and wherein optionally one or more —CH₂CH₂— groups is replaced in each case by a group chosen from —CH═CH— and —C≡C—;
   cycloalkyl having 3 to 12 carbon atoms,
   substituted cycloalkyl having 3 to 12 carbon atoms and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from —CH═CH— and —C≡C—;
   cycloalkylalkyl having up to 12 carbon atoms,
   substituted cycloalkylalkyl having up to 12 carbon atoms and substituted and
   substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from CH═CH— and —═C—;
   heterocyclyl,
   heterocyclyl substituted with at least one group chosen from halogen, C₆₋₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   heteroaryl,
   heteroaryl substituted with at least one group chosen from halogen, C₆₋₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   aryl having 6 to 14 carbon atoms,
   substituted aryl having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
   arylalkyl having 7 to 16 carbon atoms,
   substituted arylalkyl having 7 to 16 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
   heteroarylalkyl wherein the heteroaryl portion has 5 to 10 ring atoms in which at least 1 ring atom is a heteroatom and the alkyl portion has 1 to 3 carbon atoms,
   substituted heteroarylalkyl wherein the heteroaryl portion has 5 to 10 ring atoms in which at least 1 ring atom is a heteroatom and the alkyl portion has 1 to 3 carbon atoms and wherein the heteroaryl portion is substituted by at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   aryloxy having 6 to 14 carbon atoms,
   substituted aryloxy having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy;
   heteroaryloxy having 5 to 10 ring atoms in which at least 1 ring atom is a heteroatom,
   substituted heteroaryloxy having 5 to 10 ring atoms in which at least 1 ring atom is a heteroatom, and substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   halogen, hydroxy, C₁₋₄-alkoxy, C₁₋₄-alkoxy-C₁₋₄-alkoxy, C₄₋₁₂-cycloalkylalkyloxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, —C₁₋₄-alkylthio, C₁₋₄-alkylsulphinyl, C₁₋₄-alkylsulphonyl, —SO₂NHR¹⁹, —SO₂NR¹⁸R¹⁹, —SO₂R²⁰, —NHSO₂R¹³, —NR¹³COR¹³, —CONHR¹³, —CONR¹³R¹⁹, —NHCONHR¹³, —OCONHR¹³, —NHCOOR¹³, —SCONHR¹³, —SCSNHR¹³, and —NHCSNHR¹³;
R¹³ is chosen from
   H,
   alkyl having 1 to 8 carbon atoms,
   substituted alkyl having 1 to 8 carbon atoms and substituted with at least one group chosen from halogen, C₁₋₄-alkyl, C₁₋₄-alkoxy, and oxo,
   cycloalkyl having 3 to 12 carbon atoms,
   substituted cycloalkyl having 3 to 12 carbon atoms and substituted with at least one group chosen from halogen, C₁₋₄-alkyl, C₁₋₄-alkoxy, and oxo;
   cycloalkylalkyl having 4 to 12 carbon atoms, and
   substituted cycloalkylalkyl having 4 to 12 carbon atoms and substituted with at least one group chosen from halogen, C₁₋₄-alkyl, C₁₋₄-alkoxy, and oxo;
R¹⁶ is chosen from
   aryl having 6 to 14 carbon atoms,
   substituted aryl having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
   heteroaryl having 5 to 10 ring atoms in which at least 1 ring atom is a heteroatom,
   substituted heteroaryl having 5 to 10 ring atoms in which at least 1 ring atom is a heteroatom and substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   heterocyclyl,
   substituted heterocyclyl substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
   carbocyclic, and
   substituted carbocyclic substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy;
R¹⁷ is chosen from
   alkyl having 1 to 12 carbon atoms,
   substituted alkyl having 1 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸ wherein optionally one or more —CH₂— groups is, in each case independently, replaced by —O—, —S—, or —NH— and wherein optionally one or more —CH₂CH₂— groups is replaced in each case by —CH═CH— or —C≡C—,
   cycloalkyl having 3 to 12 carbon atoms,
   substituted cycloalkyl having 3 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸, wherein optionally one or more —CH₂— groups is, in each case independently, replaced by —O—, —S—, or —NH— and wherein optionally one or more —CH₂CH₂— groups is replaced in each case by —CH═CH— or —C≡C—, cycloalkylalkyl,
   substituted cycloalkylalkyl substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸ wherein optionally one or more —CH₂— groups is, in each case independently, replaced by —O—, —S—, or —NH— and wherein optionally one or more —CH₂CH₂— groups is replaced in each case by —CH═CH— or —C≡C—,
   halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, ˜NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and NHCSNHR¹⁸;
R¹⁸ is chosen from
   H,
   alkyl having 1 to 8 carbon atoms, and
   substituted alkyl having 1 to 8 carbon atoms substituted with at least one group chosen from halogen, C₁₋₄-alkyl, C₁₋₄-alkoxy, and oxo;
R¹⁹ is chosen from
   H,
   alkyl having 1 to 8 carbon atoms,
   substituted alkyl having 1 to 8 carbon atoms and substituted with at least one group chosen from halogen, C₁₋₄-alkyl, C₁₋₄-alkoxy, and oxo,
   cycloalkyl having 3 to 10 carbon atoms,
   substituted cycloalkyl having 3 to 10 carbon atoms and substituted with at least one group chosen from halogen, C₁₋₄-alkyl, C₁₋₄-alkoxy, and oxo,
   cycloalkylalkyl having 4 to 12 carbon atoms,
   substituted cycloalkylalkyl having 4 to 12 carbon atoms and substituted with at least one group chosen from halogen, C₁₋₄-alkyl, C₁₋₄-alkoxy, and oxo,
   heteroaryl,
   heteroaryl substituted with at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
   heterocyclyl, and
   heterocyclyl substituted with at least one group chosen from halogen, C₆₋₁₄
   aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
R²⁰ is chosen from
   heterocyclyl, and
   heterocyclyl substituted by at least one group chosen from halogen, C₆₋₁₄-aryl-C₁₋₄-alkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
R²⁵ and R²⁶ are independently chosen from
   H,
   carboxy,
   alkyl having 1 to 8 carbon atoms,
   substituted alkyl having 1 to 8 carbon atoms and substituted with at least one group chosen from halogen, C₁₋₄-alkyl, C₁₋₄-alkoxy, and oxo,
   cycloalkyl having 3 to 12 carbon atoms,
   substituted cycloalkyl having 3 to 12 carbon atoms and substituted with at least one group chosen from halogen, C₁₋₄-alkyl, C₁₋₄-alkoxy, and oxo,
   cycloalkylalkyl having 4 to 12 carbon atoms,
   cycloalkylalkyl having 4 to 12 carbon atoms substituted with at least one group chosen from halogen, C₁₋₄-alkyl, C₁₋₄-alkoxy, and oxo, or
R²⁵ and R²⁶ together form a cycloalkyl group, spiro or fused, having 3 to 8 carbon atoms, or
R²⁵ and R²⁶ and the carbon atom to which they are attached form a C(═O) group; with the proviso that said compound of Formulas (I) and (II) is not chosen from
6,7-dimethoxy-4-(2-methyl-3,4-dihydroquinolin-1(2H)-yl)quinazoline;
4-(7-bromo-3,4-dihydroquinolin-1(2H)-yl)-6,7-dimethoxyquinazoline;
4-(5-bromo-3,4-dihydroquinolin-1(2H)-yl)-6,7-dimethoxyquinazoline;
6,7-dimethoxy-4-[7-(trifluoromethyl)-3,4-dihydroquinolin-1(2H)-yl]quinazoline;
6,7-dimethoxy-4-(6-methyl-3,4-dihydroquinolin-1- (2H)-yl)quinazoline;
4-(3,4-dihydroquinolin-1(2H)-yl)-6,7-dimethoxyquinazoline;
8-bromo-1-(6,7-dimethoxyquinazolin-4-yl)-2,3,4,5-tetrahydro-1H-1-benzazepine;
9-bromo-1-(6,7-dimethoxyquinazolin-4-yl)-2,3,4,5-tetrahydro-1H-1-benzazepine;
1-(6,7-dimethoxyquinazolin-4-yl)-2,3,4,5-tetrahydro-1H-1-benzazepine;
1-(6,7-dimethoxyquinazolin-4-yl)-1H-indole-3-carbaldehyde; 4-(1H-indol-1-yl)-6,7-dimethoxyquinazoline;
4-(1-H-benzotriazol-1-yl)-6,7-dimethoxyquinazoline;
4-(1-H-benzimidazol-1-yl)-6,7-dimethoxyquinazoline;
4-(1-H-indazol-1-yl)-6,7-dimethoxyquinazoline;
4-(5-fluorophenyl)-2-[4-(methylsulfonyl)phenyl)-1H-imidazol-4-yl)-6,7-dimethoxyquinazoline;
4-(1-cyclopropylmethyl)-4-(4-fluorophenyl)-1H-imidazol-5-yl)-6,7-dimethoxyquinazoline;
4-(5-(4-fluorophenyl)-3-phenyl-1H-1,2,4-triazol-1-yl)-6,7-dimethoxyquinazoline;
1-(6,7-dimethoxy-4quinazolinyl)-1H-pyrazole-3-amine;
N-[2-(6,7-dimethoxy-quinazolin-4-yl)-1,2,3,4-tetrahydro-isoquinolin-7-yl]-2,2-dimethyl-propionamide;
N-[2-(6,7-dimethoxy-quinazoline-4-yl)-1,2,3,4-tetrahydro-isoquinolin-7-yl]-acetamide;
6,7-dimethoxy-4-[8-(morpholine-4-sulfonyl)-3,4-dihydro-1H-isoquinolin-2-yl]-quinazoline;
6,7-dimethoxy-4-[8-(4-methyl-piperazine-1-sulfonyl)-3,4-dihydro-1H-isoquinolin-2-yl]-quinazoline;
4-(7,8-dimethoxy-3,4-dihydro-1H-isoquinolin-2-yl)-6-ethoxy-7-methoxy-quinazoline;
4-(6,7-dimethoxy-3,4-dihydro-1H-isoquinolin-2-yl)-6-ethoxy-7-methoxy-quinazoline;
4-(6,7-dimethoxy-3-methyl-3,4-dihydro-1H-isoquinolin-2-yl)-6-ethoxy-7-methoxy-quinazoline;
4-(3,4-dihydro-1H-isoquinolin-2-yl)-6-ethoxy-7-methoxy-quinazoline;
2-(6,7-dimethoxy-quinazolin-4-yl)-1,2,3,4-tetrahydro-isoquinolin-7-yl-amine; and
6,7-dimethoxy-4-(3-propyl-3,4-dihydro-1H-isoquinolin-2-yl)-quinazoline.

In certain embodiments, the methods of inhibiting PDE10 enzyme in a patient in need thereof comprising administering to said patient an effective amount of at least one chemical entity chosen from compounds of Formulas (I) and (II) are selective. In certain embodiments, the methods of inhibiting PDE10 enzyme in a patient in need thereof comprising administering to said patient an effective amount of at least one chemical entity chosen from compounds of Formulas (I) as described herein.

Also provided is the use of at least one chemical entity for the manufacture of a medicament for the treatment of a patient having a disease responsive to inhibition of PDE10 enzyme, wherein the at least one chemical entity is a chemical entity described herein.

Also provided is a method for the manufacture of a medicament for the treatment of a patient having a disease responsive to inhibition of PDE10 enzyme, comprising including in said medicament at least one chemical entity described herein.

Unless otherwise stated, the following terms used in the specification and claims are defined for the purposes of this Application and have the following meaning:

“Halogen” herein refers to F, Cl, Br, and I. In certain embodiments, halogens are F and Cl.

“Alkyl” means a straight-chain or branched-chain aliphatic hydrocarbon radical. Suitable alkyl groups include, but are not limited to, methyl, ethyl, propyl, isopropyl, butyl, sec-butyl, tert-butyl, pentyl, hexyl, heptyl, octyl, nonyl, decyl, undecyl, and dodecyl. Other examples of suitable alkyl groups include, but are not limited to, 1-, 2- or 3-methylbutyl, 1,1- , 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethylpropyl, trimethylpropyl, methylhexyl, dimethylpentyl, ethylpentyl, ethylmethylbutyl, dimethylbutyl, and the like.

These alkyl radicals can optionally have one or more —CH₂CH₂— groups replaced in each case by —CH═CH— or —C≡C— groups. Suitable alkenyl or alkynyl groups include, but are not limited to, 1-propenyl, 2-propenyl, 1-propynyl, 1-butenyl, 2-butenyl, 3-butenyl, 1-butynyl, 1,3-butadienyl, and 3-methyl-2-butenyl.

The alkyl groups also include cycloalkylalkyl in which the cycloalkyl portions have, unless otherwise specified, 3 to 8 carbon atoms, such as 4 to 6 carbon atoms and the alkyl portions have, e.g., 1 to 8 carbon atoms, such as 1 to 4 carbon atoms. Suitable examples include, but are not limited to, cyclopentylethyl and cyclopropylmethyl.

In the arylalkyl groups and heteroalkyl groups, “alkyl” refers to a divalent alkylene group having, e.g., 1 to 4 carbon atoms.

In the cases where alkyl is a substituent (e.g., alkyl substituents on aryl and heteroaryl groups) or is part of a substituent (e.g., in the alkylamino, dialkylamino, hydroxyalkyl, hydroxyalkoxy, alkylthio, alkylsulphinyl, and alkylsulphonyl substituents), the alkyl portion has, e.g., 1 to 12 carbon atoms, such as 1 to 8 carbon atoms, for example, 1 to 4 carbon atoms.

“Cycloalkyl” refers to monocyclic, bicyclic or tricyclic saturated hydrocarbon radical having, unless otherwise stated, 3 to 8 carbon atoms, such as 3 to 6 carbon atoms. Suitable cycloalkyl groups include, but are not limited to, cyclopropyl, cyclobutyl, cyclopentyl, cyclohexyl, cycloheptyl, cyclooctyl, and norbornyl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, and bicyclo[4.2.0]octyl.

Aryl, as a group or substituent per se or as part of a group or substituent, refers to an aromatic carbocyclic radical containing, e.g., 6 to 14 carbon atoms, such as 6 to 12 carbon atoms, for example, 6 to 10 carbon atoms. Suitable aryl groups include, but are not limited to, phenyl, naphthyl and biphenyl. Substituted aryl groups include the above-described aryl groups which are substituted one or more times by, for example, a group chosen from halogen, alkyl, hydroxy, alkoxy, nitro, methylenedioxy, ethylenedioxy, amino, alkylamino, dialkylamino, hydroxyalkyl, hydroxyalkoxy, carboxy, cyano, acyl, alkoxycarbonyl, alkylthio, alkylsulphinyl, alkylsulphonyl, phenoxy, and acyloxy (e.g., acetoxy) unless otherwise specified.

Aryloxy refer to aryl-O— groups wherein the aryl portion is accordance with the previous description, and thus has 6 to 14 carbon atoms, such as 6 to 10 carbon atoms. Suitable aryloxy groups include, but are not limited to, phenoxy and naphthoxy. Substituted aryloxy groups include the aryloxy groups which are substituted one or more times by, for example, a group chosen from halogen, alkyl, hydroxy, alkoxy, nitro, methylenedioxy, ethylenedioxy, amino, alkylamino, dialkylamino, hydroxyalkyl, hydroxyalkoxy, carboxy, cyano, acyl, alkoxycarbonyl, alkylthio, alkylsulphinyl, alkylsulphonyl, and phenoxy, unless otherwise specified.

Arylalkyl refers to an aryl-alkyl-radical in which the aryl and alkyl portions are in accordance with the previous descriptions. Suitable examples include, but are not limited to, 1-phenethyl, 2-phenethyl, phenpropyl, phenbutyl, phenpentyl, and naphthylenemethyl.

Heteroaryl groups refer to unsaturated heterocyclic groups having one or two rings and a total number of 5 to 10 ring atoms wherein at least one of the ring atoms is a heteroatom chosen from N, O and S. In certain embodiments, the heteroaryl group contains 1 to 4, e.g., 1 to 3, such as 1 or 2, hetero-ring atoms selected from N, O and S. Suitable heteroaryl groups include, but are not limited to, furyl, thienyl, pyrrolyl, pyrazolyl, imidazolyl, pyridyl, pyrimidinyl, indolyl, quinolinyl, naphthyridinyl, azaindolyl (e.g., 7-azaindolyl), 1,2,3,4,-tetrahydroisoquinolyl, thiazolyl, and the like. In certain embodiments, heteroaryl groups include, but are not limited to, 2-thienyl, 3-thienyl, 2-, 3- or 4-pyridyl, 2-, 3-, 4-, 5-, 6-, 7- or 8-quinolinyl, 7- azaindolyl, and 1-, 3-, 4-, 5-, 6-, 7- or 8-isoquinolinyl.

Substituted heteroaryl groups refer to the heteroaryl groups described above which are substituted in one or more places by, e.g., a group chosen from halogen, aryl, alkyl, alkoxy, cyano, halogenated alkyl (e.g., trifluoromethyl), nitro, oxo, amino, alkylamino, and dialkylamino, unless otherwise specified.

Heteroaryloxy refer to heteroaryl-O— groups wherein the heteroaryl portion is accordance with the previous description, and thus has one or two rings and a total number of 5 to 10 ring atoms wherein at least one of the ring atoms is chosen from N, O and S. In certain embodiments, the heteroaryl portion contains 1 to 4, e.g., 1 to 3, such as 1 or 2, hetero-ring atoms selected from N, O and S. The heteroaryl groups can be unsubstituted or substituted one or more times by, for example, a group chosen from halogen, aryl, arylalkyl, alkyl, hydroxy, alkoxy, nitro, oxo, amino, alkylamino, alkylamino, carboxy, cyano, alkoxycarbonyl, acyl, alkylthio, alkylsulphinyl, and alkylsulphonyl, unless otherwise specified.

Heterocycles are non-aromatic, saturated or partially unsaturated, cyclic groups having 5 to 10 ring atoms and containing at least one hetero ring atom selected from N, S, and O. In certain embodiments, heterocycles contains 1 to 4, e.g., 1 to 3, such as 1 or 2, hetero-ring atoms selected from N, O and S. Suitable heterocycles include, but are not limited to, 3-tetrahydrofuranyl, piperidinyl, imidazolinyl, imidazolidinyl, pyrrolinyl, pyrrolidinyl, morpholinyl, piperazinyl, oxazolidinyl, and indolinyl.

Heteroarylalkyl refers to a heteroaryl-alkyl-group wherein the heteroaryl and alkyl portions are in accordance with the previous discussions. Suitable examples include, but are not limited to, pyridylmethyl, thienylmethyl, pyrimidinylmethyl, pyrazinylmethyl, isoquinolinylmethyl, pyridylethyl and thienylethyl.

Carbocyclic structures are non-aromatic monocyclic or bicyclic structures containing 5 to 14 carbon atoms, such as 6 to 10 carbon atoms, wherein the ring structure(s) optionally contain at least one C═C bond. Suitable examples include, but are not limited to, cyclopentenyl, cyclohexenyl, tetrahydronaphthenyl, and indan-2-yl.

Acyl refers to alkanoyl (—COR) radicals having 2 to 4 carbon atoms. Suitable acyl groups include, but are not limited to, formyl, acetyl, propionyl, and butanoyl.

Substituted radicals have, e.g., 1 to 3 substituents, such as 1 or 2 substituents.

“Optionally” means that the subsequently described event or circumstance may but need not occur, and that the description includes instances where the event or circumstance occurs and instances in which it does not. For example, “optionally substituted” alkyl, cycloalkyl, or cycloalkylalkyl means an alkyl, cycloalkyl, or cycloalkylalkyl group, respectively, which is unsubstituted or substituted with one or more groups wherein those one or more groups are as described further herein.

One of ordinary skill in the art will recognize that some of the compounds of Formulas I and II can exist in different geometrical isomeric forms. In addition, some of the compounds possess one or more asymmetric atoms and are thus capable of existing in the form of optical isomers, as well as in the form of racemic or nonracemic mixtures thereof, and in the form of diastereomers and diastereomeric mixtures inter alia. All of these compounds, including cis isomers, trans isomers, diastereomeric mixtures, racemates, nonracemic mixtures of enantiomers, substantially pure enantiomers, and pure enantiomers, fall within the scope of the chemical entities described herein. Substantially pure enantiomers contain no more than 5% w/w of the corresponding opposite enantiomer, such as no more than 2%, for example, no more than 1%.

The optical isomers can be obtained by resolution of the racemic mixtures according to conventional processes, for example, by the formation of diastereomeric salts using an optically active acid or base or formation of covalent diastereomers.

Examples of appropriate optically active acids include, but are not limited to, tartaric, diacetyltartaric, dibenzoyltartaric, ditoluoyltartaric and camphorsulfonic acid. Mixtures of diastereomers can be separated into their individual diastereomers on the basis of their physical and/or chemical differences by methods known to those skilled in the art, for example, by chromatography or fractional crystallization. The optically active bases or acids are then liberated from the separated diastereomeric salts.

A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of Formulas I and II can likewise be obtained by utilizing optically active starting materials in chiral syntheses processes under reaction conditions which do not cause racemization.

In addition, one of ordinary skill in the art will recognize that the compounds can be used in different enriched isotopic forms, e.g., enriched in the content of ²H, ³H, ¹¹C, ¹³C and/or ¹⁴C. In some embodiments, the compounds are deuterated. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp. CAN 133:68895 AN 2000:473538 CAPLUS; Kabalka, George W.; Varma, Rajender S. The Synthesis of Radiolabeled Compounds via Organometallic Intermediates, Tetrahedron, 1989, 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem., 1981, 64(1-2), 9-32. CODEN: JRACBN ISSN:0022-4081, CAN 95:76229 AN 1981:476229 CAPLUS.

The chemical entities described herein include free base forms, as well as pharmaceutically acceptable salts or prodrugs of all the compounds for which salts or prodrugs can be prepared. Pharmaceutically acceptable salts include those obtained by reacting the main compound, functioning as a base, with an inorganic or organic acid to form a salt, for example, but not limited to, salts of hydrochloric acid, sulfuric acid, phosphoric acid, methanesulfonic acid, camphorsulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of the compounds with the appropriate inorganic or organic acid via any of a number of known methods. Alternatively, alkali and alkaline earth metal salts may be prepared by reacting the compounds described herein with the appropriate base via a variety of known methods.

The following are further non-limiting examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates. For example, the pharmaceutically acceptable salt can be a hydrochloride, a hydroformate, hydrobromide, or a maleate.

In certain embodiments, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of Formulas I and II can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of Formulas I and II can exist in different solvate forms. Solvates may also form when solvent molecules are incorporated into the crystalline lattice structure of the compound molecule during the crystallization process. For example, suitable solvates include hydrates, e.g., monohydrates, dihydrates, sesquihydrates, and hemihydrates.

(1) In certain embodiments, R¹ and R² are alkyl. In certain embodiments, R¹ and R² are methyl. In certain embodiments, R¹ is chosen from ethyl, propyl, and butyl and R² is methyl.

(2) In certain embodiments, R¹ and R² are haloalkyl. In certain embodiments, R¹ and R² are independently chosen from trifluoromethyl and difluoromethyl.

(a) Within the above embodiments (1) and (2), including the subgroups contained therein, in certain embodiments, R³ is chosen from 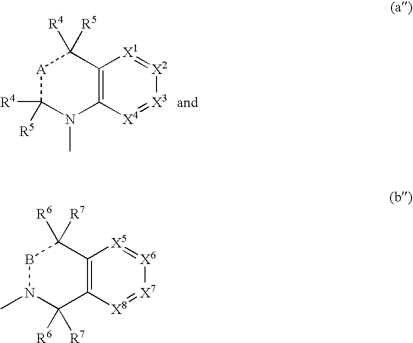
in which X¹, X², X³, X⁴, X⁵, X⁶, X⁷, and X⁸ are each CR¹², and R¹² is chosen from H, halogen, COOH, CH₂OCH₃, CONH-cyclopropyl, CONHCH₂-cyclopropyl, OH, OCH₃, OC₂H₅, CH₂OH, OCH₂CH₂OH, and OCH₂CH₂OCH₃.

Within the above embodiments (1) and (2), including the subgroups contained therein, in certain embodiments, R³ is

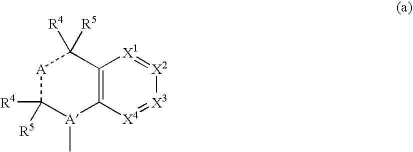

In certain embodiments in group (a), A′ is —N—.

In certain embodiments in group (a), ----A---- is chosen from a double bond and —CR⁴R⁵. In certain embodiments in group (a), ----A---- is chosen from a double bond and —CR⁴R⁵— and A′ is —N—.

In certain embodiments in group (a), A′ is —N—, ----A---- is —CR⁴R⁵— and X¹-X⁴ are CH, then R⁴ and R⁵ are not all H, and if one of the R⁴ and R⁵ groups is methyl then at least one of the remaining R⁴ and R⁵ groups is other than H. In certain embodiments in group (a), ----A---- is —CR⁴R⁵—, and each of the R⁴ and R⁵ groups is absent, H, alkyl, COOH, or one set of R⁴ and R⁵ together with the carbon to which they are attached form a C(═O) group.

In certain embodiments in group (a), A′ is —N—, ----A---- is —CH₂—, and all R⁴ and R⁵ are H, then at least one of X¹-X⁴ is CR² in which R¹² is not chosen from H, halogen, alkyl, and haloalkyl. In certain embodiments in group (a), A′ is —N—, ----A---- is —CH₂—, all R⁴ and R⁵ are H, and at least one of X¹-X⁴ is CR¹² in which R¹² is chosen from hydroxy, C₁₋₄-alkoxy, C₁₋₄-alkoxy-C₁₋₄-alkoxy (e.g., methoxyethoxy), C₄₋₁₂-cycloalkylalkyloxy (e.g., O-cyclopropylmethyl), aryloxy (e.g., phenoxy), halogenated C₁₋₄ alkoxy, and C₂₋₄-hydroxyalkoxy (e.g., OCH₂CH₂OH). In certain embodiments in group (a), R¹² is chosen from C₁₋₄ alkoxy and alkyloxyalkoxy.

In certain embodiments in group (a), A′ is —N—, -----A----- is —CR⁴R⁵—CR⁴R⁵, and all R⁴ and R⁵ are H, then at least one of X¹-X⁴ is CR¹² in which R¹² is not chosen from H, alkyl, and halogen. In certain embodiments in group (a), A′ is —N—, -----A----- is —CR⁴R⁵—CR⁴R⁵, and all R⁴ and R⁵ are H, then at least one of X¹-X⁴ is CR¹² in which R¹² is not chosen from H, CH₃, and halogen. In certain embodiments in group (a), A′ is —N—, -----A------ is —CR⁴R⁵—CR⁴R⁵—, and all R⁴ and R⁵ are H, then at least one of X¹-X⁴ is CR¹¹ in which R¹² is not chosen from H and halogen.

In certain embodiments in group (a), A′ is —N—, -----A----- is a double bond, and all R⁴ and R⁵ are H or are absent, then at least one of X¹-X⁴ is CR¹² in which R¹² is not chosen from H and CHO. In certain embodiments in group (a), A′ is —N—, -----A----- is a double bond, and all R⁴ and R⁵ are H or are absent, then at least one of X¹-X⁴ is CR¹² in which R¹² is not chosen from H and COR³. In certain embodiments in group (a), X¹-X⁴ are each CR¹², R¹² is chosen from H and alkyl, A′ is —N—, and -----A----- is a double bond, then R⁴ and R⁵ are other than CHO. In certain embodiments in group (a), X¹-X⁴ are each CR¹², R¹² is chosen from H and alkyl, A′ is —N—, and -----A----- is a double bond, then R⁴ and R⁵ are other than COR¹³. In certain embodiments in group (a), X¹-X⁴ are each CR¹², A′ is —N—, and -----A----- is a double bond, then at least one R¹² is not chosen from H, halogen, CN, C₁₋₄ alkyl, nitro, NH₂, NH(C₁₋₄ alkyl), N(C₁₋₄ alkyl)₂, COOH, COO(C₁₋₄ alkyl), CHO, CONH₂, CONH(C₁₋₄ alkyl), CON(C₁₋₄ alkyl)₂, O(C₁₋₄ alkyl), phenoxy, and CH(OC₁₋₄ alkyl)₂. In certain embodiments in group (a), X¹-X⁴ are independently chosen from CH and CCH₃, A′ is —N—, and -----A----- is a double bond, then R⁴ and R⁵ are other than CHO. In certain embodiments in group (a), X¹-X⁴ are independently chosen from CH and CCH₃, A′ is —N—, and -----A----- is a double bond, then R⁴ and R⁵ are other than COR³. In certain embodiments in group (a), X¹-X⁴ are each CH, A′ is —N—, and -----A----- is a double bond, then R⁴ and R⁵ are other than CHO. In certain embodiments in group (a), X¹-X⁴ are each CH, A′ is —N—, and -----A----- is a double bond, then R⁴ and R⁵ are other than COR¹³.

In certain embodiments in group (a), one set of R⁴ and R⁵ together with the carbon to which they are attached form a C(═O) group.

In certain embodiments in group (a), R⁴ and R⁵ are independently chosen from absent, H, carboxy, and CH₃.

(b) Within the above embodiments (1) and (2), including the subgroups contained therein, in certain embodiments, R³ is

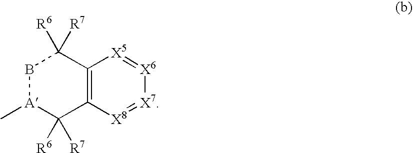

In certain embodiments in group (b), A′ is —N—.

In certain embodiments in group (b), ----B---- is chosen from a single bond and CR⁵R⁶—. In certain embodiments in group (b), ----B---- is chosen from a single bond and —CR⁵R⁶— and A′ is —N—.

In certain embodiments in group (b), R³ is an isoquinoline group of the formula (b1), 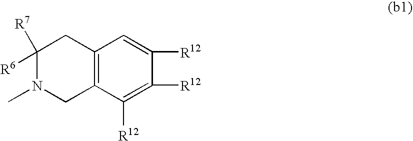
then R⁶ and R⁷ are each not alkyl, the R¹² group attached to the 8-position of the isoquinoline is not chosen from alkoxy and —SO₂R²¹ in which R²⁰ is chosen from morpholino, substituted morpholino, piperazino, and substituted piperazino, the R¹² group attached to the 7-position of the isoquinoline is not chosen from alkoxy, amino, alkylamino, and —NR¹³COR¹³ in which R¹³ in each case is chosen from H and alkyl, the R¹² group attached to the 6-position of the isoquinoline is not alkoxy, and R⁶, R⁷, and the three R¹² are not all H.

In certain embodiments in group (b), R³ is an isoquinoline group of the formula (b1), 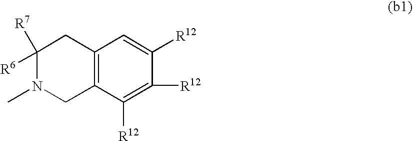
then R⁶ and R⁷ are each not alkyl, the R¹² group attached to the 8-position of the isoquinoline is not chosen from alkoxy and —SO₂R²⁰, the R¹² group attached to the 7-position of the isoquinoline is not chosen from alkoxy, amino, alkylamino, and —NR¹³COR¹³, the R¹² group attached to the 6-position of the isoquinoline is not alkoxy, and R⁶, R⁷, and the three R¹² are not all H.

In certain embodiments in group (b), R³ is an isoquinoline group of the formula (b1), 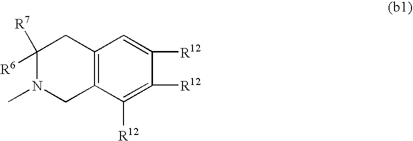
then R⁶ and R⁷ are each not alkyl, each of the R¹² groups is not alkoxy, amino, alkylamino, —SO₂R²⁰ in which R²⁰ is chosen from morpholino, substituted morpholino, piperazino, substituted piperazino, and —NR¹³COR¹³ in which R¹³ in each case is chosen from H and alkyl, and R⁶, R⁷, and the three R¹² are not all H.

In certain embodiments in group (b), R³ is an isoquinoline group of the formula (b1), 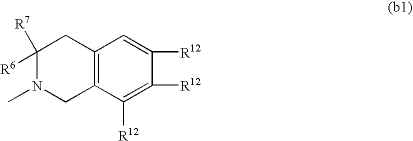
then R⁶ and R⁷ are each not alkyl, each of the R¹² groups is not chosen from alkoxy, —SO₂R²⁰,— and —NR¹³COR¹³, and R⁶, R⁷, and the three R¹² are not all H.

In certain embodiments in group (b), R³ is an isoquinoline group of the formula (b2), 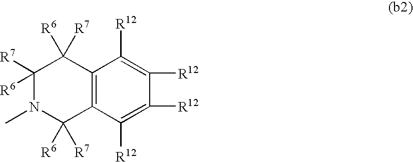
then at least one R¹² is not chosen from H, alkoxy, amino, alkylamino, —COR¹³, —COOR, —SO₂NHR¹³, —SO₂NHR¹⁹, —SO₂NR¹⁸R¹⁹, —SO₂R²⁰—NHSO₂R¹³, —NR¹³COR¹³—CONHR¹³, —CONR¹³R¹⁹, CONH-cycloalkyl, —NHCONHR¹³, and —NHCOOR¹³, and at least two R¹² are not alkoxy, and the R⁶, R⁷, and R¹² groups are not all H.

In certain embodiments in group (b), R⁶ and R⁷ are independently chosen from absent, H, carboxy, and CH₃.

In certain embodiments in group (b), R³ is an isoquinoline group chosen from 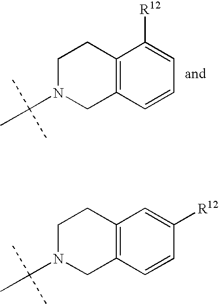
where the isoquinoline ring can optionally be further substituted with R¹². In certain embodiments in group (b), R¹² is optionally substituted heteroaryl. In certain embodiments in group (b), R¹² is chosen from optionally substituted saturated heterocyclyl and optionally substituted partially saturated heterocyclyl, and in certain embodiments in group (b), R¹² is chosen from optionally substituted piperazinyl, optionally substituted piperidinyl, and optionally substituted morpholinyl.

In certain embodiments in group (b), R³ is an isoquinoline group chosen from 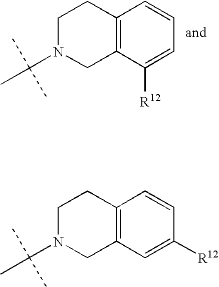
where the isoquinoline ring can optionally be further substituted with R¹². In certain embodiments in group (b), R¹² is chosen from hydroxy-C₁₋₄-alkoxy (e.g., hydroxyethyoxy) and C₁₋₄-alkoxy-C₁₋₄-alkoxy (e.g., methoxyethoxy). In certain embodiments in group (b), R¹² is optionally substituted heteroaryl. In certain embodiments in group (b), R¹² is chosen from optionally substituted saturated heterocyclyl and optionally substituted partially saturated heterocyclyl, and in certain embodiments in group (b), R¹² is chosen from optionally substituted piperazinyl, optionally substituted piperidinyl, and optionally substituted morpholinyl.

In certain embodiments in group (b), R³ is chosen from:

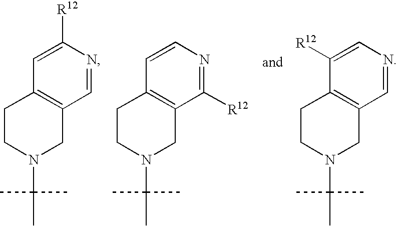

In certain embodiments in group (b), R¹² is chosen from alkyl, C₁₋₄alkoxy, halogenated C₁₋₄alkoxy, cycloalkyl, aryl, heteroaryl, heterocyclyl, C₁₋₄ alkylamino, di-C₁₋₄-alkylamino, —COR¹³, C₁₋₄-alkylthio, C₁₋₄-alkylsulphinyl, C₁₋₄-alkylsulphonyl, —NR¹³ COR¹³, —CONHR¹³, —CONR¹³R¹⁹, —NHSO₂R¹³, —SO₂NHR¹⁹, and —SO₂NR¹⁸R¹⁹ and wherein the ring in R¹² is optionally substituted. In certain embodiments in group (b), R¹² is chosen from cycloalkyl, aryl, heteroaryl, and heterocyclyl, each of which is optionally substituted. In certain embodiments in group (b), R¹³ is chosen from hydrogen and alkyl.

In certain embodiments in group (b), R³ is a group of formula:

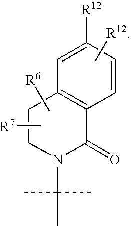

In certain embodiments in group (b), R³ is chosen from:

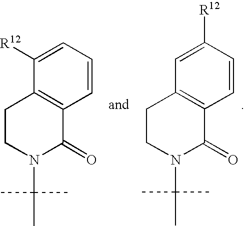

The isoquinoline ring can optionally be further substituted with R¹². In certain embodiments in group (b), R¹² is chosen from optionally substituted heteroaryl. In certain embodiments in group (b), R¹² is a heterocyclyl group chosen from optionally substituted saturated heterocyclyl and optionally substituted partially saturated heterocyclyl groups. In certain embodiments in group (b), R¹² is chosen from optionally substituted piperazinyl, optionally substituted piperidinyl, and optionally substituted morpholinyl.

(c) Within the above embodiments (1) and (2), including the subgroups contained therein, in certain embodiments, R³ is

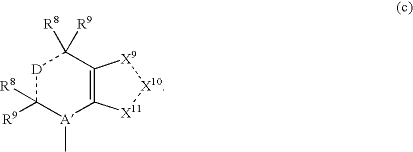

In certain embodiments in group (c), A′ is —N—.

(d) Within the above embodiments (1) and (2), including the subgroups contained therein, in certain embodiments, R³ is

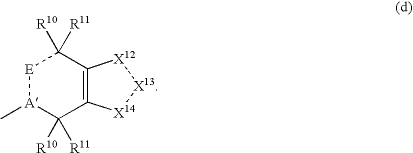

In certain embodiments in group (d), A′ is —N—.

In certain embodiments in group (d), R³ is chosen from

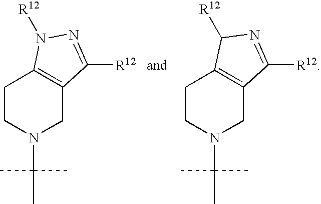

In certain embodiments in group (d), R¹² is chosen from hydrogen, halo, alkyl, C₁₋₄alkoxy, halogenated C₁₋₄alkoxy, cycloalkyl, aryl, heteroaryl, heterocyclyl, C₁₋₄ alkylamino, di-C₁₋₄-alkylamino, —COR¹³, C₁₋₄-alkylthio, C₁₋₄-alkylsulphinyl, C₁₋₄-alkylsulphonyl, —NR¹³COR¹³, —CONHR¹³, —CONR¹³R¹⁹, —NHSO₂R¹³, —SO₂NHR¹⁹, and —SO₂NR¹⁸R¹⁹ and wherein the ring in R¹² is optionally substituted. In certain embodiments in group (d), R¹² is chosen from phenyl, heteroaryl, and heterocyclyl, each of which is optionally substituted. In certain embodiments in group (d), R¹³ is chosen from hydrogen and alkyl.

In certain embodiments in group (d), R³ is

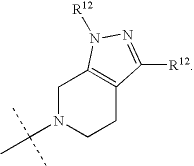

In certain embodiments in group (d), R¹² is chosen from hydrogen, halo, alkyl, C₁₋₄-hydroxyalkyl, C₁₋₄alkoxy, halogenated C₁₋₄alkoxy, cycloalkyl, aryl, heteroaryl, heterocyclyl, C₁₋₄ alkylamino, di-C₁₋₄-alkylamino, —COR¹³, —COOR¹³, C₁₋₄-alkylthio, C₁₋₄-alkylsulphinyl, C₁₋₄-alkylsulphonyl, —NR¹³COR¹³, —CONHR¹³R¹³, —CONR¹³R¹⁹, —NHSO₂R¹³, —SO₂NHR¹⁹, and —SO₂NR¹⁸R¹⁹ and wherein the ring in R¹² is optionally substituted. In certain embodiments in group (d), R¹² is chosen from phenyl, heteroaryl, and heterocyclyl, each of which is optionally substituted. In certain embodiments in group (d), R¹³ is chosen from hydrogen and alkyl.

(e) Within the above embodiments (1) and (2), including the subgroups contained therein, in certain embodiments, R³ is

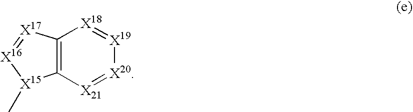

In certain embodiments in group (e), X¹⁵-X¹⁷ are each N and at least one of X¹⁸, X¹⁹, X²⁰, and X²¹ is other than CH. In certain embodiments in group (e), X¹⁵ and X¹⁶ are N and X¹⁷ is CR¹² (e.g., CH). In certain embodiments in group (e), X¹⁵ is CR¹² (e.g., CH) and X¹⁶ and X¹⁷ are N. In certain embodiments in group (e), X¹⁵ and X¹⁷ are N and X¹⁶ is CH and at least one of X¹¹, X¹⁹, X²⁰, and X²¹ is other than CH. In certain embodiments in group (e), X¹⁵ and X¹⁶ are N and X¹⁷ is CH, and at least one of X¹⁸, X¹⁹, X²⁰, and X²¹ is other than CH. In certain embodiments in group (e), X¹⁵-X¹⁷ are each N. In certain embodiments in group (e), X¹⁵ and X¹⁷ are N and X¹⁶ is CH. In certain embodiments in group (e), X¹⁵ and X¹⁶ are N and X¹⁷ is CH, and at least one of X¹⁸, X¹⁹, X²⁰, and X²¹ is other than CH.

In certain embodiments in group (e), R³ is chosen from

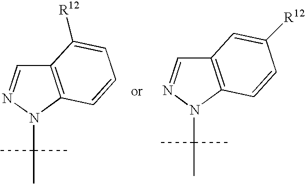

In certain embodiments in group (e), R¹² is chosen from cycloalkyl, cycloalkylalkyl, arylalkyl, heteroarylalkyl, aryl, heteroaryl, heterocyclyl, C₁₋₄ alkylamino, di-C₁₋₄-alkylamino, —COR¹³, C₁₋₄-alkylthio, C₁₋₄-alkylsulphinyl, C₁₋₄-alkylsulphonyl, —NR¹³COR¹³, —CONHR¹⁸, —CONR¹³R¹⁹, —NHSO₂R¹³, —SO₂NHR¹⁸, and —SO₂NR¹⁸R¹⁹, each of which is optionally substituted. In certain embodiments in group (e), R¹³ is chosen from hydrogen and alkyl. In certain embodiments in group (e), R¹² is chosen from phenyl, heteroaryl, and heterocyclyl, each of which is optionally substituted. In certain embodiments in group (e), R³ is: 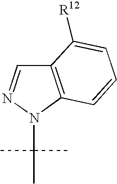
where R¹² is chosen from phenyl, heteroaryl, a five-membered heterocyclyl group which is chosen from saturated and partially saturated five-membered heterocyclyl groups, and a six-membered heterocyclyl group which is chosen from saturated and partially saturated six-membered heterocyclyl groups, each of which is optionally substituted. In certain embodiments in group (e), R³ is: 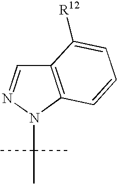
where R¹² is chosen from morpholin-4-yl, piperazin-1-yl, and pyridinyl, each of which is optionally substituted.

In certain embodiments in group (e), R³ is a group of formula:

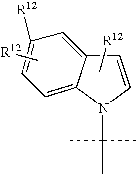

In certain embodiments in group (e), R³ is a group of formula:

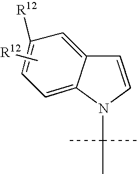

In certain embodiments in group (e), one occurrence of R¹² is chosen from hydrogen, halo, alkyl, C₁₋₄alkoxy, halogenated C₁₋₄alkoxy, cyano, amino, C₁₋₄ alkylamino, di-C₁₋₄-alkylamino, —COR¹³, C₁₋₄-alkylthio, C₁₋₄-alkylsulphinyl, C₁₋₄-alkylsulphonyl, —NR¹³COR¹³, —CONHR¹³, —CONR¹³R¹⁹, —NHSO₂R¹³, —SO₂NHR¹⁹, and —SO₂NR¹⁸R¹⁹, each of which is optionally substituted and the other occurrence of R¹² is chosen from cycloalkyl, aryl, heteroaryl, and heterocyclyl, and wherein the ring in R¹² is optionally substituted. In certain embodiments in group (e), that other occurrence of R¹² is chosen from aryl, heteroaryl, and heterocyclyl, each of which is optionally substituted. In certain embodiments in group (e), R¹³ is chosen from hydrogen and alkyl

(f) Within the above embodiments (1) and (2), including the subgroups contained therein, in certain embodiments, R³ is

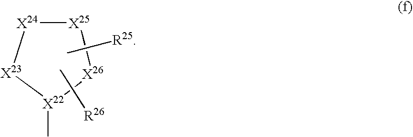

In certain embodiments in group (f), at least one of X²²-X²⁶ is CR¹² and at least one R¹² is is not chosen from amino, cycloalkylalkyl, substituted phenyl, and phenyl. In certain embodiments in group (f), two of X²²-X²⁵ are independently chosen from N and NR¹² and the rest of X²²-X²⁵ are independently chosen from C and CR¹². In certain embodiments in group (f), at least one of X²²-X²⁶ is CR¹² and at least one R¹² is not chosen from amino, C₁₋₄ alkylamino, di-C₁₋₄-alkylamino, cycloalkylalkyl, substituted phenyl, and phenyl. In certain embodiments in group (f), at least one of X²²-X²⁶ is CR¹² and at least one
R¹ is not chosen from amino, methylamino, dimethylamino, cycloalkylalkyl, substituted phenyl, and phenyl. In certain embodiments in group (f), the ring of formula (f) contains no double bonds or two non-adjacent double bonds. In certain embodiments in group (f), X²² and X²⁵ are independently chosen from N and NR¹² and the others are independently chosen from C and CR¹². In certain embodiments in group (f), X²⁴ and X²⁵ are independently chosen from N and NR¹² and the others are independently chosen from C and CR¹² (e.g., CH).

In certain embodiments in group (f), R³ is a group of formula: 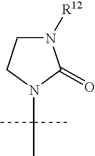
where R¹² is optionally substituted arylalkyl. In certain embodiments in group (f), R³ is optionally substituted benzyl.

In certain embodiments in group (f), R³ is a group of formula: 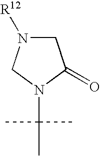
where R¹² is optionally substituted arylalkyl. In certain embodiments in group (f), R¹² is optionally substituted benzyl.

In certain embodiments in group (f), R³ is a group of formula:

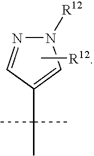

In certain embodiments in group (f), one R¹² is chosen from hydrogen and alkyl and the other is chosen from aryl, heteroaryl, arylalkyl, heteroarylalkyl, and heterocyclyl, each of which is optionally substituted. In certain embodiments in group (f), R¹² is optionally substituted arylalkyl. In certain embodiments in group (f), R¹² is optionally substituted benzyl. In certain embodiments in group (f), R¹² is optionally substituted heteroaryl. In certain embodiments in group (f), R¹² is heterocyclyl optionally substituted with a group chosen from optionally substituted phenyl and optionally substituted heteroaryl. In certain embodiments in group (f), R³ is a group of formula: 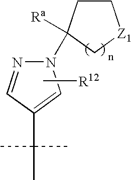
where R¹² is chosen from hydrogen and alkyl, n is chosen from 1, 2, and 3; Z, is chosen from —O—, —NH— and —N—, -alkyl-; and R^{a} is chosen from optionally substituted phenyl and optionally substituted heteroaryl. In certain embodiments in group (f), R¹² is hydrogen. In certain embodiments in group (f), R^{a} is optionally substituted phenyl.

(g) Within the above embodiments (1) and (2), including the subgroups contained therein, in certain embodiments, R³ is CHR¹⁶R¹⁷.

In certain embodiments in group (g), R¹⁶ is 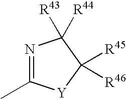
wherein
Y is chosen from NR⁴⁷, O and S; and
R⁴³, R⁴⁴, R⁴⁵, R⁴⁶ and R⁴⁷ are each independently chosen from H, halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl.

In certain embodiments in group (g), R¹⁶ is 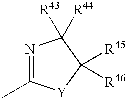
wherein
Y is chosen from NR⁴⁷ and O, and
R⁴³, R⁴⁴, R⁴⁵, R⁴⁶ and R⁴⁷ are each independently chosen from H, halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl.

In certain embodiments in group (g), R⁴³ and R⁴⁴ are independently chosen from H, CH₃ and phenyl. In certain embodiments in group (g), R⁴³ and R⁴⁴ are independently chosen from H and CH₃. In certain embodiments in group (g), R⁴³ and R⁴⁴ are H.

In certain embodiments in group (g), R⁴⁶ is chosen from cyclopropyl, benzyl, and cyclopropylmethyl.

In certain embodiments in group (g), R¹⁷ is CN.

(h) Within the above embodiments (1) and (2), including the subgroups contained therein, in certain embodiments, R³ is chosen from: 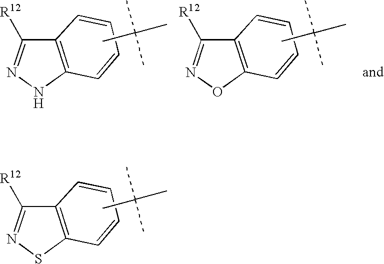
where: R¹² is chosen from cycloalkyl, cycloalkylalkyl, arylalkyl, heteroarylalkyl, aryl, heteroaryl, heterocyclyl, C₁₋₄ alkylamino, di-C₁₋₄-alkylamino, —COR¹³, C₁₋₄-alkylthio, C₁₋₄-alkylsulphinyl, C₁₋₄-alkylsulphonyl, —NR¹³COR¹³, —CONHR¹³, —CONR¹³R¹⁹, —NHSO₂R¹³, —SO₂NHR¹⁹, and —SO₂NR¹⁸R¹⁹, each of which is optionally substituted. In certain embodiments in group (h), R¹³ is chosen from hydrogen and alkyl. In certain embodiments in group (h), R³ is 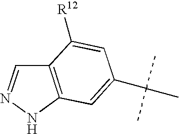
wherein R¹² is chosen from heteroaryl, phenyl and heterocyclyl, each of which is optionally substituted and wherein the hydrogen in the —NH— group in the ring is optionally substituted. In certain embodiments in group (h), R³ is 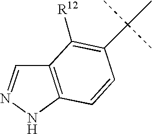
wherein R¹² is chosen from heteroaryl, phenyl and heterocyclyl ring, each of which is optionally substituted and wherein the hydrogen in the —NH— group in the ring is optionally substituted.

In certain embodiments, the compound of Formulas (I) and (II) is chosen from the compounds set forth in Table 1.

TABLE I
Structure/Name

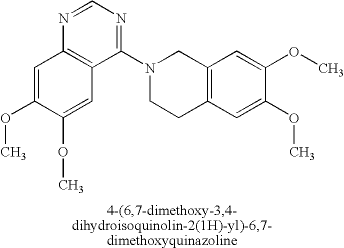

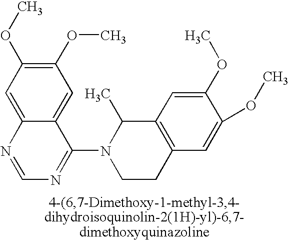

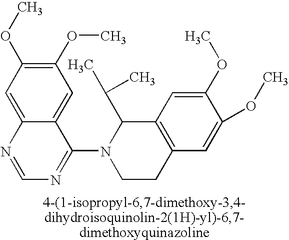

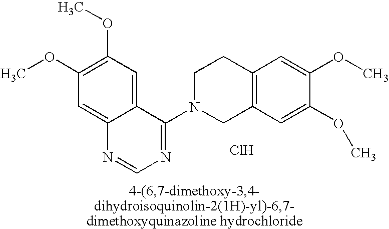

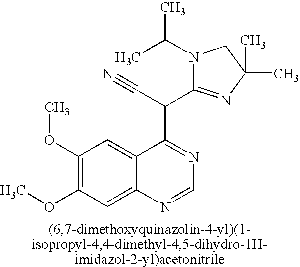

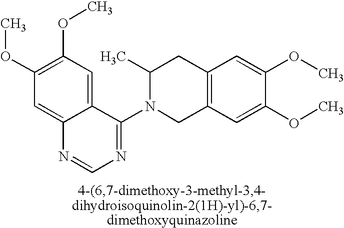

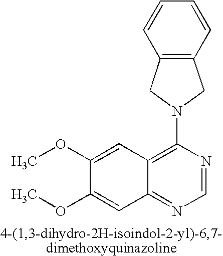

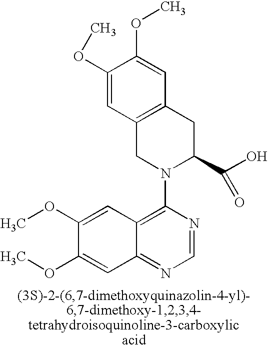

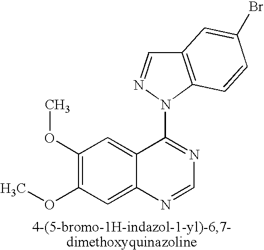

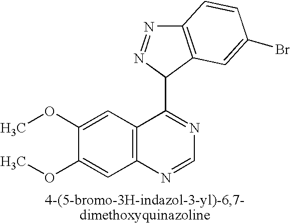

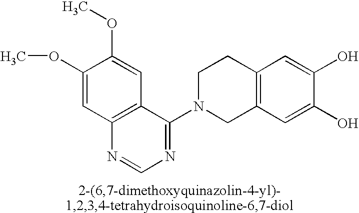

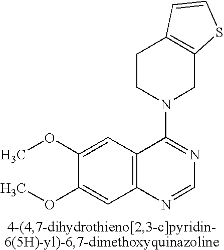

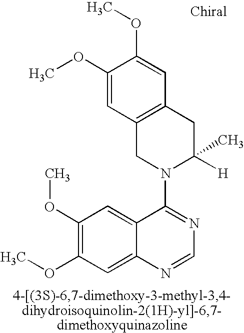

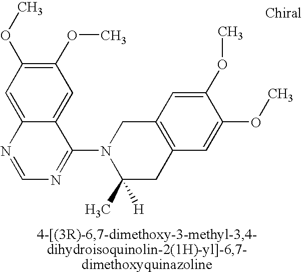

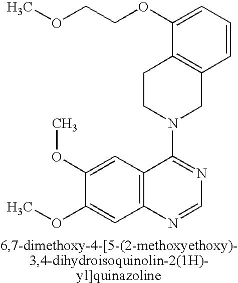

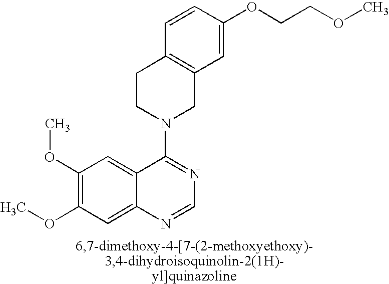

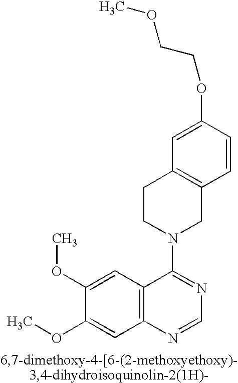

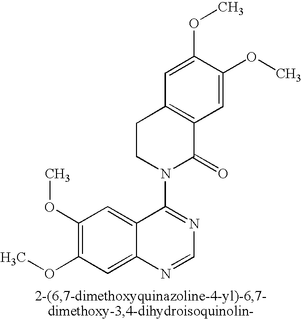

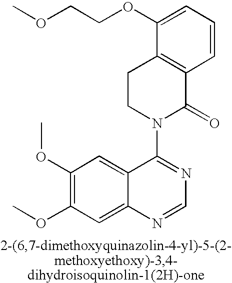

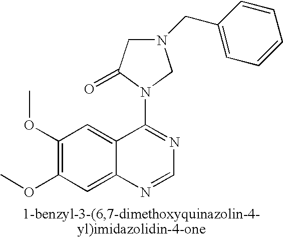

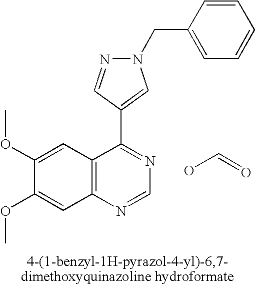

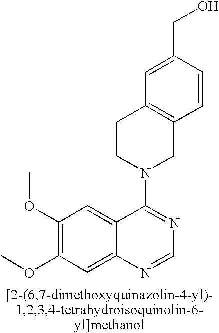

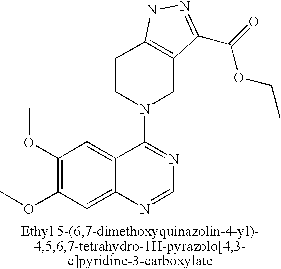

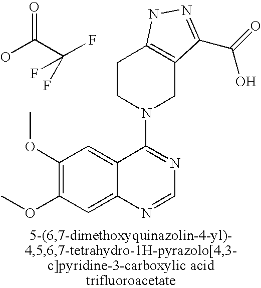

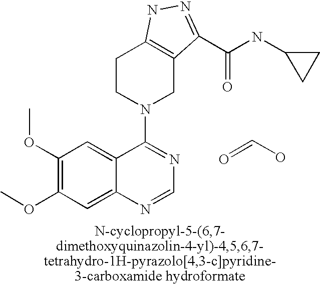

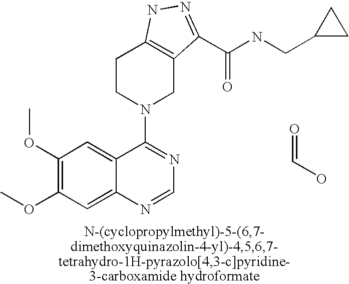

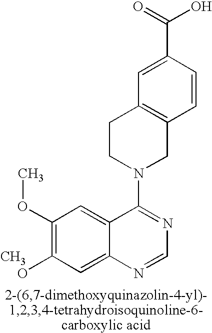

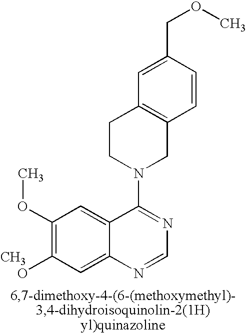

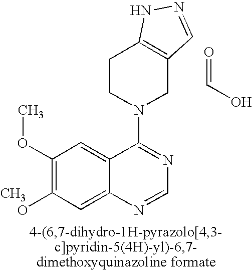

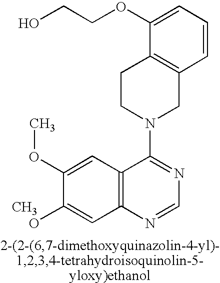

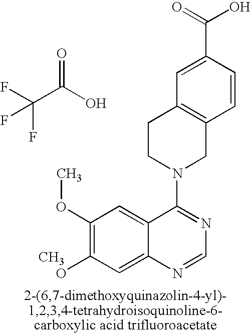

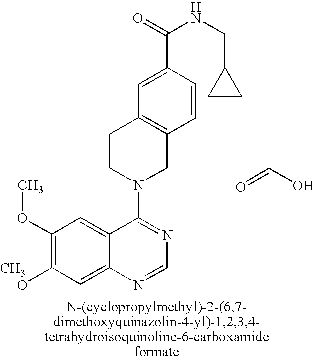

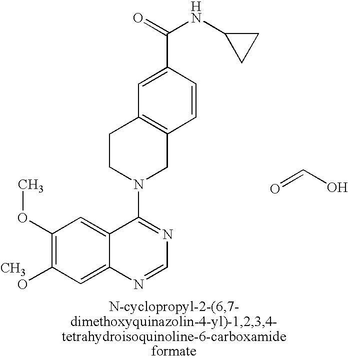

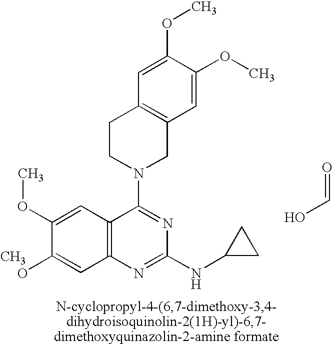

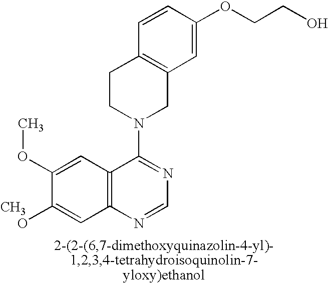

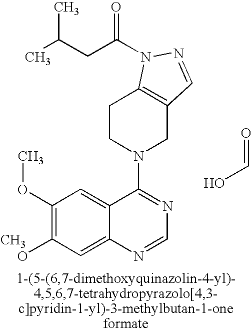

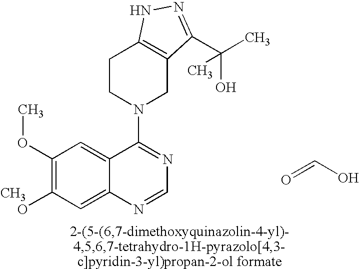

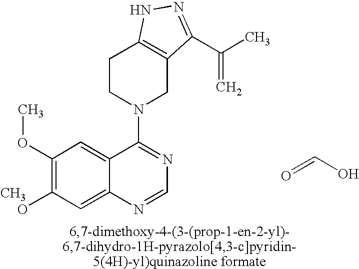

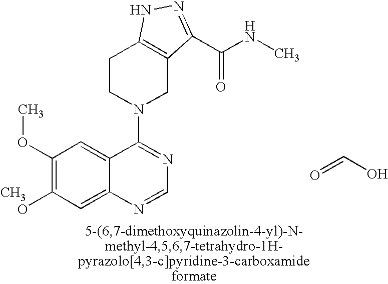

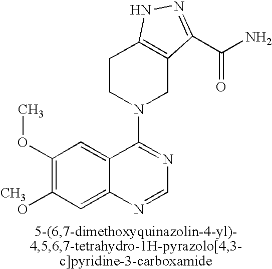

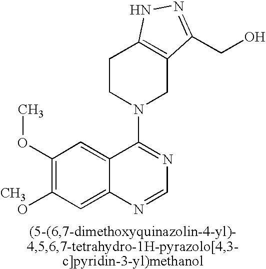

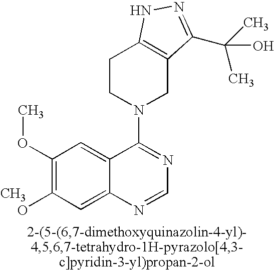

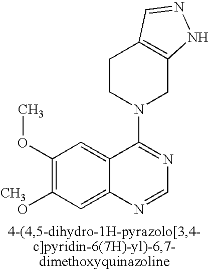

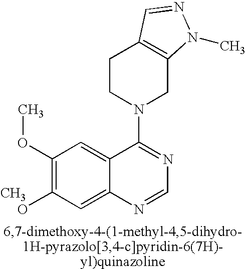

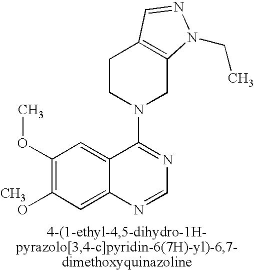

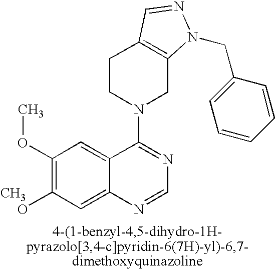

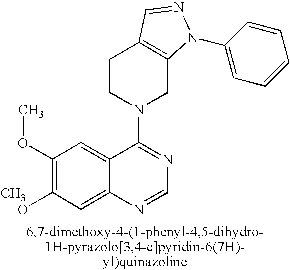

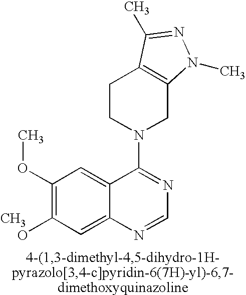

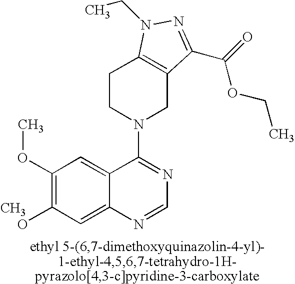

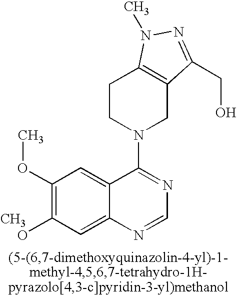

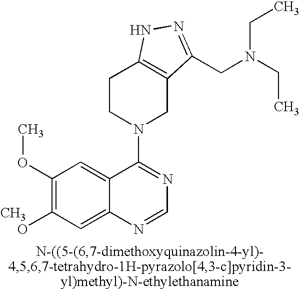

The compounds described herein may be prepared conventionally. Some of the known processes that can be used are described below. Quinazoline compounds disclosed as inhibitors of PDE-10 are also described in published US patent application no. US 2005/0182079, the entire disclosure of which is hereby incorporated by reference.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). 4-chloro-6,7-dimethoxyquinazoline may be obtained from ChemPacific Corp. (Baltimore, Md.), Oakwood Products, Inc. (West Columbia, S.C.) or Fluorochem (Derbyshire, UK). These schemes are merely illustrative of some methods by which the compounds described herein can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., for example, from about 0° C. to about 125° C., such as at about room (or ambient) temperature, e.g., about 20° C.

Compounds of Formula (I) can be prepared as described below. The core heterocyclic entity of each of the drug candidates described is a 6,7-disubstituted quinazoline. These molecules have been prepared by several effective methods (see, e.g., Lednicer, D., Strategies for Organic Drug Synthesis and Design, John Wiley & Sons, Inc. 1998, pp 346-351 and references cited therein). One method (see Scheme 1 below) involves reaction of ortho-aminobenzamides 1 with triethylorthoformate to generate 4-quinazolones 2. Reaction with phosphorous oxychloride generates the starting material 4-chloroquinazoline 3. Some 4-chloroquinazoline starting materials are commercially available, such as 4-chloro-6,7-dimethoxyquinazoline.

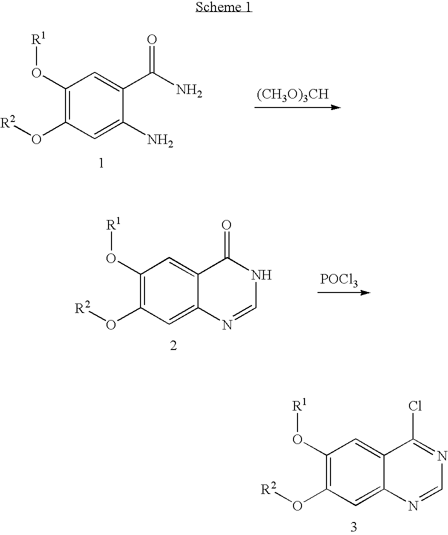

Alternatively, ortho-aminobenzoate esters 4 undergo reaction with formamide to generate quinazolones 2, which are then converted to 4-chloroquinazolines 3 by treatment with phosphorous oxychloride.

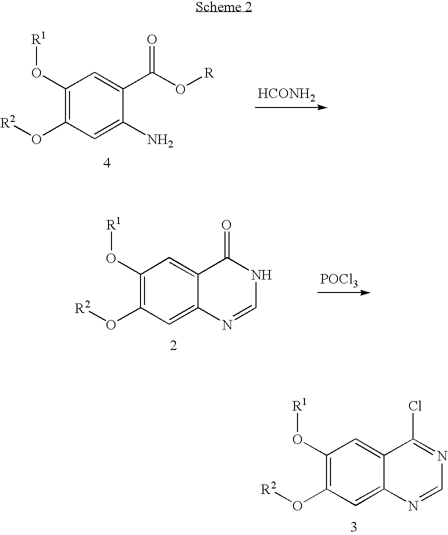

4,5-Disubstituted 2-aminobenzamides 1 and 2-aminobenzoates 2 are either commercially available (e.g., methyl 2-amino-4,5-dimethoxybenzoate) or can be synthesized by methods common to the art. Simple dialkyl ethers, wherein the alkyl groups at the 3,4-positions are the same, can be readily accessed by standard etherification reactions. For example, 6,7-dimethoxy-4-quinazolone can be converted to 6,7-dihydroxy-4-quinazolone
5 by treatment with BBr₃, which in turn can undergo standard etherification type reactions, such as by treatment with an excess of cesium carbonate and an alkyl halide, to provide the dialkylated product. Other bases such as triethylamine, sodium hydride, potassium carbonate, potassium hydride, etc. can be employed in combination with a variety of solvents, including acetone, acetonitrile, DMF, and THF.

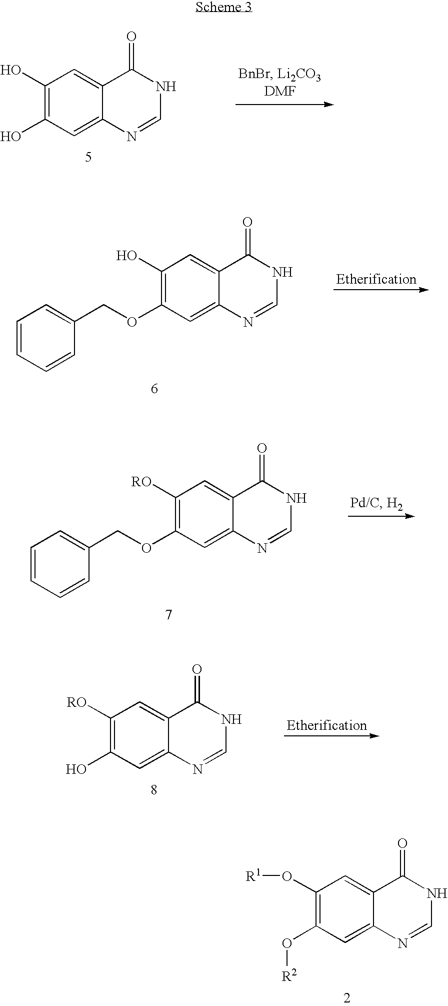

Syntheses of differentially substituted 3,4-dialkyl ethers of 2 can be accomplished via methods known in the art. For example, as shown in Scheme 3 above, 6,7-dihydroxy-4-quinazolone 5 can be utilized as the starting material and selectively protected as its 7-benzyl ether 6 [Greenspan, Paul D. et al., J. Med. Chem., 1999, 42, 164.] by treatment with benzyl bromide and lithium carbonate in DMF solution. Functionalization of the remaining phenol group with the desired alkyl halide to generate the 6-alkoxy-7-benzyloxy-4-quinazolone 7 can be accomplished by any of etherification reactions described above, including Mitsunobu reaction. Removal of the benzyl ether by hydrogenolysis over palladium on carbon in alcoholic solvents such as methanol provides the 7-hydroxy derivative 8, which undergoes a final etherification to yield 3,4-dialkoxyacetophenones 2.

For many of the differentially substituted dialkoxy ethers, other starting materials can prove useful. A large number of substituted 3,4-dialkoxybenzoates are commercially available or are readily synthesized as outlined in Scheme 4 below. Selective benzoylation of catechol 9 with benzyl bromide and lithium carbonate gives 10. An etherification reaction provides 11, and subsequent hydrogenation of the benzyl group and further etherification provides 3,4-dialkoxybenzoates 12. Nitration, followed by nitro group reduction provides precursor compound 4.

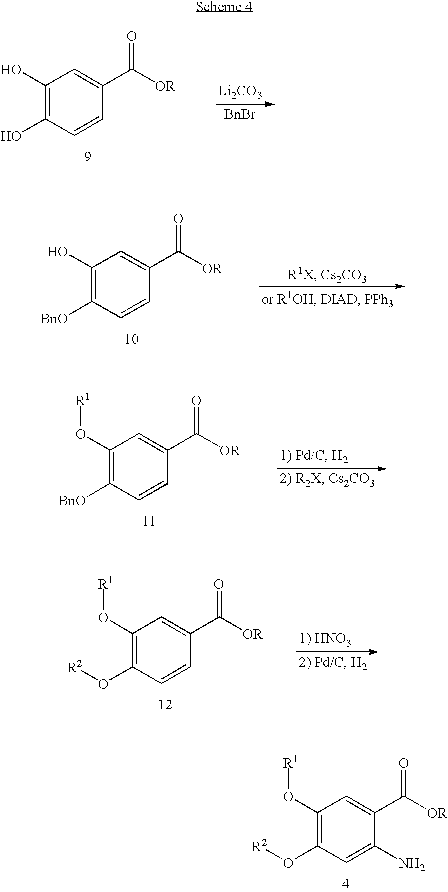

The 4-haloquinazolines (such as 4-chloroquinazoline 3) can be coupled can then be converted to a compound of Formula I or II. Compounds of Formula I where R³ is nitrogen containing group attached to the quinazoline ring via the nitrogen atom such as tetrahydroisoquinolines can be heated directly, either conventionally or in the microwave see [Lowrie, Harman S. J. Med. Chem., 1966, 9, 670.]

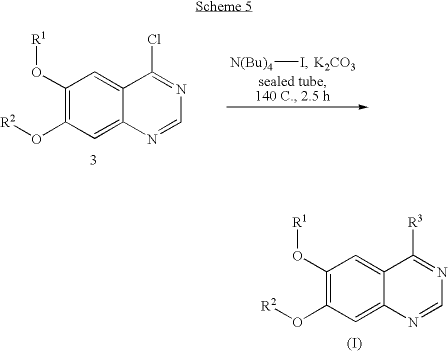

Alternatively, the coupling can be carried out in the presence of palladium. A large variety of conditions are effective in these reactions. Palladium sources include, for example, Pd(PPh₃)₄, Pd₂(dba)₃, Pd(OAc)₂, and others, while solvents such as toluene, DMF, THF, and acetonitrile may be employed. Bases and ligands have also been explored extensively, and may include, for example, NaOtBu, NaHMDS, NaOMe, Cs₂CO₃, and other bases. Ligands which may be employed include, but are not limited to, dppb, XANPHOS, BINAP, tBu₃P, and 2-dicyclohexylphosphino-2′-(N,N-dimethylamino)biphenyl. Optimal reaction conditions vary depending on the nitrogen containing substrate used and also on the haloquinazoline starting material. In the example shown in Scheme 5, Pd₂(dba)₃ may be used as the palladium source, with XANPHOS as the ligand and sodium t-butoxide as the base in toluene solution. To complete the couplings, the reactions are generally heated to between about 50 and 100° C. for about 18 hours. Microwave heating may also be effective in many cases.

Compounds of Formula I where R³ is aryl or heteroaryl can be prepared by reacting 4-haloquinazolines 3 under Suzuki coupling reactions (Scheme 6 below) to yield 4-aryllheteroarylquinazoline compounds of Formula (I).

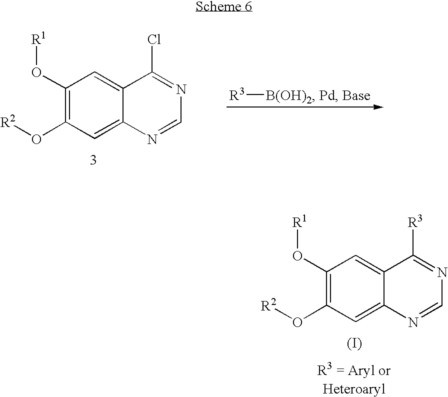

Compound of Formula (II) can be prepared by coupline carbon nucleophiles generated by treatment of an activated alkyl with base to halo-quinazolines 3 under nucleophilic displacement reaction conditions (Scheme 7). Generally, these reactions can be accomplished if one of the substituents (R¹⁶ or R¹⁷) is aromatic or otherwise resonance withdrawing to provide stabilization to the developing anion. A variety of different conditions can be employed. Typically a strong base such as KHMDS, NaNH₂, or LDA is utilized to deprotonate the side chain substrate at temperatures from about −78° C. to about 0° C. The haloquinazoline is then added to the anion as a solution in solvents such as THF, DMF, or benzene, and the reactions are generally warmed to room temperature until complete.

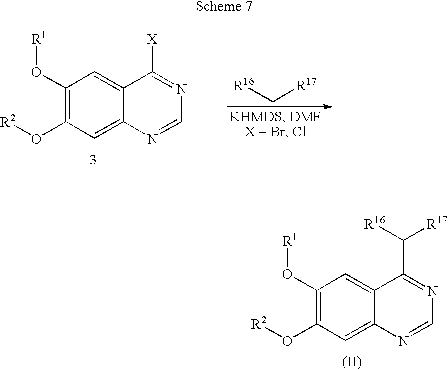

The formation of imidazoline heterocycles 18 requires the generation of a variety of substituted diamines 17 to be synthesized. Thus, resin supported chloroacetamides can be reacted with amines, followed by amide reduction and then cleavage from the resin to provide appropriately substituted diamines 17. A combinatorial reaction approach is effective. [Barry, Clifton E. et al. J. Comb. Chem., 2003, 5, 172.]

Requisite diamines 17 and correspondingly, the cyano-imidazolines 18, can be prepared from nitro alcohols 15 as outlined in Scheme 8 [Senkus, Murray et al. J. Am. Chem. Soc. 1946, 68, 10]

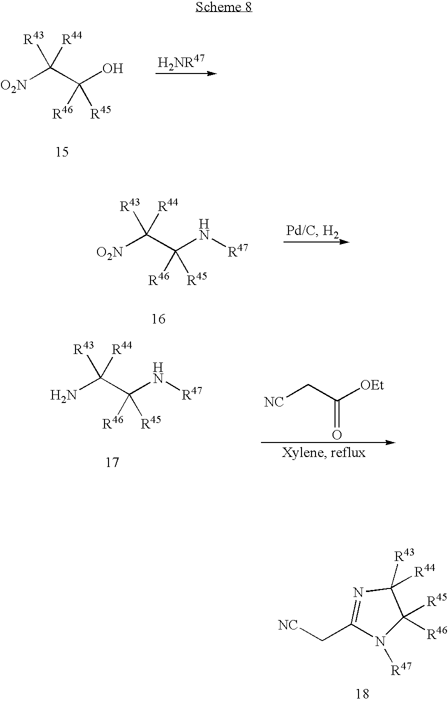

Thus, heating substituted nitro ethanols 15 with primary amines (condensation reaction) provides nitro ethylamines 16. Reduction of the nitro group to the corresponding amine by hydrogenation over palladium on carbon or with iron powder provides precursor diamines 17. Condensation with ethyl cyanoacetate provides the desired cyanoimidazolines 18. [Riebsomer, J. L. et al., J. Org. Chem. 1950, 15, 909.]

An alternative approach to the desired cyanoimidazolines 18 involves cyclization of diamines 17 with cyano-imidate 19. [Meyers, A. I. et al. Tetrahedron, 2002, 58, 207.] Treatment of the imidate 19 with amino alcohols or amino thiols 20 provides oxazoline and thizoline heterocycles 21 (Scheme 9).

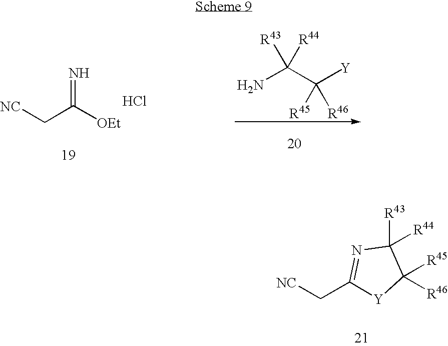

Various carboxylate derivatives can be obtained from the cyano-heterocycle side chains appended to quinazoline 14. Reduction of the nitrile provides amines, which can be further manipulated; while hydrolysis of the nitrile provides carboxamides and carboxylic acids.

Several methods are available for the synthesis of variously substituted tetrahydroisoquinoline (THIQ) compounds. For example, commercially available THIQ 22 can be protected as the 1-amido analog 23 by reaction with acetic anhydride or acetyl chloride and base (Scheme 10). Cleavage of the methoxy group with BBr₃ provides phenolic intermediate 24, which undergoes alkylation reactions with various alkyl halides such as methoxyethyl chloride to generate 1-amido analogs 25, which can be hydrolyzed under basic conditions to yield target THIQ compounds 26.

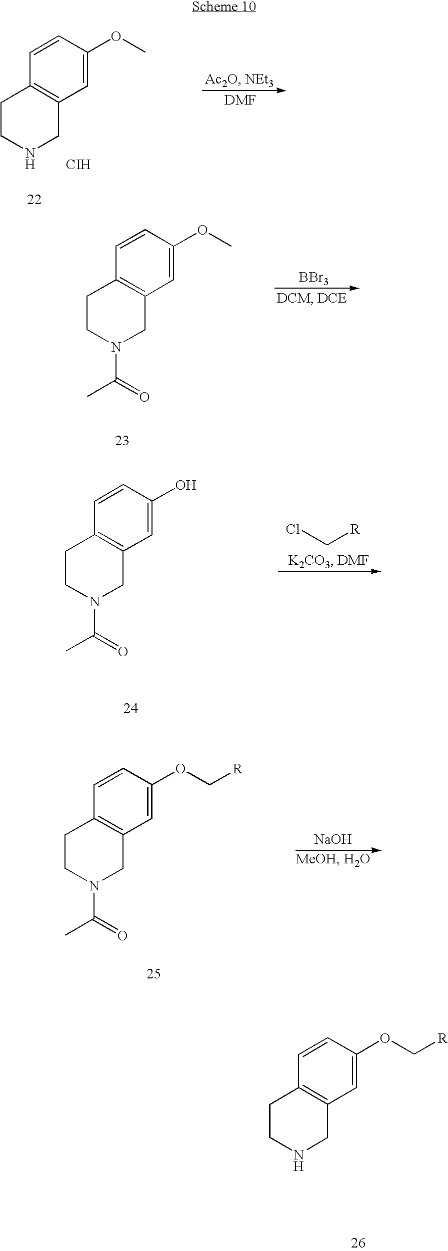

Alternatively, THIQ compounds can be synthesized from phenethylamines 27 by reaction with ethyl chloroformate to generate carbamates of the type 28. Acid promoted cyclization yields dihydroquinolones 29 which are reduced to target THIQ compounds by reaction with lithium aluminum hydride (LAH) (Scheme 11).

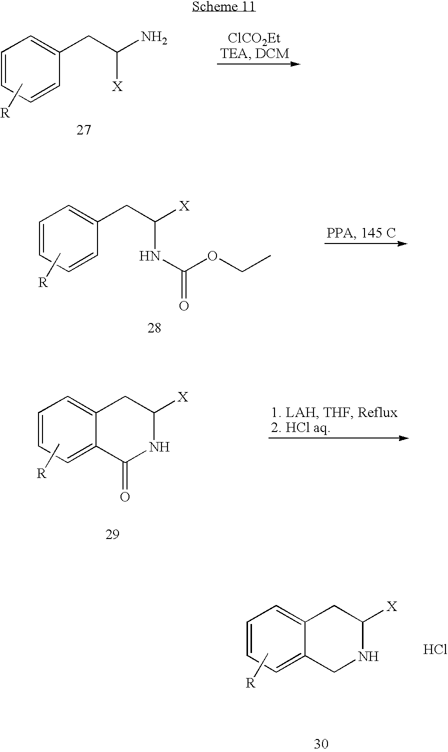

The THIQ compounds can be further functionalized by generating phenol 32 from the corresponding methoxy derivative 31 by reaction with BBr₃, followed by alkylation-type reactions. Thus, as exemplified in Scheme 12, dihydroisoquinoline 32 undergoes reaction with alkyl halides, for example 1-chloro-2-ethoxyethane, in the presence of a base (such as K₂CO₃) and a phase transfer catalyst to provide alkyloxy intermediate 33. Subsequent reduction of the amide with borane provides target 26.

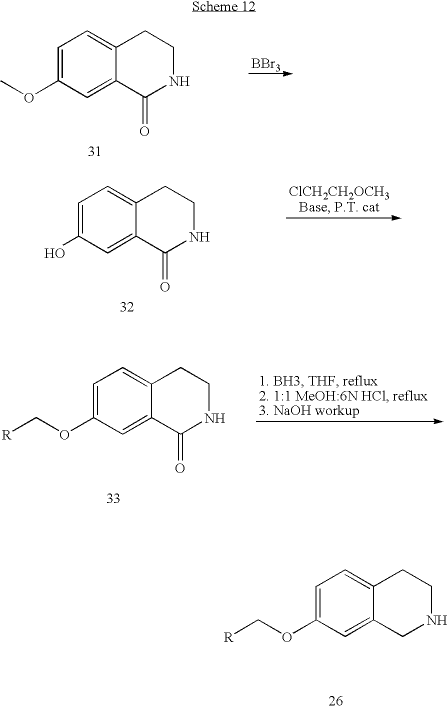

Further, the phenol derivatives 34 can undergo arylation and heteroarylation reactions (Scheme 13) with appropriately substituted boronic acids to yield dihydroisoquinilones of the type 35. Reduction with LAH produces THIQ targets 36.

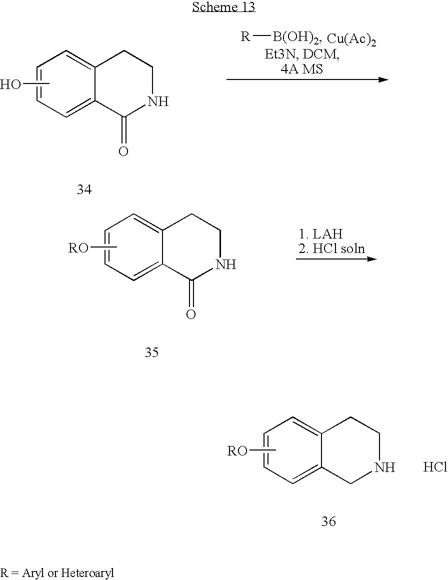

Furthermore, phenols 34 can be converted to the corresponding triflates which may undergo reaction with aryl and heteroaryl boronic acids to yield aryl and heteroaryl substituted tetrahydroisoquinolines 39 after treatment with LAH (scheme 14). In addition, it is possible to displace the triflate with a variety of amines under Buchwald conditions.

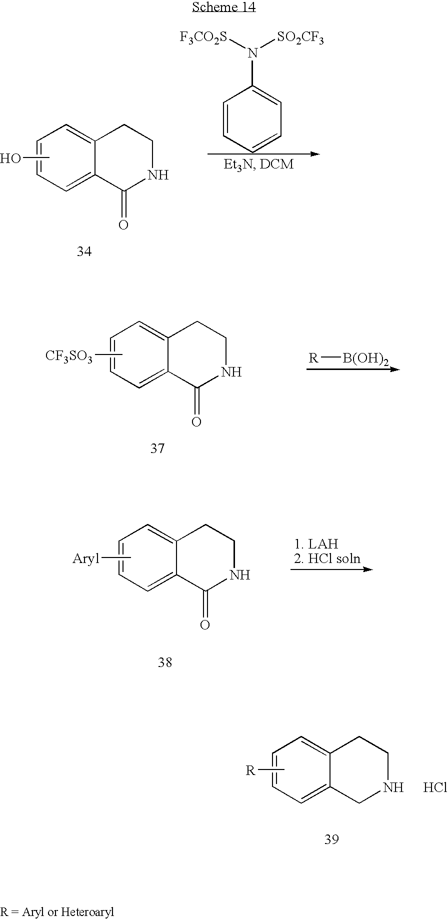

Nitration of dihydroisoquinolones of the type 40 by reaction with nitric acid and sulfuric acid produces 7-nitrodihydroisoquinolones 41 (scheme 15). Borane reduction to 7-nitrotetrahydroisoquinoline 42 followed by acetylation with trifluoroacetic anhydride provides protected nitro analog 43. Reductive hydrogenation over palladium on carbon and subsequent acetylation with acetic anhydride generates acetamide 44. Trifluoroacetamide hydrolysis by reaction with potassium carbonate in methanol produces tetrahydroisoquinoline 45.

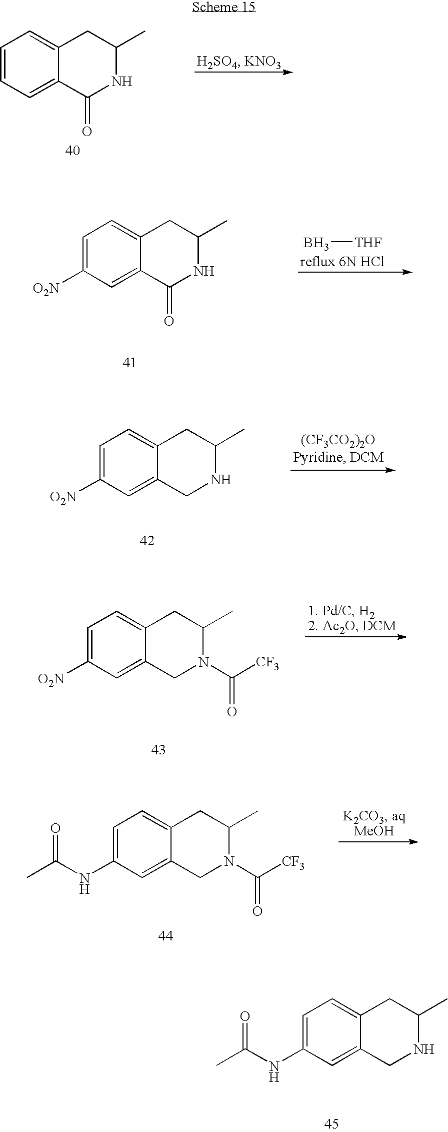

Aminosulfonyl substituted tetrahydroquinolines 49 can be synthesized in 3 steps from N-acetyltetrahydroquinoline 46 (scheme 16). Thus, treatment of 46 with chlorosulfonic acid provides 6-chlorosulfonyl derivative 47. Reaction with an amine, for example dimethylamine, and subsequent acid induced hydrolysis of the acetamide provides target 49.

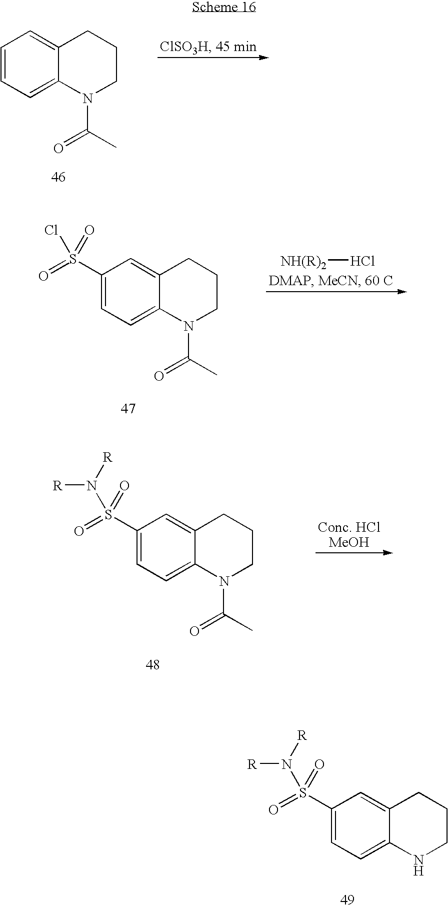

Dihydroquinolones 52 and tetrahydroquinolines such as 53 can be prepared as described in scheme 17. Thus, diazotization and then reaction with sulfur dioxide and cuprous chloride provides sulfonyl chloride derivative 51. Reaction with amines, such as dimethylamine, provides sulfonamide dihydroquinolones 52, which may be readily reduced by reaction with borane in THF to generate the corresponding tetrahydroquinolines 53.

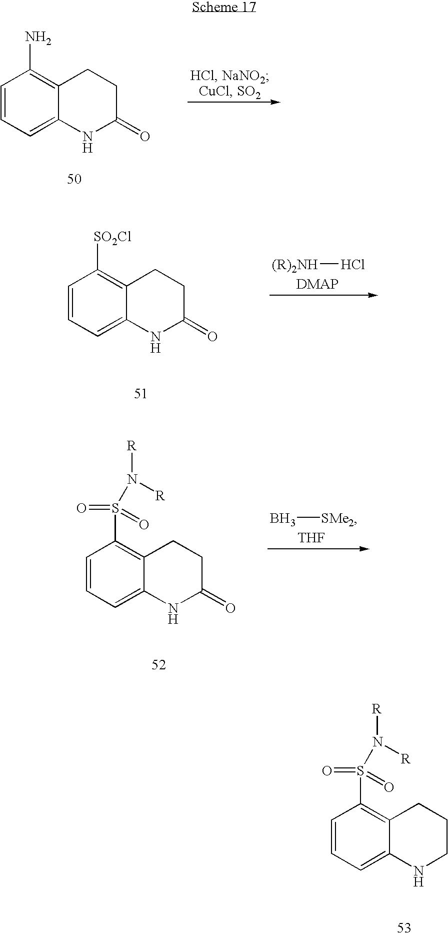

Amino-dihydroquinoline 50 undergoes reaction with alkylsulfonyl halides (such as methanesulfonyl chloride) to yield N,N-dialkylsulfonylamino derivatives (e.g., N,N-dimethanesulfonylamino derivative 54) (scheme 18). Reduction of the dihydroquinoline to the tetrahydroquinoline with borane and subsequent treatment with lithium hydroxide yields 5-alkylsulfonamido-tetrahydroquinolines 56 (e.g., 5-methylsulfonamido-tetrahydroquinoline).

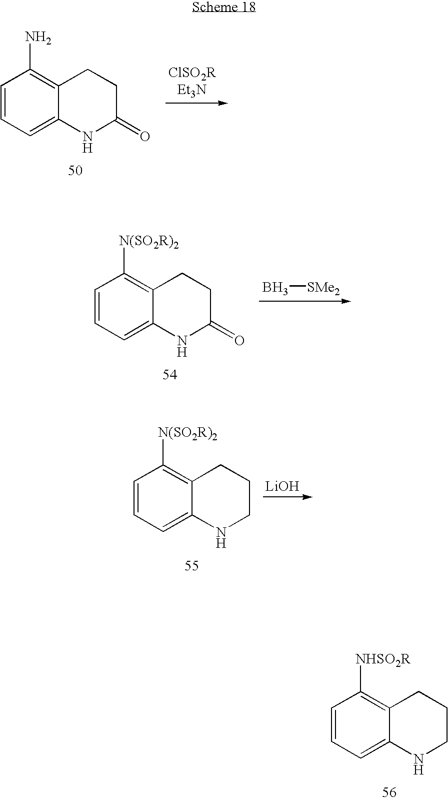

Aminosulfonyl indoline compounds (scheme 19) can be prepared in a similar manner as described in scheme 17. Thus, N-acetyl 5-chlorosulfonylindolines 57 undergo reactions with amines to generate aminosulfonylindolines 59 after N-acetyl hydrolysis of 58 using sodium hydroxide.

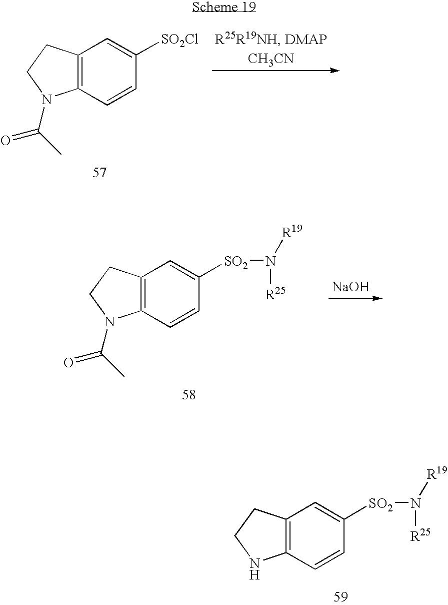

Substituted pyrrazolotetrahydropyridine compounds 66 and 67 can be prepared as described in scheme 20. Thus, BOC-protected ethyl-4,5,6,7-tetrahydro-1H-pyrazolo[4,3-c]pyridine-3-carboxylate 63 can be treated with trifluoracetic acid to generate analog 67, or can be hydrolyzed with a base, such as sodium hydroxide, to yield acid 64. Thus, BOC-protected ethyl-4,5,6,7-tetrahydro-11H-pyrazolo[4,3-c]pyridine-3-carboxylic acid
64 undergoes reactions with amines to generate 66 after deprotection of 65 under acidic conditions.

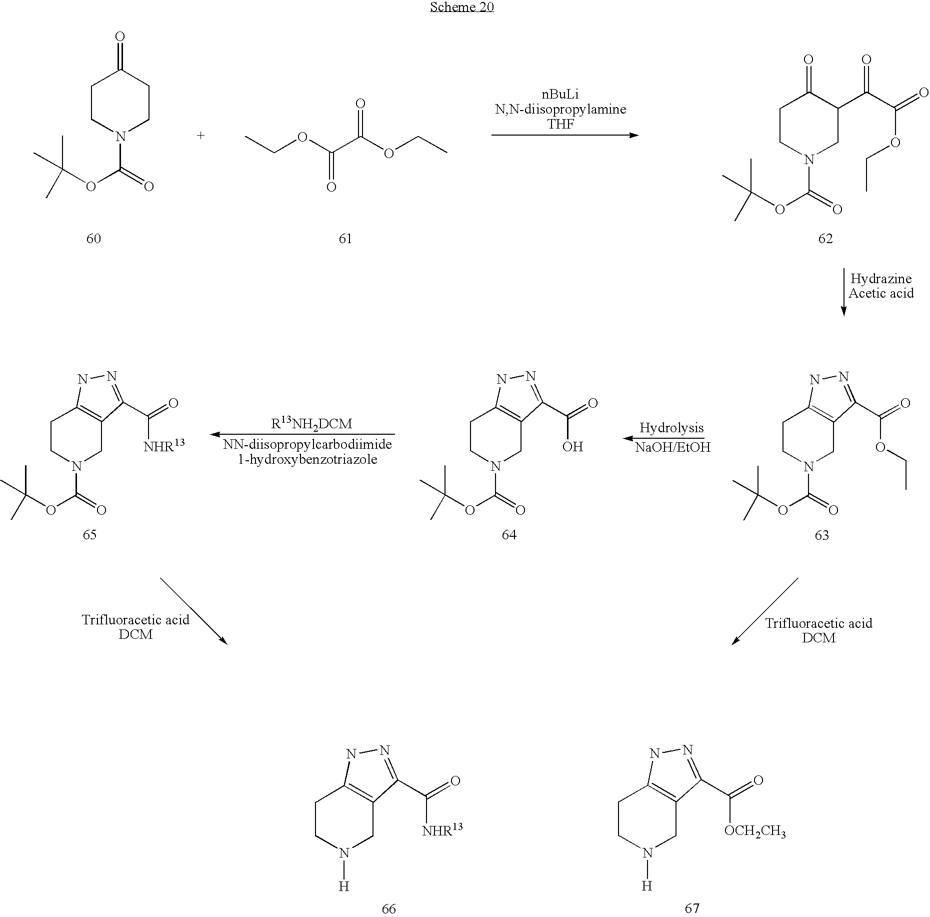

The chemical entities described herein inhibit PDE10 enzyme activity and hence raise the levels of cAMP or cGMP within cells that express PDE10. Accordingly, inhibition of PDE 0 enzyme activity would be useful in the treatment of diseases caused by deficient amounts of cAMP or cGMP in cells. PDE10 inhibitors would also be of benefit in cases wherein raising the amount of cAMP or cGMP above normal levels results in a therapeutic effect. Inhibitors of PDE10 may be used to treat disorders of the peripheral and central nervous system, cardiovascular diseases, cancer, gastro-enterological diseases, endocrinological diseases and urological diseases.

Indications that may be treated with PDE10 inhibitors, either alone or in combination with other drugs, include, but are not limited to, those diseases thought to be mediated in part by the basal ganglia, prefrontal cortex and hippocampus. These indications include psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease. For example, the PDE10 inhibitors described herein can be used in combination with other pharmaceutical agents such as other agents used in the treatment of psychoses, such as schizophrenia and bipolar disorder, obsessive-compulsive disorder, Parkinson's disease, cognitive impairment and/or memory loss, e.g., nicotinic α-7 agonists, PDE4 inhibitors, other PDE10 inhibitors, calcium channel blockers, muscarinic m1 and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigmine, and galanthanamine). In such combinations, each active ingredient can be administered either in accordance with their usual dosage range or a dose below their usual dosage range.

Psychoses are disorders that affect an individual's perception of reality. Psychoses are characterized by delusions and hallucinations. The chemical entities described herein may be useful in treating patients suffering from all forms of psychoses, including, but not limited to, schizophrenia, late-onset schizophrenia, schizoaffective disorders, prodromal schizophrenia, and bipolar disorders. Treatment may be for the positive symptoms of schizophrenia as well as for the cognitive deficits and negative symptoms. Other indications for PDE10 inhibitors include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality may also be treated with PDE10 inhibitors.

Obsessive-compulsive disorder (OCD) has been linked to deficits in the frontal-striatal neuronal pathways. (Saxena S. et al., Br. J. Psychiatry Suppl., 1998; (35):26-37.) Neurons in these pathways project to striatal neurons that express PDE10. PDE10 inhibitors cause cAMP to be elevated in these neurons; elevations in cAMP result in an increase in CREB phosphorylation and thereby improve the functional state of these neurons. The chemical entities described herein may be useful for the indication of OCD. OCD may result, in some cases, from streptococcal infections that cause autoimmune reactions in the basal ganglia (Giedd J N et al., Am J Psychiatry., 2000 February; 157(2):281-3). Because PDE10 inhibitors may serve a neuroprotective role, administration of PDE10 inhibitors may prevent the damage to the basal ganglia after repeated streptococcal infections and thereby prevent the development of OCD.

In the brain, the level of cAMP or cGMP within neurons is believed to be related to the quality of memory, such as long term memory. Without wishing to be bound to any particular mechanism, it is proposed that since PDE10 degrades cAMP or cGMP, the level of this enzyme affects memory in animals, for example, in humans. For example, a compound that inhibits cAMP phosphodiesterase (PDE) can thereby increase intracellular levels of cAMP, which in turn activate a protein kinase that phosphorylates a transcription factor (cAMP response binding protein), which transcription factor then binds to a DNA promoter sequence to activate genes that are important in long term memory. The more active such genes are, the better is long-term memory. Thus, by inhibiting a phosphodiesterase, long term memory can be enhanced.

Dementias are diseases that include memory loss and additional intellectual impairment separate from memory. The chemical entities described herein may be useful for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeldt-Jakob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

Methods described herein include, but are not limited to, methods of enhancing cognition in a patient in whom such enhancement is desired, methods of treating a patient suffering from cognition impairment or decline, methods of treating a patient having a disease involving decreased cAMP and/or cGMP levels, methods of inhibiting PDE10 enzyme activity in a patient, methods of treating a patient suffering psychoses, in particular schizophrenia or bipolar disorder, methods of treating a patient suffering from obsessive-compulsive disorder, and methods of treating a patient suffering from Parkinson's disease.

The condition of memory impairment is manifested by impairment of the ability to learn new information and/or the inability to recall previously learned information. Provided are methods for dealing with memory loss separate from dementia, including mild cognitive impairment (MCI) and age-related cognitive decline. Also provided are methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeldt-Jakob disease, HIV, cardiovascular disease, and head trauma as well as age-related cognitive decline. The chemical entities described herein may be useful in the treatment of memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeldt-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases.

The chemical entities described herein may also be suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include dentatorubral-pallidoluysian atrophy, DRPLA (atrophin-1); spinocerebellar ataxia type-1 (ataxin-1); spinocerebellar ataxia type-2 (ataxin-2); spinocerebellar ataxia type-3 also called Machado-Joseph disease, MJD (ataxin-3); spinocerebellar ataxia type-6 (alpha 1a-voltage dependent calcium channel); spinocerebellar ataxia type-7 (ataxin-7); and spinal and bulbar muscular atrophy, SBMA, also know as Kennedy disease (androgen receptor).

The basal ganglia are important for regulating the function of motor neurons; disorders of the basal ganglia result in movement disorders. Most prominent among the movement disorders related to basal ganglia function is Parkinson's disease (Obeso J A et al., Neurology., 2004 Jan. 13; 62(1 Suppl 1):S17-30). Other movement disorders related to dysfunction of the basal ganglia include tardive dyskinesia, progressive supranuclear palsy and cerebral palsy, corticobasal degeneration, multiple system atrophy, Wilson disease, and dystonia, tics, and chorea. The chemical entities described herein may be used to treat movement disorders related to dysfunction of basal ganglia neurons.

PDE10 inhibitors can be used to raise cAMP or cGMP levels and prevent neurons from undergoing apoptosis. PDE10 inhibitors may be anti-inflammatory by raising cAMP in glial cells. The combination of anti-apoptotic and anti-inflammatory properties, as well as positive effects on synaptic plasticity and neurogenesis, make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Autoimmune diseases or infectious diseases that affect the basal ganglia may result in disorders of the basal ganglia including ADHD, OCD, tics, Tourette's disease, and Sydenham chorea. In addition, any insult to the brain can potentially damage the basal ganglia including strokes, metabolic abnormalities, liver disease, multiple sclerosis, infections, tumors, drug overdoses or side effects, and head trauma. Accordingly, the chemical entities described herein may be used to stop disease progression or restore damaged circuits in the brain by a combination of effects including increased synaptic plasticity, neurogenesis, anti-inflammatory effects, nerve cell regeneration and decreased apoptosis

The growth of some cancer cells is inhibited by cAMP and cGMP. Upon transformation, cells may become cancerous by expressing PDE10 and reducing the amount of cAMP or cGMP within cells. In these types of cancer cells, inhibition of PDE10 activity will inhibit cell growth by raising cAMP. In some cases, PDE10 may be expressed in the transformed, cancerous cell but not in the parent cell line. In transformed renal carcinoma cells, PDE10 is expressed and PDE10 inhibitors reduce the growth rate of the cells in culture. Similarly, breast cancer cells are inhibited by administration of PDE10 inhibitors. Many other types of cancer cells may also be sensitive to growth arrest by inhibition of PDE 10. Therefore, chemical entities described herein may be used to stop the growth of cancer cells that express PDE 10.

The chemical entities described herein may also be suitable for use in the treatment of diabetes and related disorders such as obesity, by focusing on regulation of the cAMP signaling system. By inhibiting PDE-10A activity, intracellular levels of cAMP are increased, thereby increasing the release of insulin-containing secretory granules and, therefore, increasing insulin secretion. See, for example, WO 2005/012485, which is hereby incorporated by reference in its entirety. The compounds of Formula (I) can also be used to treat the diseases disclosed in US Patent application publication No. 2006/019975, the disclosure of which is incorporated herein by reference in its entirety.

Also provided is a method of treating diabetes and related disorders comprising administering to a patient, such as a mammal, such as a human, a therapeutically effective amount of at least one chemical entity described herein. In accordance with a further embodiment, there is provided a method of treating type 1 diabetes, type 2 diabetes, Syndrome X, impaired glucose tolerance, impaired fasting glucose, gestational diabetes, maturity-onset diabetes of the young (MODY), latent autoimmune diabetes adult (LADA), associated diabetic dyslipidemia, hyperglycemia, hyperinsulinemia, dyslipidemia, hypertriglyceridemia, and insulin resistance, comprising administering to a patient, such as a mammal, such as a human, a therapeutically effective amount of at least one chemical entity described herein.

A subject or patient in whom administration of the therapeutic compound is an effective therapeutic regimen for a disease or disorder is, in some embodiments, a human, but can be any animal, including a laboratory animal in the context of a clinical trial or screening or activity experiment. Thus, as can be readily appreciated by one of ordinary skill in the art, the chemical entities described herein may be administered to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

Assays for determining PDE10 inhibiting activity, selectivity of PDE10 inhibiting activity, and selectivity of inhibiting PDE10 isoenzymes are known within the art. See, e.g., U.S. Published Application No. 2004/0162293. The PDE10 inhibitory activities of chemical entities described herein may be tested using the in vitro assay described below.

In general, the chemical entities described herein may be administered in a therapeutically effective amount by any of the accepted modes of administration for agents that serve similar utilities. The actual amount of the at least one chemical entity described herein, i.e., the active ingredient(s), will depend upon numerous factors such as the severity of the disease to be treated, the age and relative health of the subject, the potency of the compound used, the route and form of administration, the efficacy, toxicology profile, pharmacokinetic profile of the compound, and the presence of any deleterious side-effects, among other considerations.

Therapeutically effective amounts of compounds of formula (I) may range from approximately 0.001-100 mg/kg/day, for example, 0.01-100 mg/kg/day, such as 0.1-70 mg/kg/day, and in some embodiments, 0.5-10 mg/kg/day. In other embodiments, the therapeutically effective amount may range from 0.005-15 mg per kilogram body weight of the recipient per day; for example, about 0.05-1 mg/kg/day. Thus, for administration to a 70 kg person, the dosage range would be about 3.5 mg to 70 mg per day. For intravenous administration, the compounds can be administered, in single or multiple dosages, at a dosage level of, for example, 0.001-50 mg/kg/day, for example, 0.001-10 mg/kg/day, such as, 0.01-1 mg/kg/day.

Unit dosage forms for oral administration can contain generally 0.01-1000 mg of active compound, for example, 0.1-50 mg of active compound. Unit dosage forms for intravenous administration can contain, for example, 0.1-10 mg of active compound.

In general, the chemical entities described herein may be administered as pharmaceutical compositions by any one of the following routes: oral, systemic (e.g., transdermal, intranasal or by suppository), or parenteral (e.g., intramuscular, intravenous, subcutaneous, intrasternal and by infusion) administration, by inhalation and by ocular administration. In some embodiments, the manner of administration is oral using a convenient daily dosage regimen which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules may be used) and the bioavailability of the drug substance. Recently, pharmaceutical formulations have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Various solid oral dosage forms can be used for administering chemical entities described herein including such solid forms as tablets, gelcaps, capsules, caplets, granules, lozenges and bulk powders. The chemical entities described herein may be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels may be used to administer the chemical entities described herein.

Various liquid oral dosage forms can also be used for administering chemical entities described herein, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the chemical entities described herein. The chemical entities described herein may be injected, for example, intravenously, in the form of an isotonic sterile solution. Other preparations are also possible.

Suppositories for rectal administration of the chemical entities described herein may be prepared by mixing the compound with a suitable excipient such as cocoa butter, salicylates and polyethylene glycols. Formulations for vaginal administration may be in the form of a pessary, tampon, cream, gel, paste, foam, or spray formula containing, in addition to the active ingredient, such suitable carriers as are known in the art.

For topical administration, the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the chemical entities described herein can be administered by inhalation in the form of a powder (e.g., micronized) or in the form of atomized solutions or suspensions. The aerosol formulation can be placed into acceptable propellant.

The compositions are comprised of in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. In some embodiments, liquid carriers, particularly for injectable solutions, are chosen from water, saline, aqueous dextrose, and glycols.

Compressed gases may be used to disperse chemical entities described herein in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The level of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. In some embodiments, the compound is present at a level of about 1-80 wt %.

The compounds can be administered as the sole active agent or in combination with other pharmaceutical agents such as other agents used in the treatment of psychoses, such as schizophrenia and bipolar disorder, obsessive-compulsive disorder, Parkinson's disease, Alzheimer's disease, cognitive impairment and/or memory loss, e.g., nicotinic α-7 agonists, PDE4 inhibitors, other PDE10 inhibitors, calcium channel blockers, muscarinic m1 and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigmine, and galanthanamine). In such combinations, each active ingredient can be administered either in accordance with their usual dosage range or a dose below their usual dosage range and can be administered prior to, concurrently with, or following administration of the additional pharmaceutical agent or agents.

Drugs suitable for use in combination with the chemical entities described herein include, but are not limited to, other suitable schizophrenia drugs such as Clozaril, Zyprexa, Risperidone, and Seroquel; bipolar disorder drugs such as Lithium, Zyprexa, and Depakote, Parkinson's disease drugs such as Levodopa, Parlodel, Permax, Mirapex, Tasmar, Contan, Kemadin, Artane, and Cogentin; agents used in the treatment of Alzheimer's disease such as, but not limited to, Reminyl, Cognex, Aricept, Exelon, Akatinol, Neotropin, Eldepryl, Estrogen and Cliquinol; agents used in the treatment of dementia such as, but not limited to, Thioridazine, Haloperidol, Risperidone, Cognex, Aricept, and Exelon; agents used in the treatment of epilepsy such as, but not limited to, Dilantin, Luminol, Tegretol, Depakote, Depakene, Zarontin, Neurontin, Barbita, Solfeton, and Felbatol; agents used in the treatment of multiple sclerosis such as, but not limited to, Detrol, Ditropan XL, OxyContin, Betaseron, Avonex, Azothioprine, Methotrexate, and Copaxone; agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone; agents useful in the treatment of diabetes, including, but not limited to, PPAR ligands (e.g. agonists, antagonists, such as Rosiglitazone, Troglitazone and Pioglitazone), insulin secretagogues (for example, sulfonylurea drugs (such as Glyburide, Glimepiride, Chlorpropamide, Tolbutamide, and Glipizide) and non-sulfonyl secretagogues), α-glucosidase inhibitors (such as Acarbose, Miglitol, and Voglibose), insulin sensitizers (such as the PPAR-γ agonists, e.g., the glitazones; biguanides, PTP-1B inhibitors, DPP-IV inhibitors and 11beta-HSD inhibitors), hepatic glucose output lowering compounds (such as glucagon antagonists and metaformin, such as Glucophage and Glucophage XR), insulin and insulin derivatives (both long and short acting forms and formulations of insulin), and anti-obesity drugs (such as β-3 agonists, CB-1 agonists, neuropeptide Y5 inhibitors, Ciliary Neurotrophic Factor and derivatives (e.g., Axokine), appetite suppressants (e.g., Sibutramine), and lipase inhibitors (e.g., Orlistat)).

In carrying out the procedures described herein, it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

### EXAMPLES

The following preparations and examples are given to enable those skilled in the art to more clearly understand and to practice the present invention. They should not be considered as limiting the scope of the invention, but merely as being illustrative and representative thereof. All spectra were recorded at 300 MHz on a Bruker Instruments NMR unless otherwise stated. Coupling constants (j) are in Hertz (Hz) and peaks are listed relative to TMS (δ 0.00 ppm). Microwave reactions were performed using a Personal Chemistry Optimizer™ microwave reactor in 10 mL Personal Chemistry microwave reactor vials. All reactions were performed at 200° C. for 600 s with the fixed hold time ON unless otherwise stated. Sulfonic acid ion exchange resins (SCX) were purchased from Varian Technologies. Analytical HPLC was performed on 4.6 mm×100 mm Waters Sunfire RP C18 5 μm column using (i) a gradient of 20/80 to 80/20 acetonitrile (0.1% formic acid)/water (0.1% formic acid) over 6 min (Method A), (ii) a gradient of 20/80 to 80/20 acetonitrile (0.1% formic acid)/water (0.1% formic acid) over 8 min (Method B), (iii) a gradient of 40/60 to 80/20 acetonitrile (0.1% formic acid)/water (0.1% formic acid) over 6 min (Method C), (iv) a gradient of 40/60 to 80/420 acetonitrile (0.1% formic acid)/water (0.1% formic acid) over 8 min (Method D), (v) an isocratic eluent of 80/20 acetonitrile (0.1% formic acid)/water (0.1% formic acid) over 8 minutes (Method E), (vi) a gradient of 10/90 to 90/10 acetonitrile (0.1% formic acid)/water (0.1% formic acid) over 6 min (Method F), (vii) a gradient of 10/90 to 60/40 acetonitrile (0.1% formic acid)/water (0.1% formic acid) over 6 min (Method G), (viii) a gradient of 10-60% acetonitrile/water (0.1% formic acid) over 6 min (Method H), (ix) a gradient of 10/90 to 60/40 acetonitrile (0.1% formic acid)/water (0.1% formic acid) over 8 min (Method I), or (x) a gradient of 5/95 to 60/40 acetonitrile (0.1% formic acid)/water (0.1% formic acid) over 8 min (Method J). Preparative HPLC was performed on 30 mm×100 mm Xtera Prep RP₁₈ 5μ columns using an 8 min gradient of 95/5 to 20/80 water (0.1% formic acid)/acetonitrile (0.1% formic acid).

### Synthetic Examples

### Example 1

### (4,5-Dihydro-1-isopropyl-1H-imidazol-2-yl)acetonitrile

Ethyl 2-cyanoethanimidoate hydrochloride (500 mg, 3.3650 mmol) was dissolved in dry methylene chloride (5 mL) under an atmosphere of argon. N-isopropylethylenediamine (0.416 ml, 3.36 mmol) was added and the reaction was stirred for 18 hours. Saturated NaHCO₃ (20 mL) was then added and the mixture was extracted with ethyl acetate (2×10 mL), washed with a saturated solution of NH₄Cl (2×10 mL), dried (MgSO₄), filtered, and concentrated to provide 313 mg (62%) of (4,5-dihydro-1-isopropyl-1H-imidazol-2-yl)acetonitrile as a light brown solid. MS [M+H]=152, ¹H NMR (CDCl₃) δ (ppm) 5.02 (br s, 1H), 3.52 (m, 1H), 3.35 (m, 4H), 2.95 (s, 1H), 1.15 (s, 3H), 1.09 (s, 3H).

### Example 2

### (1-Benzyl-4,5-dihydro-1H-imidazol-2-yl)acetonitrile

Cyanoacetic acid, ethyl ester (1.42 mL, 0.0133 mol) and N-benzylethylenediamine (1.00 g, 6.66 mmol) were dissolved in 1,2-dimethylbenzene (50 mL). The reaction mixture was heated to reflux for 18 h with a dean-stark trap affixed. Upon cooling to room temperature, the entire mixture was loaded onto a 10 g SCX column, washed with MeOH (1 volume), eluted with NH₃ in MeOH, and then concentrated to provide the crude product. Purification by rotary chromatography, using a gradient elution from 100% CHCl₃ to 10% MeOH in CHCl₃ provided 279 mg (21%) of (1-benzyl-4,5-dihydro-1H-imidazol-2-yl)acetonitrile as an orange solid. ¹H NMR (CDCl₃δ (ppm) 7.30(m, 5H), 4.92 (br s, 1H), 4.21 (s, 2H), 3.53(m, 2H), 3.37 (m, 2H), 3.18 (s, 1H).

The following compound was prepared in a similar fashion with different starting materials: (See also J. Org. Chem., 15, pp. 909, 1950).
1-(Isopropyl-4,4-dimethyl-4,5-dihydro-1H-imidazol-2-yl)acetonitrile MS [M+H]=180

### Example 3a

### 5-tert-butyl-3-ethyl-1,4,6,7-tetrahydro-5H-pyrazolo[4,3-c]pyridine-3,5-dicarboxylate

n-Butyllithium in pentane (2.0 M, 8.5 mL) was added to a solution of N,N-diisopropylamine (2.4 mL) in tetrahydrofuran at 0° C. The reaction was stirred for 30 minutes at 0° C., then cooled to −78° C. and a solution of 1-BOC-4-piperidone (3.20 g, 0.0161 mol) in tetrahydrofuran (20.0 mL) was added slowly. The reaction mixture was stirred at this temperature for 0.5 h, followed by the addition of a solution of diethyl oxalate (2.48 g, 0.0170 mol) in tetrahydrofuran (10.0 mL). The resulting mixture was allowed to warm to room temperature overnight and then water (200 mL) was added and the aqueous phase was neutralized with 1 N HCl and extracted with ethyl acetate. The organic layer was washed with brine, dried over magnesium sulfate, and filtered. The filtrate was concentrated to yield 3.2 g (66.6%) of tert-butyl-3-[ethoxy(oxo)acetyl]-4-oxopiperidine-1-carboxylate as a yellow oil.

Hydrazine (1.00 mL, 0.0319 mol) was added dropwise (with heat evolution) to a mixture of tert-butyl-3-[ethoxy(oxo)acetyl]-4-oxopiperidine-1-carboxylate (4.00 g, 0.0134 mol) and acetic acid (8.00 mL). The mixture was stirred for 16 hours, poured into ice cold saturated aqueous sodium bicarbonate and the mixture was partitioned between water (50 mL) and ethyl acetate (50 mL). The layers were separated and the organic layer was washed with brine (25 mL), dried (magnesium sulfate), and concentrated in vacuo to afford 3.2 g (81.1%) of 5-tert-butyl-3-ethyl 1,4,6,7-tetrahydro-5H-pyrazolo[4,3-c]pyridine-3,5-dicarboxylate. [M+H]=296, LC/MS (EI) t_{R} 6.52 min (Method B).

### Example 3b

### Ethyl-4,5,6,7-tetrahydro-1H-pyrazolo[4,3-c]pyridine-3-carboxylate

Trifluoroacetic acid (4.1 mL, 0.053 mol) was added to 5-tert-butyl 3-ethyl-1,4,6,7-tetrahydro-5H-pyrazolo[4,3-c]pyridine-3,5-dicarboxylate (0.750 g, 0.00254 mol) and the resulting mixture was stirred for 2 hours at room temperature, then concentrated in vacuo. The residue was dissolved in 3N HCl (25 mL) and washed with ethyl acetate (2×25 mL). The aqueous layer was then neutralized with sodium carbonate, extracted with warm ethyl acetate (3×50 mL), and filtered warm through magnesium sulfate. Concentration in vacuo afforded 39 mg (78.7%) of ethyl-4,5,6,7-tetrahydro-1H-pyrazolo[4,3-c]pyridine-3-carboxylate as a tan solid. ¹H NMR (MeOD) δ (ppm) 4.33 (q, J=7.2, 2H), 3.97 (s, 2H), 3.05 (m, 2H), 2.73 (m, 2H), 1.36 (t, J=7.2, 3H).

### Example 4

### e) (6,7-dimethoxyquinazolin-4-yl)(1-isopropyl-4,4-dimethyl-4,5-dihydro-1H-imidazol-2-yl)acetonitrile

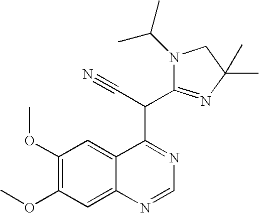

4-Chloro-6,7-dimethoxyquinazoline (500 mg, 0.223 mmol) was dissolved in dry DMF (94 mL) in a dry flask under an atmosphere of argon and 1-(isopropyl-4,4-dimethyl-4,5-dihydro-1H-imidazol-2-yl)acetonitrile (48 mg, 0.268 mmol) was added. The mixture was then cooled to 0° C., and 1.34 mL of potassium hexamethyldisilazane in tetrahydrofuran (0.500 M, 0.668 mmol) was added dropwise over 5 min. The resulting mixture was stirred for 18 h at room temperature. The entire mixture was then loaded onto a 10 g SCX column and washed with methanol (1 volume). Elution with ammonia in methanol, followed by concentration provided the crude product, which was purified by preparative HPLC/MS to provide 54 mg (66%) of (6,7-dimethoxyquinazolin-4-yl)(1-isopropyl-4,4-dimethyl-4,5-dihydro-1H-imidazol-2-yl)acetonitrile as an orange solid. [M+H]=368, LC/MS (EI) t_{R} 5.15 min (Method C), ¹H NMR (CDCl₃) δ (ppm) 11.10 (br. s, 1H), 8.68 (s, 1H), 8.22 (s, 1H), 7.15 (s, 1H), 5.12 (m, 1H), 4.00 (s, 3H), 3.96 (s, 3H), 3.30 (s, 2H), 1.40 (s, 6H), 1.32 (s, 3H), 1.30 (s, 3H).

### Example 5

### q) 6,7-dimethoxy-4-[6-(2-methoxyethoxy)-3,4-dihydroisoquinoline-2(1H)-yl]quinazoline

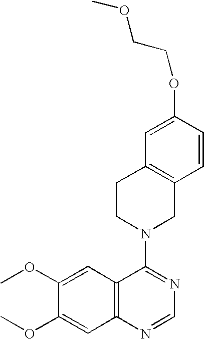

6-(2-Methoxyethoxy)-1,2,3,4-tetrahydroisoquinoline (29 mg, 0.14 mmol) was dissolved in 1.0 mL N,N-dimethylacetamide to give a clear, colorless solution. 4-Chloro-6,7-dimethoxyquinazoline (43.3 mg, 0.193 mmol) was added, resulting in the formation of a cloudy yellow suspension. Tetra-n-butylammonium iodide (16 mg, 0.043 mmol) and potassium carbonate (57.4 mg, 0.415 mmol) were subsequently added, and the reaction mixture was heated in a sealed tube at 140° C. for 2.5 hours. The reaction was concentrated to yield a brown solid. The brown solid was dissolved in ethyl acetate (30 mL) and the organic layer was washed with water (3×10 mL) and with brine (1×10 mL). The organic layer was dried over sodium sulfate, filtered, and concentrated in vacuo to provide a yellow-orange oil. Purification on a C18 column preparative (30×100 mm) HPLC column using a gradient of 20-80% acetonitrile:water (with 0.1% formic acid) and a flow rate of 45 mL/min yielded a yellow oil. The yellow oil was then loaded onto an SCX column (0.25 g), washed with methanol, eluted with 4 mL ammonia in methanol (7M), and concentrated. Dissolution in dichloromethane, followed by concentration in vacuo afforded 29.3 mg (53%) of 6,7-dimethoxy-4-[6-(2-methoxyethoxy)-3,4-dihydroisoquinoline-2(1H)-yl]quinazoline as a light yellow foam. MS [M+H]=396.2, LC/MS (EI) t_{R} 3.81 min (Method B), ¹H NMR (CDCl₃) δ (ppm) 8.18 (s, 1H), 7.19 (s, 1H), 7.10 (d, J=9.0 Hz, 1H), 6.82 (m, 2H), 4.78 (s, 2H), 4.13 (t, J=6 Hz, 2H), 4.04 (s, 3H), 4.00 (s, 3H), 3.93 (t, J=6 Hz, 2H), 3.77 (t, J=6 Hz, 2H), 3.47 (s, 3H), 3.15 (t, J=6 Hz, 2H).

The following compounds were prepared in a similar fashion with different starting materials:

### 2-{[2-(6,7-dimethoxyquinazolin-4-yl)-1,2,3,4-tetrahydroisoquinolin-7- yl]-oxy}ethanol

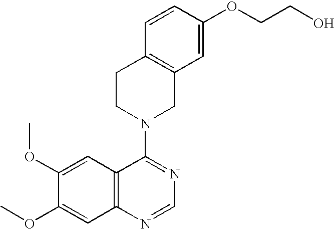

Prepared in 41% yield using 2-(1,2,3,4-tetrahydroisoquinolin-7-yloxy)ethanol. [M+H]=382.2, LC/MS (EI) t_{R} 3.5 min (Method B)

### 2-{[2-(6,7-dimethoxyquinazolin-4-yl)-1,2,3,4-tetrahydroisoquinolin-5-yl]oxy}ethanol

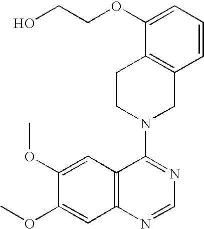

Prepared in 42% yield using 2-(1,2,3,4-tetrahydroisoquinolin-5-yloxy)ethanol. [M+H]=382.2, LC/MS (EI) t_{R} 3.6 min (Method B)

The following compounds were prepared in a similar fashion with different starting materials (no SCX column chromatography performed):

### w) Ethyl 5-(6,7-dimethoxyquinazolin-4-yl)-4,5,6,7-tetrahydro-1H-pyrazolo[4,3-c]pyridine-3-carboxylate

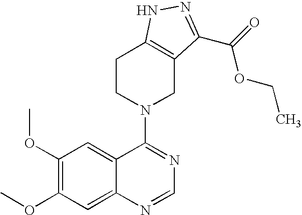

Prepared in 82% yield using ethyl-4,5,6,7-tetrahydro-1H-pyrazolo[4,3-c]pyridine-3-carboxylate. [M+H]=384.2, LC/MS (EI) t_{R} 2.74 min (Method B)

### y) N-cyclopropyl-5-(6,7-dimethoxyquinazolin-4-yl)-4,5,6,7-tetrahydro-1H-pyrazolo[4,3-c]pyridine-3-carboxamide hydroformate

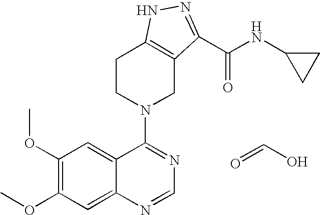

Prepared in 64% yield using N-cyclopropyl-4,5,6,7-tetrahydro-1H-pyrazolo[4,3c]pyridine-3-carboxamide trifluoroacetate. [M+H]=395.2, LC/MS (EI) t_{R} 2.5 min (Method B)

### 6,7-dimethoxy-4-(1,4,6,7-tetrahydro-5H-pyrazolo[4,3-c]pyridin-5-yl)quinazoline hydroformate

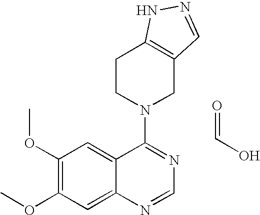

Prepared in 8% yield using 4,5,6,7-tetrahydro-1H-pyrazolo[4,3-c]pyridine dihydrochloride. [M+H]=312.2, LC/MS (EI) t_{R} 2.28 min (Method B)

### 6,7-dimethoxy-4-[8-(2-methoxyethoxy)-3,4-dihydroisoquinoline-2(1H)-yl]quinazoline

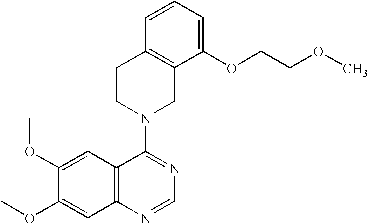

Prepared in 25% yield using 8-(2-methoxyethoxy)-1,2,3,4-tetrahydroisoquinoline hydrochloride. [M+H]=396.2, LC/MS (EI) t_{R} 4.1 min (Method B)

### 5-(6,-dimethoxyquinazolin-4-yl)-N-methyl-4,5,6,7-tetrahydro-1H-pyrazolo[4,3-c]pyridine-3-carboxamide hydroformate

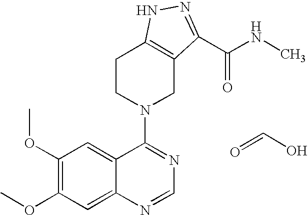

Prepared in 40% yield using N-methyl-4,5,6,7-tetrahydro-1H-pyrazolo[4,3-c]pyridine-3-carboxamide hydroformate. [M+H]=369.2, LC/MS (EI) t_{R} 2.63 mine (Method B)

### 5-(6,7-dimethoxyquinazolin-4-yl)-4,5,6,7-tetrahydro-1H-pyrazolo[4,3-c]pyridine-3-carboxamide

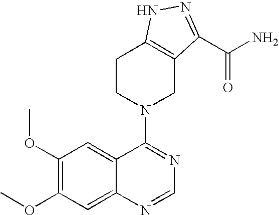

Prepared in 51% yield using 4,5,6,7-tetrahydro-1H-pyrazolo[4,3-c]pyridine-3-carboxamide hydroformate. [M+H]=355.5, LC/MS (EI) t_{R} 2.62 min (Method B)

### a) 4-(6,7-dimethoxy-3,4-dihydroisoquinoline-2(1H)-yl)-6,7-dimethoxyquinazoline

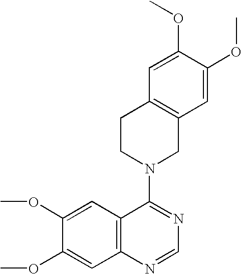

[M+H]=382.2, LC/MS (EI) t_{R} 4.8 min (Method B)

### b) 4-(6,7-dimethoxy-1-methyl-3,4-dihydroisoquinoline-2(1H)-yl)-6,7-dimethoxyquinazoline

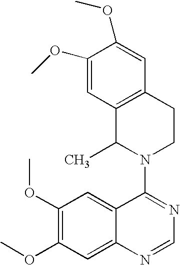

[M+H]=396.2, LC/MS (EI) t_{R} 5.07 min (Method B)

### c) 4-(1-isopropyl-6,7-dimethoxy-3,4-dihydroisoquinoline-2(1H)-yl)-6,7-dimethoxyquinazoline

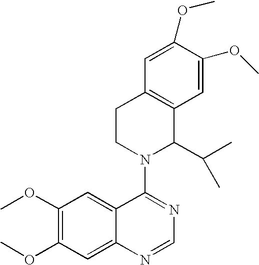

[M+H]=424.2, LC/MS (EI) t_{R} 5.35 min (Method B)

### Example 6

### p) 6,7-dimethoxy-4-[7-(2-methoxyethoxy)-3,4-dihydroisoquinoline-2(1H)-yl]quinazoline

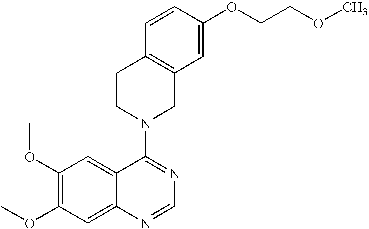

A mixture of 4-chloro-6,7-dimethoxyquinazoline (0.0851 g, 0.379 mmol), 7-(2-methoxyethoxy)-1,2,3,4-tetrahydroisoquinoline hydrochloride (0.12 g, 0.49 mmol), N,N-dimethylacetamide (3.0 mL), potassium carbonate (0.189 g, 1.37 mmol), and lithium bromide (0.0066 g, 0.076 mmol) was heated at 160° C. for 4 hr. The solvent was then evaporated and the residue was dissolved in dichloromethane (50 mL), which was washed with sodium bicarbonate (1×30 mL). The organics were concentrated and the residue was purified by HPLC followed by column chromatography (using 1-3% methanol, 0.06% ammonia in 1:1 ethyl acetate/hexane as eluent) to afford 5 mg (3%) of 6,7-dimethoxy-4-[7-(2-methoxyethoxy)-3,4-dihydroisoquinoline-2(1H)-yl]quinazoline as a white solid. ¹H NMR (CDCl₃) δ (ppm) 8.69 (s, 1H), 7.28 (s, 1H), 7.20 (s, 1H), 7.14 (d, J=8.4 Hz, 1H), 6.85 (d, J=8.4 Hz, 1H), 6.82 (s, 1H), 8.80 (s, 2H), 4.12 (m, 2H), 4.04 (s, 3H), 4.01 (s, 3H), 3.93 (t, J=5.7 Hz, 2H), 3.76 (m, 2H), 4.46 (s, 3H), 3.13 (t, J=5.7 Hz, 2H), [M+H]=396.2, LC/MS (EI) t_{R} 3.81 min (Method B)

The following compounds were prepared in a similar fashion with different starting materials:

### l) 4-(4,7-dihydrothieno[2,3-c]pyridin-6(5H)-yl)-6,7-dimethoxyquinazoline

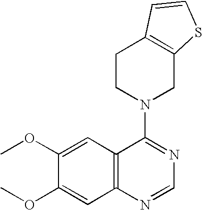

Prepared in 8% yield using 4,5,6,7-tetrahydrothieno[2,3-c]pyridine. [M+H]=328.1, LC/MS (EI) t_{R} 2.64 min (Method B)

### k) 2-(6,7-dimethoxyquinazolin-4-yl)-1,2,3,4-tetrahydroisoquinoline-6,7-diol

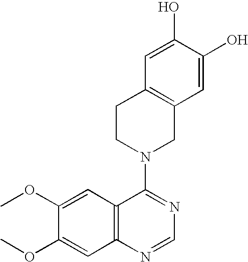

Prepared in 6% yield using 6-7-dihydroxy-1,2,3,4-tetrahydroisoquinoline. [M+H]=354.2, LC/MS (EI) t_{R} 3.53 min (Method B)

### m) 4-[(3S)-6,7-dimethoxy-3-methyl-3,4-dihydroisoquinoline-2(1H)-yl]-6,7-dimethoxyquinazoline

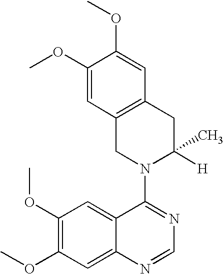

[M+H]=396.2, LC/MS (EI) t_{R} 5.39 min (Method B)

### n) 4-[(3R)-6,7-dimethoxy-3-methyl-3,4-dihydroisoquinoline-2(1H)-yl]-6,7-dimethoxyquinazoline

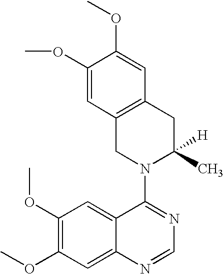

[M+H]=336.2, LC/MS (EI) t_{R} 5.38 min (Method B)

Compounds m) and n) were prepared as a racemic mixture using 6,7-dimethoxy-3-methyl-1,2,3,4-tetrahydroisoquinoline as a starting material, and separated by chromatography on a chiral SFC column using 25% methanol as eluent (10% yield of each isomer).

### i) 4-(5-bromo-1H-indazol-1-yl)-6,7-dimethoxyquinazoline

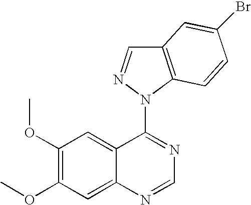

[M+H]=385, LC/MS (EI) t_{R} 6.1 min (Method B)

### j) 4-(5-bromo-3H-indazol-3-yl)-6,7-dimethoxyquinazoline

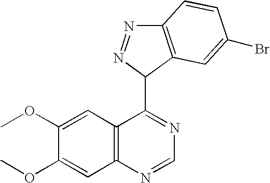

[M+H]=385, LC/MS (EI) t_{R} 6.05 min (Method B)

Compounds i) and j) were prepared as a mixture using 5-bromo-1H-indazole as a starting material, and separated by crystallization and chromatography. Yields were 23% and 2%, respectively)

The following compound was prepared in a similar fashion with different starting materials (no lithium bromide was added to the reaction):

### g) 4-(1,3-dihydro-2H-isoindol-2-yl)-6,7-dimethoxyquinazoline

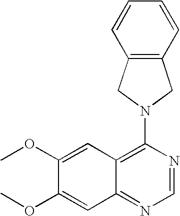

Prepared in 32% yield using isoindoline. [M+H]=308.1, LC/MS (EI) t_{R} 5.08 min (Method B)

### Example 7

### t) 1-benzyl-3-(6,7-dimethoxyquinazolin-4-yl)imidazolidin-4-one

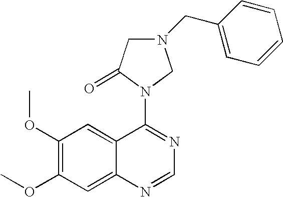

1-Benzyl-imidazolidin-4-one (0.051 g, 0.29 mmol) in N,N-dimethylacetamide (3 mL) was treated with sodium hydride (0.013 g, 0.33 mmol) at room temperature for 30 minutes. 4-chloro-6,7-dimethoxyquinazoline (0.050 g, 0.22 mmol) and copper(I) iodide (0.008 g, 0.04 mmol) were then added and the mixture was stirred at 130° C. for 2 hr, then cooled and quenched by adding water. The solvent was evaporated in vacuo, and the resulting residue was extracted with dichloromethane (100 mL). The organics were filtered and the solution was washed with water (50 mL) and concentrated. The residue was purified by column chromatography (using 3% methanol in 1:1 ethyl acetate/hexane, ammonia 0.05%) followed by preparative HPLC to afford 3 mg (4%) of 1-benzyl-3-(6,7-dimethoxyquinazolin-4-yl)imidazolidin-4-one as a give light yellow solid. ¹H NMR (CDCl₃), δ (ppm) 8.82 (s, 1H), 7.59 (s, 1H), 7.50-7.27 (m, 5H), 4.71 (m, 2H), 4.31 (m, 2H), 4.06 (s, 3H), 4.00 (s, 3H), 3.87 (s, 2H), [M+H]=365.2, LC/MS (EI) t_{R} 3.94 min (Method B).

The following compound was prepared in a similar fashion with different starting materials:

### r) 2-(6,7-dimethoxyquinazolin-4-yl)-6,7-dimethoxy-3,4-dihydroisoquinoline-1(2H)-one

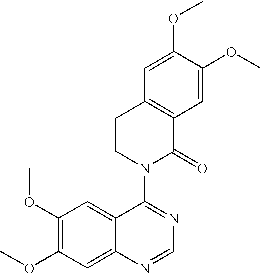

Prepared in 9% yield using 6,7-dimethoxy-3,4-dihydro-2H-isoquinolin-1-one. [M+H]=396.2, LC/MS (EI) t_{R} 5.35 min (Method B).

The following compound was prepared in a similar fashion with different starting materials (tetra-n-butyl ammonium iodide was used in place of copper iodide):

### s) 2-(6,7-dimethoxyquinazolin-4-yl)-5-(2-methoxyethoxy)-3,4-dihydroisoquinoline-1 (2H)-one

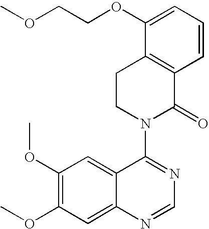

Prepared in 5% yield using 5-(2-methoxyethoxy)-3,4-dihydroisoquinoline-1(2H)-one. [M+H]=410.2, LC/MS (EI) t_{R} 5.67 min (Method B)

### Example 9

### u) 4-(1-benzyl-1H-pyrazol-4-yl)-6,7-dimethoxyquinazoline hydroformate

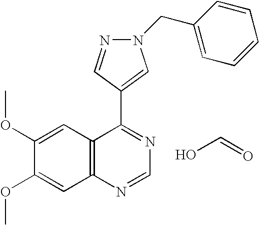

4-Chloro-6,7-dimethoxyquinazoline (100 mg, 0.0004 mol), bis(triphenylphosphine)palladium(II) chloride (54.7 mg, 0.008 mmol), 1-benzyl-1H-pyrazole-4-boronic acid (130 mg, 0.00067 mol), 0.16 mL of 2.00 M of sodium carbonate in water and 2 mL of dimethoxyethane:water:ethanol (7:3:2) were combined in a 10 mL sealed tube. The reaction was subjected to microwave irradiation at 300 watts, 140° C. for 600 seconds. The reaction contents were filtered through a pad of celite using methanol and then concentrated. The residue was purified by ISCO chromatography with 50% ethyl acetate:hexane followed by 70:30:1 ethyl acetate/methanol/ammonia to give 111 mg of 4-(1-benzyl-1H-pyrazol-4-yl)-6,7-dimethoxyquinazoline hydroformate as a yellow solid. An additional 10 mg of crude product was purified by preparative HPLC using a gradient of 20-80% acetonitrile (0.1% formic acid). Overall yield 113 mg (70%). MS [M+H]=347.2, LC/MS (EI) t_{R} 5.72 min (Method B), ¹H NMR (CDCl₃) δ (ppm) ¹H NMR 9.05 (s, 1H), 8.16 (s, 1H), 8.07 (s, 1H), 7.48 (s, 1H), 7.40-7.34 (m, 5H), 7.32 (s, 1H), 5.42 (s, 2H), 4.06 (s, 3H), 3.96 (s, 3H).

### Example 10

### o) 6,7-dimethoxy-4-[5-(2-methoxyethoxy)-3,4-dihydroisoquinoline-2(1H)-yl] quinazoline

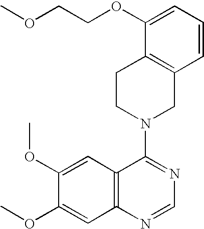

4-Chloro-6,7-dimethoxyquinazoline (44.5 mg, 0.198 mmol) was added to a solution of 5-(2-methoxyethoxy)-1,2,3,4-tetrahydroisoquinoline (30.5 mg, 0.147 mmol) in N,N-dimethylacetamide (1.0 mL, 0.011 mol) to afford a cloudy yellow suspension. Sodium iodide (10 mg, 0.07 mmol) and potassium carbonate (55.9 mg, 0.404 mmol) were added and the reaction was heated in a sealed tube at 160° C. for 2.75 hours. Volatiles were removed in vacuo to afford a brown oil. Purification on a Berger Mini-Gram SFC (using 17% methanol at a wavelength of 325 nm and a flow rate of 9.9 mL/min on a 7.8 mm i.d. pyridine column) afforded 12.5 mg (21.5%) of 6,7-dimethoxy-4-[5-(2-methoxyethoxy)-3,4-dihydroisoquinoline-2(1H)-yl]quinazoline as a yellow oil. ¹H NMR (CDCl₃), d (ppm) 8.70 (s, 1H), 7.21 (s, 1H), 7.19 (d, J=7.5 Hz, 1H), 6.84 (d, J=6 Hz, 1H), 6.765 (d, J=9 Hz, 1H), 4.79 (s, 2H), 4.19 (t, J=7.5 Hz, 2H), 4.04 (s, 3H), 4.02 (s, 3H), 3.90 (t, J=6 Hz, 2H), 3.81 (t, J=6 Hz, 2H), 3.48 (s, 3H), 3.13 (t, J=6 Hz, 2H), [M+H]=396.2, LC/MS (EI) t_{R} 4.2 min (Method B).

### Example 11

### 6,7-dimethoxy-4-(1,4,5,7-tetrahydro-6H-pyrazolo[3,4-c]pyridin-6-yl)quinazoline

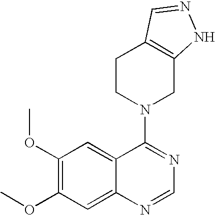

4,5,6,7-Tetrahydro-1H-pyrazolo[3,4-c]pyridine dihydrochloride (0.450 g, 2.29 mmol) in N,N-dimethylacetamide (110.00 mL) was treated with N,N-diisopropylethylamine (1.74 mL, 9.97 mmol) at 100° C. for 5 minutes. 4-chloro-6,7-dimethoxyquinazoline (0.448 g, 1.99 mmol) and tetra-n-butylammonium iodide (0.0560 g, 0.152 mmol) were then added and the mixture was heated at 120° C. for 6 hours. The solvent was evaporated and the residue was diluted with 10% methanol/dichloromethane (60 mL) and filtered. The filtrate was concentrated and purified by column chromatography (using 4-10% methanol/dichloromethane) to afford 250 mg (40%) of 6,7-dimethoxy-4-(1,4,5,7-tetrahydro-6H-pyrazolo[3,4-c]pyridin-6-yl)quinazoline as a yellow solid. ¹H NMR (DMSO) δ (ppm) 8.87 (s, 1H), 8.86 (s, 1H), 8.52 (s, 1H), 7.36 (s, 1H), 3.99 (s, 3H), 3.90 (s, 3H), 3.83 (s, 2H), 2.99 (t, J=5.7 Hz, 2H), 2.75 (t, J=5.7 Hz, 2H), [M+H]=312.1, LC/MS (EI) t_{R} 3.91 min (Method I).

### Example 12

### f) 4-(6,7-dimethoxy-3-methyl-3,4-dihydroisoquinoline-2(1H)-yl)-6,7-dimethoxyquinazoline

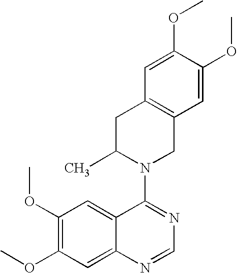

A mixture of 4-chloro-6,7-dimethoxyquinazoline (0.045 g, 0.20 mmol), xxx (0.054 g, 0.22 mmol), N,N-diisopropylamine (0.15 mL), and N,N-dimethylacetamide (2.00 mL) was subjected to microwave irradiation at 200° C. for 1000 seconds. The solvent was then evaporated and the residue was dissolved in ethyl acetate (30 mL). The organics were washed with sodium bicarbonate (2×30 mL) and concentrated. The residue was purified by chromatography (using 1.5% methanol/dichloromethane). A second chromatographic purification (using 3% methanol, 0.06% ammonia in ethyl acetate/hexane 1:1) afforded 28 mg (35%) of 4-(6,7-dimethoxy-3-methyl-3,4-dihydroisoquinoline-2(1H)-yl)-6,7-dimethoxyquinazoline. ¹H NMR (CDCl₃) δ (ppm) 8.67 (s, 1H), 7.25 (s, 1H), 7.17 (s, 1H), 6.68 (s, 1H), 6.57 (s, 1H), 4.82 (m, 1H), 4.75 (s, 2H), 4.03 (s, 3H), 4.02 (s, 3H), 3.88 (s, 3H), 3.87 (s, 3H), 3.47 (m, 1H), 2.65 (d, 1H), 1.24 (d, J=6.6 Hz, 3H), [M+H]=396.2, LC/MS (EI) t_{R} 5.2 min (Method B).

The following compound was prepared in a similar fashion with different starting materials:
h) (3S)-2-(6,7-dimethoxyquinazolin-4-yl)-6,7-dimethoxy-1,2,3,4-tetrahydroisoquinoline-3-carboxylic acid

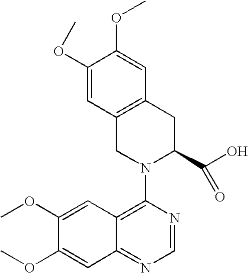

Prepared in 16% yield using (S)-(-)-1,2,3,4-tetrahydro-6,7-dimethoxy-3-isoquinolinecarboxylic acid p-toluesulfonic acid salt and 10 equivalents of potassium carbonate, with a reaction time of 5 hours at 195° C.; the product was purified by column chromatography using 10% methanol/dichloromethane. [M+H]=426.1, LC/MS (EI) t_{R} 4.62 min (Method B).

### Example 13

### z) N-(cyclopropylmethyl)-5-(6,7-dimethoxyquinazolin-4-yl)-4,5,6,7-tetrahydro-1H-pyrazolo[4,3-c]pyridine-3-carboxamide hydroformate

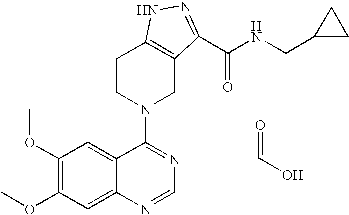

Tert-butyl 3-{[(cyclopropylmethyl)amino]carbonyl}-1,4,6,7-tetrahydro-5H-pyrazolo[4,3-c]pyridine-5-carboxylate (0.042 g, 0.13 mmol) was treated with trifluoroacetic acid (2.0 mL, 30%, 0.0078 mol) in dichloromethane for 4 hours at room temperature. The solvent was then evaporated in vacuo and the residue was triturated with ethyl ether. The resulting white solid was dissolved in N,N-dimethylacetamide (3.00 mL) and 4-chloro-6,7-dimethoxyquinazoline (0.022 g, 0.098 mmol), tetra-n-butylammonium iodide (0.0054 g, 0.015 mmol) and potassium carbonate (0.027 g, 0.20 mol) were added. The mixture was heated at 120° C. for 3 hours. The solvent was then evaporated and the residue was diluted with 10% methanol/dichloromethane (60 mL), which was then filtered and concentrated. The residue was purified by preparative HPLC to afford 18 mg (45%) of N-(cyclopropylmethyl)-5-(6,7-dimethoxyquinazolin-4-yl)-4,5,6,7-tetrahydro-1H-pyrazolo[4,3-c]pyridine-3-carboxamide hydroformate as a white solid. ¹H NMR (MeOD) δ (ppm) 8.59 (s, 1H), 8.10 (s, 1H), 7.47 (s, 1H), 7.16 (s, 1H), 5.31 (s, 2H), 4.34 (t, J=5.7 Hz, 2H), 4.07 (s, 3H), 4.04 (s, 3H), 3.21 (m, 2H), 3.12 (t, J=5.4 Hz, 2H), 1.07 (m, 1H), 0.53 (m, 2H), 0.26 (m, 2H), [M+H]=409.2, LC/MS (EI) t_{R} 3.35 min (Method B).

### Example 14

### v) Synthesis of 12-(6,7-dimethoxyquinazolin-4-yl)-1,2,3,4-tetrahydroisoquinolin-6-yl]methanol

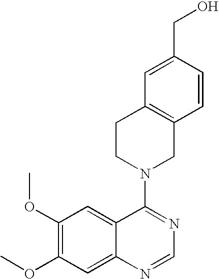 Step 1

A mixture of 4-chloro-6,7-dimethoxyquinazoline (0.50 g, 0.0022 mol), 6-methoxycarbonyl-1,2,3,4-tetrahydroisoquinoline hydrochloride (0.660 g, 2.90 mmol), potassium carbonate (0.923 g, 6.68 mmol) and N,N-dimethylacetamide (15 mL) was heated at 120° C. for 2 hours. The solvent was then evaporated, and the residue was diluted with ethyl acetate (100 mL) and washed with sodium bicarbonate (2×50 mL). The organics were separated and concentrated and the residue was purified by column chromatography (using 3% methanol in ethyl acetate/hexane 1:1, ammonia 0.03%) to afford 675 mg (80%) of methyl 2-(6,7-dimethoxyquinazolin-4-yl)-1,2,3,4 tetrahydroisoquinoline-6-carboxylate as a pale yellow solid.

Step 2

Lithium tetrahydroaluminate (0.0205 g, 0.540 mmol) was added to a solution of methyl 2-(6,7-dimethoxyquinazolin-4-yl)-1,2,3,4-tetrahydroisoquinoline-6-carboxylate (0.205 g, 0.540 mmol, prepared as described in Step 1 above) in tetrahydrofuran (8 mL) and a small amount of dichloromethane (added to aid dissolution of the ester) at room temperature After 30 minutes, ethyl alcohol (8 mL) and water (2 mL) were added. The resulting mixture was filtered through celite and the solvent was evaporated in vacuo. The residue was purified by column chromatography (5% methanol/dichloromethane) to afford 160 mg (84.3%) of [2-(6,7-dimethoxyquinazolin-4-yl)-1,2,3,4-tetrahydroisoquinolin-6-yl]methanol as a white solid. ¹H NMR (CDCl₃) δ (ppm) 8.68 (s, 1H), 7.28 (s, 1H), 7.26 (s, 1H), 7.21 (m, 3H), 4.84 (s, 2H), 4.70 (s, 2H), 4.04 (s, 3H), 4.01 (s, 3H), 3.95 (t, J=5.7 Hz, 2H), 3.20 (t, J=5.7 Hz, 2H), MS [M+1] 352.2; LC/MS (EI) t_{R} 2.54 min (Method B).

The following compound was prepared in a similar fashion with different starting materials:

### [5-(6,7-dimethoxyquinazolin-4-yl)-4,5,6,7-tetrahydro-1H-pyrazolo[4,3- c]pyridin-3-yl]methanol

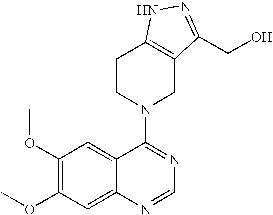

Prepared in 94% yield from ethyl 5-(6,7-dimethoxyquinazolin-4-yl)-4,5,6,7-tetrahydro-1H-pyrazolo[4,3-c]pyridine-3-carboxylate. MS [M+1] 342.1, LC/MS (EI) t_{R} 2.81 min (Method B).

### Example 15

### 6,7-dimethoxy-4-[6-(methoxymethyl)-3,4-dihydroisoquinoline-2(1H)-yl]quinazoline

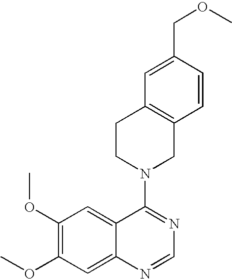

[2-(6,7-Dimethoxyquinazolin-4-yl)-1,2,3,4-tetrahydroisoquinolin-6-yl]methanol (0.025 g, 0.071 mmol) in N,N-dimethylacetamide (3.0 mL) was treated with sodium hydride (0.0085 g, 0.21 mmol) at room temperature for 30 minutes. Methyl iodide (13 μL, 0.21 mmol) was added and the mixture was stirred for 1 hour. Dimethylamine (100 μL) was then added and stirred for an additional 20 minutes. The solvent was evaporated in vacuo, and the residue was dissolved in ethyl acetate (50 mL) and washed with sodium bicarbonate (2×30 mL). The organics were concentrated and the residue was purified by column chromatography (using 1-2% methanol in 1/1 ethyl acetate/hexane, ammonia 0.03%) to afford 8 mg (30%) of 6,7-dimethoxy-4-[6-(methoxymethyl)-3,4-dihydroisoquinoline-2(1H)-yl]quinazoline. [M+H]=312.2, LC/MS (EI) t_{R} 2.28 min (Method B).

### Example 16

### 6,7-dimethoxy-4-(1-methyl-1,4,5,7-tetrahydro-6H-pyrazolo[3,4-c]pyridin-6-yl)quinazoline

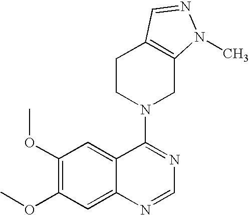

A mixture of 6,7-dimethoxy-4-(1,4,5,7-tetrahydro-6H-pyrazolo[4,3-c]pyridin-5-yl)quinazoline (30 mg, 0.096 mmol), methyl iodide (9.00 μL, 0.14 mmol), N,N-dimethylacetamide (2.00 mL), and potassium carbonate (40.0 mg, 0.289 mmol) was stirred at 80° C. for 2 hours. The solvent was then removed in vacuo and the residue was diluted with 10% methanol/dichloromethane (30 mL) and water (5 mL). The organics were separated and concentrated and the residue was purified by preparative HPLC to afford 3 mg (10%) of 6,7-dimethoxy-4-(1-methyl-1,4,5,7-tetrahydro-6H-pyrazolo[3,4-c]pyridin-6-yl)quinazoline. [M+H]=326.1, LC/MS (EI) t_{R} 4.34 min (Method I). (10% of 6,7-dimethoxy-4-(2-methyl-2,4,5,7-tetrahydro-6H-pyrazolo[3,4-c]pyridine-6-yl)quinazoline was also produced during the reaction).

The following compounds were prepared in a similar fashion with different starting materials:

### 4-(1-ethyl-1,4,5,7-tetrahydro-6H-pyrazolo[3,4-c]pyridin-6-yl)-6,7-dimethoxyquinazoline

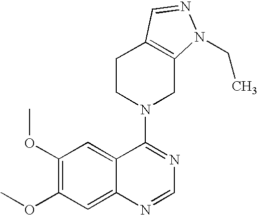

Prepared in 20% yield using iodoethane. [M+H]=340.1, LC/MS (EI) t_{R} 4.49 min (Method I). (20% of 4-(2-ethyl-2,4,5,7-tetrahydro-6H-pyrazolo[3,4-c]pyridine-6-yl)-6,7-dimethoxyquinazoline was also produced during the reaction).

### 4-(1-benzyl-1,4,5,7-tetrahydro-6H-pyrazolo[3,4-c]pyridin-6-yl)-6,7-dimethoxyquinazoline

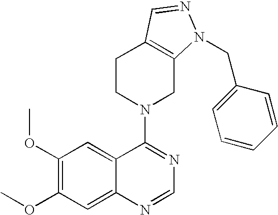

Prepared in 10% yield using (iodomethyl)benzene. [M+H]=402.1, LC/MS (EI) t_{R}4.96 min (Method I). (10% of 4-(2-benzyl-2,4,5,7-tetrahydro-6H-pyrazolo[3,4-c]pyridine-6-yl)-6,7-dimethoxyquinazoline was also produced during the reaction).

### 4-(1,3-dimethyl- 1,4,5,7-tetrahydro-6H-pyrazolo[3,4-c]pyridin-6-yl)-6,7-dimethoxyquinazoline

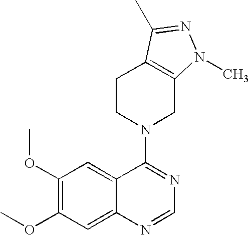

Prepared in 49% yield using 4 equivalents of methyl iodide. [M+H]=340.1, LC/MS (EI) t_{R} 4.67 min (Method J).

The following compound was prepared in a similar fashion with materials:

### Ethyl 5-(6,7-dimethoxyquinazolin-4-yl)-1-ethyl-4,5,6,7-tetrahydro-1H-pyrazolo[4,3-c]pyridine-3-carboxylate

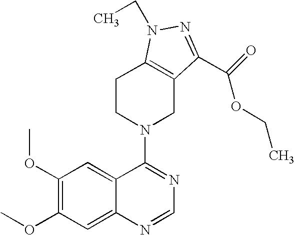

Prepared in 39% yield using ethyl 5-(6,7-dimethoxyquinazolin-4-yl)-4,5,6,7-tetrahydro-1H-pyrazolo[4,3-c]pyridine-3-carboxylate and iodoethane. [M+H]=412.2, LC/MS (EI) t_{R} 4.19 min (Method B).

### Example 17

### x) 5-(6,7-dimethoxyquinazolin-4-yl)-4,5,6,7-tetrahydro-1H-pyrazolo[4,3-c]pyridine-3-carboxylic acid trifluoroacetate

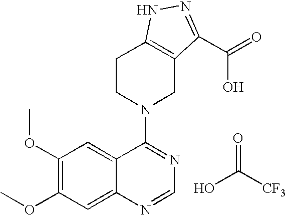

An aqueous solution of lithium hydroxide (0.8 M, 3.75 mL) was added to a solution of ethyl-5-(6,7-dimethoxyquinazolin-4-yl)-4,5,6,7-tetrahydro-1H-pyrazolo[4,3-c]pyridine-3-carboxylate (0.050 g, 0.13 mmol) in 1,4-dioxane (10 mL). The resulting mixture was stirred for 24 hours, then the solvent was then evaporated in vacuo. The residue was diluted with 20% methanol/dichloromethane (60 mL), acidified using trifluoroacetic acid, and filtered. The solution was concentrated and purified by column chromatography (10-20% methanol in dichloromethane) to afford 42 mg (91%) of 5-(6,7-dimethoxyquinazolin-4-yl)-4,5,6,7-tetrahydro-1H-pyrazolo[4,3-c]pyridine-3-carboxylic acid trifluoroacetate as a white solid. MS [M+H]=356.2, LC/MS (EI) t_{R} 2.54 min (Method B), ¹H NMR (DMSO) δ (ppm) ¹H NMR 8.52 (s, 1H), 7.21 (s, 2H), 5.75 (s, 2H), 4.79 (s, 2H), 3.96 (s, 3H), 3.89 (s, 3H), 3.03 (b, 2H).

The following compounds were prepared in a similar fashion with different starting materials:

### 2-(6,7-dimethoxyquinazolin-4-yl)-1,2,3,4-tetrahydroisoquinoline-6-carboxylic acid trifluoroacetate

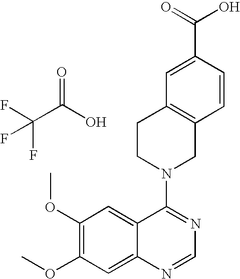

Prepared using methyl 2-(6,7-dimethoxyquinazolin-4-yl)-1,2,3,4-tetrahydroisoquinoline-6-carboxylate. [M+H]=366.2, LC/MS (EI) t_{R} 2.99 min (Method B).

### 2-(6,7-dimethoxyquinazolin-4-yl)-1,2,3,4-tetrahydroisoquinoline-6-carboxylic acid

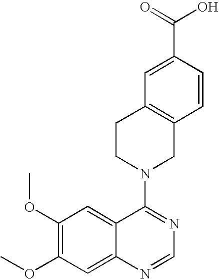

Prepared in 46% yield using methyl 2-(6,7-dimethoxyquinazolin-4-yl)-1,2,3,4-tetrahydroisoquinoline-6-carboxylate. [M+H]=366.2, LC/MS (EI) t_{R} 2.99 min (Method B).

### Example 18

### N-cyclopropyl-2-(6,7-dimethoxyquinazolin-4-yl)-1,2,3,4-tetrahydroisoquinoline-6-carboxamide hydroformate

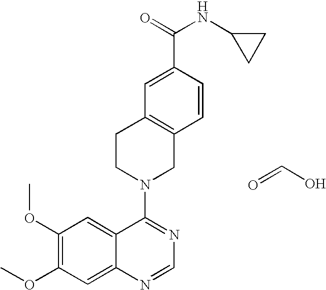

A mixture of 2-(6,7-dimethoxyquinazolin-4-yl)-1,2,3,4-tetrahydroisoquinoline-6-carboxylic acid (42 mg, 0.11 mmol), cyclopropylamine (16 μL, 0.23 mmol), N,N-diisopropylcarbodiimide (35 μL, 0.22 mmol), 1-hydroxybenzotriazole (8 mg, 0.06 mmol) and N,N-dimethylformamide (3.0 mL) was stirred for 14 hours at room temperature, then the solvent was evaporated. The residue was dissolved in dichloromethane (30 mL) and washed with sodium bicarbonate (25 mL). The organics were concentrated and the residue was purified by column chromatography (using 5% methanol/dichloromethane). Additional purification by preparative HPLC afforded 24 mg (52%) of N-cyclopropyl-2-(6,7-dimethoxyquinazolin-4-yl)-1,2,3,4-tetrahydroisoquinoline-6-carboxamide hydroformate as a white solid. ¹H NMR (CDCl₃) δ (ppm) 8.66 (s, 1H), 7.66 (s, 1H), 7.54 (d, J=8.0 Hz, 1H), 7.37 (s, 1H), 7.22 (d, J=8.0 Hz, 1H), 7.16 (s, 1H), 5.85 (b, 1H), 4.88 (s, 2H), 4.03 (s, 3H), 4.00 (s, 3H), 3.97-3.88 (m, 5H), 3.19 (t, J=5.1 Hz, 2H, 2.90 (m, 1H), 0.86 (m, 2H), 0.63 (m, 2H), MS [M+H]=405.2, LC/MS (EI) t_{R} 3.37 min (Method B).

The following compound was prepared in a similar fashion with different starting materials:

### N-(cyclopropylmethyl)-2-(6,7-dimethoxyquinazolin-4-yl)-1,2,3,4-tetrahydroisoquinoline-6-carboxamide hydroformate

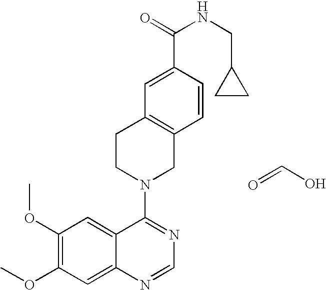

Prepared in 58% yield using cyclopropylmethylamine. [M+H]=419.2, LC/MS (EI) t_{R} 3.71 min (Method B).

### Example 19

### Synthesis of [5-(6,7-dimethoxyquinazolin-4-yl)-1-methyl-4,5,6,7-tetrahydro-1H-pyrazolo [4,3-c]pyridin-3-yl]methanol

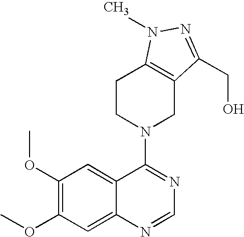 Step 1

Lithium tetrahydroaluminate (0.0990 g, 2.61 mmol) was added to a solution of ethyl 5-(6,7-dimethoxyquinazolin-4-yl)-4,5,6,7-tetrahydro-1H-pyrazolo[4,3-c]pyridine-3-carboxylate (0.5000 g, 1.304 mmol) in tetrahydrofuran (20 mL) at room temperature. After 30 minutes, methanol/dichloromethane (20%, 30 mL) and water (2 mL) were added. The resulting mixture was filtered and the solvent was removed in vacuo. The residue was purified by column chromatography (using 10-20% methanol/ethyl acetate) to afford 375 mg (84.2%) of [5-(6,7-dimethoxyquinazolin-4-yl)-4,5,6,7-tetrahydro-1H-pyrazolo[4,3-c]pyridin-3-yl]methanol as a white solid. MS [M+H]=342.1, LC/MS (EI) t_{R} 2.79 min (Method B).

Step 2

[5-(6,7-Dimethoxyquinazolin-4-yl)-4,5,6,7-tetrahydro-1H-pyrazolo[4,3-c]pyridin-3-yl]methanol (0.050 g, 0.15 mmol, prepared as described above in Step 1) in dimethylacetamide was treated with sodium tert-butoxide (18.3 mg, 0.190 mmol) for 30 min at room temperature. Methyl iodide (0.012 mL, 0.19 mmol) was then added and the mixture stirred for an additional 3 hours. The solvent was then evaporated and the residue was diluted with dichloromethane (40 mL) and water (30 mL). The organic layer was separated and concentrated. The residue was purified by preparative HPLC to afford 15 mg (29%) of a mixture of 1- and 2- substituted products. MS [M+H]=356.2, LC/MS (EI) t_{R} 2.73 min (Method B).

### Example 20

### 2-[5-(6,7-dimethoxyquinazolin-4-yl)-4,5,6,7-tetrahydro-1H-pyrazolo[4,3-c]pyridin-3-yl]propan-2-ol

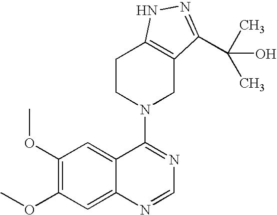

A mixture of ethyl 5-(6,7-dimethoxyquinazolin-4-yl)-4,5,6,7-tetrahydro-1H-pyrazolo[4,3-c]pyridine-3-carboxylate (128 mg, 0.334 mmol), methylmagnesium chloride (1.7 mmol) and tetrahydrofuran (5.5 mL) was stirred at 60° C. for one hour. Water (0.050 mL) was then added after the mixture was cooled to 20° C. Methanol/dichloromethane (5 mL, 20%) was then added and the mixture was filtered. The organics were concentrated and the residue was purified by column chromatography (using 5-15% methanol/ethyl acetate). Additional purification by preparative HPLC afforded 92 mg (74%) of 2-[5-(6,7-dimethoxyquinazolin-4-yl)-4,5,6,7-tetrahydro- H-pyrazolo[4,3-c]pyridin-3-yl]propan-2-ol as a white solid. ¹H NMR (CDCl₃), δ 8.54 (s, 1H), 8.09 (s, 1H), 7.34 (s, 1H), 7.17 (s, 1H), 4.91 (s, 2H), 4.05 (m, 2H), 4.01 (s, 3H), 3.97 (s, 3H), 3.04 (m, 2H), 1.54 (s, 6H), MS [M+H]=370.1, LC/MS (EI) t_{R} 4.42 min (Method I).

### Example 21

### 4-(3-isopropenyl-1,4,6,7-tetrahydro-5H-pyrazolo[4,3-c]pyridin-5-yl)-6,7-dimethoxyquinazoline

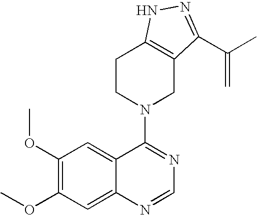

Bis(2-methoxyethyl)aminosulfur trifluoride (0.032 g, 0.00015 mol) was added to a mixture of 2-[5-(6,7-dimethoxyquinazolin-4-yl)-4,5,6,7-tetrahydro-1H-pyrazolo[4,3-c]pyridin-3-yl]propan-2-ol (0.018 g, 0.049 mmol) in methylene chloride (3.0 mL) and the resulting mixture was stirred at room temperature for 3 hours. Methanol (0.1 mL) was added, followed by the addition of ethyl acetate (30 mL). The organic layer was washed with aqueous sodium bicarbonate (1×20 mL), separated and concentrated. The residue was purified by preparative HPLC to afford 1 mg (6%) of 4-(3-isopropenyl-1,4,6,7-tetrahydro-5H-pyrazolo[4,3-c]pyridin-5-yl)-6,7-dimethoxyquinazoline. MS [M+H]=352.2, LC/MS (EI) t_{R} 4.51 min (Method I).

### Example 22

### 6,7-dimethoxy-4-[1-(3-methylbutanoyl)-1,4,6,7-tetrahydro-5H-pyrazolo[4,3-c]pyridin-5-yl]quinazoline hydroformate

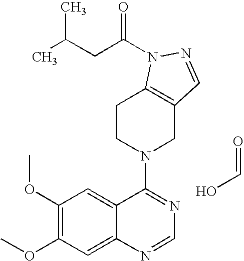

A mixture of 6,7-dimethoxy-4-(1,4,6,7-tetrahydro-5H-pyrazolo[4,3-c]pyridin-5-yl)quinazoline (10 mg, 0.032 mmol), 3-methylbutanoyl chloride (19.4 mg, 0.16 mmol), N,N-diisopropylethylamine (0.028 mL), and N,N-dimethylformamide (2.00 mL) was heated at 80° C. for 5 hours. The solvent was then evaporated and the residue was dissolved in dichloromethane (40 mL). The product was washed with sodium bicarbonate solution (30 mL) and the organics were concentrated. The residue was purified by column chromatography (using 5-15% methanol/dichloromethane). Additional purification by preparative HPLC afforded 3 mg (20%) of 6,7-dimethoxy-4-[1-(3-methylbutanoyl)-1,4,6,7-tetrahydro-5H-pyrazolo[4,3-c]pyridin-5-yl]quinazoline hydroformate as a white solid. ¹H NMR (CDCl₃), δ (ppm) 8.69 (s, 1H), 8.11 (s, 1H), 7.33 (s, 1H), 7.15 (s, 1H), 4.76 (s, 2H), 4.05 (s, 3H), 4.02 (s, 3H), 3.97 (m, 2H), 3.19 (m, 2H), 2.99 (d, J=6.6 Hz, 2H), 2.33 (m, 1H), 1.04 (d, J=6.3 Hz, 6H), MS [M+H]=396.2, LC/MS (EI) t_{R} 4.24 min (Method B).

### Example 23

### Synthesis of N-{[5-(6,7-dimethoxyquinazolin-4-yl)-4,5,6,7-tetrahydro-1H-pyrazolo[4,3-c]pyridin-3-yl]methyl}-N-ethylethanamine

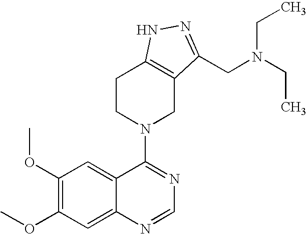 Step 1

A mixture of [5-(6,7-dimethoxyquinazolin-4-yl)-4,5,6,7-tetrahydro-1H-pyrazolo[4,3-c]pyridin-3-yl]methanol (100 mg, 0.293 mmol, prepared as described above in Example 19, Step 1), thionyl chloride (1.00 mL, 0.0137 mol) and tetrahydrofuran (0.5 mL) was heated at 80° C. for 2 hours. The solvent was removed by evaporation and the residue was purified by preparative HPLC to afford 25 mg (24%) of 4-[3-(chloromethyl)-1,4,6,7-tetrahydro-5H-pyrazolo[4,3-c]pyridin-5-yl]-6,7-dimethoxyquinazoline as a white solid. MS [M+H]=360.1, LC/MS (EI) t_{R} 4.48 min.

Step 2

A mixture of 4-[3-(chloromethyl)-1,4,6,7-tetrahydro-5H-pyrazolo[4,3-c]pyridin-5-yl]-6,7-dimethoxyquinazoline (20 mg, 0.056 mmol, prepared as described above in Step 1), N-ethylethanamine (0.2 mL, 1.93 mmol) and tetrahydrofuran (1.00 mL) was heated at 80° C. for 2 hours. The solvent was then evaporated and the resulting residue was purified by preparative HPLC to afford 16 mg (72%) of N-{[5-(6,7-dimethoxyquinazolin-4-yl)-4,5,6,7-tetrahydro-1H-pyrazolo[4,3-c]pyridin-3-yl]methyl}-N-ethylethanamine as a white solid. ¹H NMR (CDCl₃), δ (ppm) 8.68 (s, 1H), 7.26 (s, 1H), 7.19 (s, 1H), 4.65 (s, 2H), 4.04 (s, 3H), 4.01 (s, 3H), 3.93 (t, J=5.4 Hz, 2H), 3.61 (s, 2H), 3.16 (t, J=5.4 Hz, 2H), 2.53 (q, J=6.3 Hz, 4H), 1.02 (t, J=6.3 Hz, 6H), MS [M+H]=397.2, LC/MS (EI) t_{R} 3.07 min (Method B).

### Example 24

### Synthesis of 2-[5-(6,7-dimethoxyquinazolin-4-yl)-4,5,6,7-tetrahydro-1H-pyrazolo 14,3-c]pyridin-3-yl] propan-2-ol hydroformate

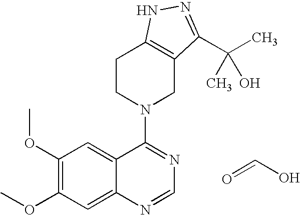 Step 1

Ethyl 5-(6,7-dimethoxyquinazolin-4-yl)-4,5,6,7-tetrahydro-1H-pyrazolo[4,3-c]pyridine-3-carboxylate (0.0770 g, 0.201 mmol) in N,N-dimethylacetamide (3.00 mL) was treated with sodium hydride (0.00964 g, 0.402 mmol) for 30 minutes at room temperature. [β-(Trimethylsilyl)ethoxy]methyl chloride (0.0670 g, 0.402 mmol) was then added and the temperature was raised to 60° C. After 3 hours, the reaction was cooled to room temperature and quenched by the addition of water (0.1 mL). The solvent was evaporated in vacuo and the resulting residue was diluted with ethyl acetate (30 mL) and washed with aqueous sodium bicarbonate solution (2×20 mL). The organics were concentrated and the residue was purified by column chromatography (using 1-3% methanol in 1:1 ethyl acetate/hexane, ammonia 0.03%) to afford 27 mg (26%) of ethyl 5-(6,7-dimethoxyquinazolin-4-yl)-1-{[2-(trimethylsilyl)ethoxy]methyl}4,5,6,7-tetrahydro-1H-pyrazolo[4,3-c]pyridine-3-carboxylate as a light yellow gum. MS [M+H]=514.20, LC/MS (EI) t_{R} 5.19 min.

Step 2

Ethyl 5-(6,7-dimethoxycinnolin-4-yl)-1-{[2-(trimethylsilyl)ethoxy]methyl}-4,5,6,7-tetrahydro-1H-pyrazolo[4,3-c]pyridine-3-carboxylate (26 mg, 0.000051 mol, prepared as describe above in Step 1) was treated with methylmagnesium chloride (3.0 M in tetrahydrofuran, 0.50 mL) at 60 degrees ° C. for one hour. After cooling to 20° C., the reaction was quenched by the addition of methanol/water (0.5 mL, 80%) and ethyl acetate (20 mL). After stirring for 10 minutes, the mixture was filtered through celite. The solution was washed with aqueous sodium bicarbonate solution (2×15 mL) and the organics were concentrated to afford 24 mg (99%) of 2-(5-(6,7-dimethoxyquinazolin-4-yl)-1-{[2-(trimethylsilyl)ethoxy]methyl}4,5,6,7-tetrahydro-1H-pyrazolo[4,3-c]pyridin-3-yl)propan-2-ol which was used in the next step without further purification.

Step 3

A mixture of 2-(5-(6,7-dimethoxyquinazolin-4-yl)-1-{[2-(trimethylsilyl)ethoxy] methyl}-4,5,6,7-tetrahydro-1H-pyrazolo[4,3-c]pyridin-3-yl)propan-2-ol (10 mg, 0.02 mmol, prepared as described above in Step 2), hydrogen chloride solution (0.25 mL, 36%) and tetrahydrofuran (0.80 mL) was heated at 60° C. for 30 minutes. The solvent was then evaporated and the resulting residue was purified by preparative HPLC to afford 2 mg (30%) of 2-[5-(6,7-dimethoxyquinazolin-4-yl)-4,5,6,7-tetrahydro-1H-pyrazolo[4,3-c]pyridin-3-yl]propan-2-ol hydroformate as a pale yellow solid. ¹H NMR (MeOD) δ (ppm) 8.50 (s, 1H), 8.30 (b, 1H), 7.34 (s, 1H), 7.18 (s, 1H), 5.00 (s, 2H) 4.07 (m, 2H), 4.01 (s, 3H), 4.00 (s, 3H), 3.08 (m, 2H), 1.54 (s, 6H), MS [M+H]=370.2, LC/MS (EI) t_{R} 3.98 min (Method I).

### Example 25

### 6,7-dimethoxy-4-(1-phenyl-1,4,5,7-tetrahydro-6H-pyrazolo[3,4-c]pyridin-6-yl)quinazoline

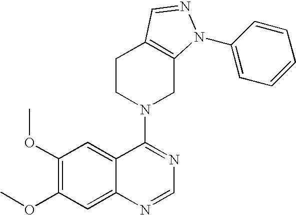

A mixture of 6,7-dimethoxy-4-(1,4,5,7-tetrahydro-6H-pyrazolo[3,4-c]pyridin-6-yl)quinazoline (50 mg, 0.2 mmol), phenylboronic acid (29 mg, 0.24 mmol), cupric acetate (29 mg, 0.16 mmol), triethylamine (110 μL, 0.80 mmol), pyridine (0.10 mL, 1.3 mmol), 1,4-dioxane (1.3 mL) and 4 Å molecular sieves (˜10 mg) was heated at 82° C. for 16 hours. Dichloromethane (30 mL) was then added and the organics were washed with 2% sodium bicarbonate solution (20 mL). The organics were concentrated and the residue was purified by preparative HPLC to afford 30 mg (50%) of 6,7-dimethoxy-4-(1-phenyl-1,4,5,7-tetrahydro-6H-pyrazolo[3,4-c]pyridin-6-yl)quinazoline as a pale yellow solid. [M+H]=388.1, LC/MS (EI) t_{R} 4.8 min (Method I). ¹H NMR (CDCl₃) δ (ppm) 8.69 (s, 1H), 7.79 (s, 1H), 7.66 (d, J=8.1 Hz, 2H), 7.57 (s, 1H), 7.45 (d, J=8.1 Hz, 2H), 7.30 (t, J=7.8 Hz, 1H), 7.20 (s, 1H), 4.94 (s, 2H), 4.14 (t, J=5.7 Hz, 2H), 4.07 (s, 3H), 4.04 (s, 3H), 3.28 (t, J=5.7 Hz, 2H). 8 mg (10%) of 6,7-dimethoxy-4-(2-phenyl-2,4,5,7-tetrahydro-6H-pyrazolo[3,4-c]pyridin-6-yl)quinazoline was also isolated. [M+H]=388.1, LC/MS (EI) t_{R} 4.71 min (Method I).

### Example 26

### d) 4-(6,7-dimethoxy-3,4-dihydroisoquinoline-2(1H)-yl)-6,7-dimethoxyquinazoline hydrochloride

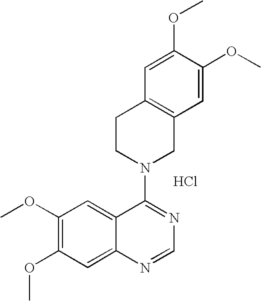

Crude 4-(6,7-dimethoxy-3,4-dihydroisoquinoline-2(1H)-yl)-6,7-dimethoxyquinazoline (600 mg, 1.57 mmol) was dissolved in dichloromethane (5 mL) and methanol (5 mL). 1.0 mL of 2.0 M hydrogen chloride in ether was added slowly to the solution with stirring, and after 5 minutes ethyl acetate (60 mL) was added. The resultant precipitate was collected by filtration, washed with ethyl acetate (15 mL) and dried to afford 540 mg (82%) of 4-(6,7-dimethoxy-3,4-dihydroisoquinoline-2(1H)-yl)-6,7-dimethoxyquinazoline hydrochloride. MS [M+H]=382.3, LC/MS (EI) t_{R} 5.09 min (Method B), ¹H NMR (DMSO) δ (ppm) 8.80 (s, 1H), 7.50 (s, 1H), 7.41 (s, 11H), 6.97 (s, 11H), 6.86 (s, 1H), 5.15 (s, 1H), 4.24 (m, 2H), 4.00 (s, 3H), 3.97 (s, 3H), 3.73 (s, 3H), 3.71 (s, 3H), 3.02 (m, 2H).

### Biological Examples

### Example 1

mPDE10A7 Enzyme Activity and Inhibition

Enzyme Activity:

To analyze the enzyme activity, 5 μL of serial diluted mPDE10A7 containing lysate were incubated with equal volumes of diluted (100-fold) fluorescein labeled cAMP or cGMP for 30 minutes in MDC HE 96-well assay plates at room temperature. Both the enzyme and the substrates were diluted in the following assay buffer: Tris/HCl (pH 8.0) 50 mM, MgCl₂ 5 mM, 2-mercaptoethanol 4 mM, BSA 0.33 mg/mL. After incubation, the reaction was stopped by adding 20 μL of diluted (400-fold) binding reagents and was incubated for an hour at room temperature. The plates were counted in an Analyst GT (Molecular Devices) for fluorescence polarization. An IMAP Assay kit (Molecular Device) was used to assess enzyme properties of mmPDE10A7. Data were analyzed with SoftMax Pro.

Enzyme Inhibition:

To check the inhibition profile, 10 μL of serial diluted compounds were incubated with 30 μl of diluted PDE enzymes in a 96-well polystyrene assay plate for 30 minutes at room temperature. After incubation, 5 μL of the compound-enzyme mixture were aliquoted into a MDC HE black plate, mixed with 5 μl of 100-fold diluted fluorescein labeled substrates (cAMP or cGMP), and incubated for 30 minutes at room temperature. The reaction was stopped by adding 20 μL of diluted binding reagents and counted in an Analyst GT for fluorescence polarization. The data were analyzed with SoftMax Pro. Certain chemical entities described herein showed inhibited mPDE10A7 in this assay typically with IC₅₀ values of less than 5 μM. For example, 2-(6,7-dimethoxyquinazolin-4-yl)-5-(2-methoxyethoxy)-3,4-dihydroisoquinoline-1(2H)-one exhibits an IC₅₀ of 218.96 nm.

### Example 2

Apomorphine Induced Deficits in Prepulse Inhibition of the Startle Response in Rats, an In Vivo Test for Antipsychotic Activity

The thought disorders that are characteristic of schizophrenia may result from an inability to filter, or gate, sensorimotor information. The ability to gate sensorimotor information can be tested in many animals as well as in humans. A test that is commonly used is the reversal of apomorphine-induced deficits in the prepulse inhibition of the startle response. The startle response is a reflex to a sudden intense stimulus such as a burst of noise. In this example, rats are exposed to a sudden burst of noise, at a level of 120 db for 40 msec, e.g. the reflex activity of the rats is measured. The reflex of the rats to the burst of noise may be attenuated by preceding the startle stimulus with a stimulus of lower intensity, at 3 to 12 db above background (65 db), which will attenuate the startle reflex by 20 to 80%.

The prepulse inhibition of the startle reflex, described above, may be attenuated by drugs that affect receptor signaling pathways in the CNS. One commonly used drug is the dopamine receptor agonist apomorphine. Administration of apomorphine will reduce the inhibition of the startle reflex produced by the prepulse. Antipsychotic drugs such as haloperidol will prevent apomorphine from reducing the prepulse inhibition of the startle reflex. This assay may be used to test the antipsychotic efficacy of PDE10 inhibitors, as they reduce the apomorphine-induced deficit in the prepulse inhibition of startle.

The preceding examples can be repeated with similar success by substituting the generically or specifically described reactants and/or operating conditions for those used in the preceding examples.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

1. At least one chemical entity chosen from compounds of Formulas (I) and (II): 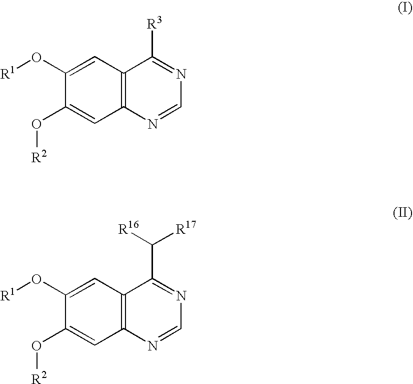
and individual stereoisomers, mixtures of stereoisomers, pharmaceutically acceptable solvates, and pharmaceutically acceptable salts thereof, wherein:
R¹ is chosen from H, alkyl having 1 to 4 carbon atoms, and alkyl having 1 to 4 carbon atoms substituted by at least one halogen;
R² is chosen from H, alkyl having 1 to 4 carbon atoms, and alkyl having 1 to 4 carbon atoms substituted by at least one halogen;
R³ is chosen from: 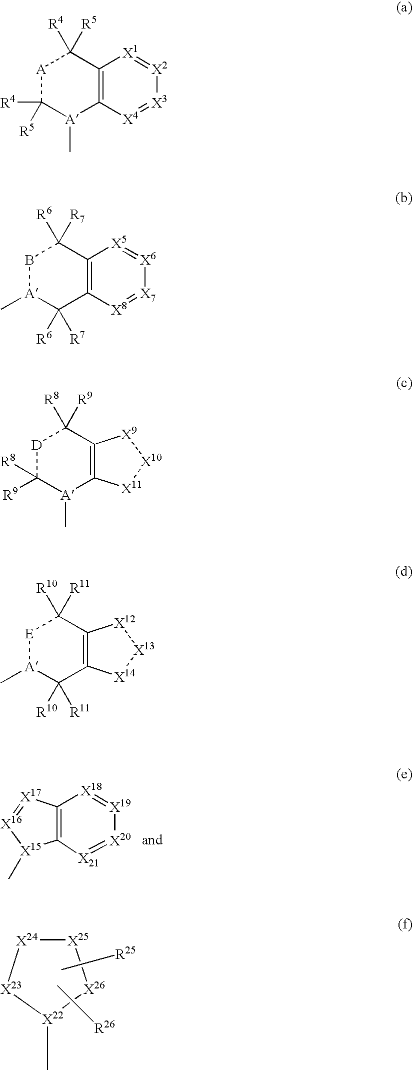
A′ is chosen from N and CH;
----A---- is chosen from a double bond, —CR⁴R⁵—, ═CR⁴—, —CR⁴═, —CR⁴R⁵—CR⁴R⁵—, ═CR⁴—CR⁴R⁵—, —CR⁴R⁵—CR⁴═, —CR⁴═CR⁵—, ═CR⁴—CR⁴═, —CR⁴R⁵—CR⁴R⁵—CR⁴R⁵—, ═CR⁴—CR⁴R⁵—CR⁴R⁵—, —CR⁴═CR⁴—CR⁴R⁵—, —CR⁴R⁵—CR⁴═CR⁴—, —CR⁴R⁵—CR⁴R⁵—CR⁴═, ═CR⁴—CR⁴═CR⁴—, —CR⁴═CR⁴—CR⁴═, and ═CR⁴—CR⁴R⁵—CR⁴═;
----B---- is chosen from a single bond, —CR⁶R⁷—, —CR⁶═, —CR⁶R⁷—CR⁶R⁷—, —CR⁶R⁷—CR⁶═, —CR⁶═CR⁷—, —CR⁶R⁷—CR⁶R⁷—CR⁶R⁷—, —CR⁶═CR⁶—CR⁶R⁷—CR⁶R⁷—CR⁶═CR⁶—, —CR⁶R⁷—CR⁶R⁷—CR⁶═, and —CR⁶═CR⁶—CR⁶═; —
----D---- is chosen from —CR⁸R⁹—, ═CR⁸—, —CR⁸═, —CR⁸R⁹—CR⁸R⁹—, ═CR⁸—CR⁸R⁹—, —CR⁸R⁹—CR⁸═, —CR⁸═CR⁹—, ═CR⁸—CR⁸═, —CR⁸R⁹—CR⁸R⁹—CR⁸R⁹—, ═CR⁸—CR⁸R⁹—CR⁸R⁹—, —CR⁸═CR⁸—CR⁸R⁹—, —CR⁸R⁹—CR⁸═CR⁸—, —CR⁸R⁹—CR⁸R⁹—CR⁸═, ═CR⁸—CR⁸═CR⁸—, —CR⁸═CR⁸—CR⁸═, and ═CR⁸—CR⁸R⁹—CR⁸—;
----E---- is chosen from —CR¹⁰R¹¹—, —CR¹⁰═, —CR¹⁰R¹¹—CR¹⁰R¹¹—, —CR¹⁰R¹¹—CR¹⁰═, —CR¹⁰═CR¹¹—, —CR¹⁰R¹¹—CR¹⁰R —CR¹⁰R —, —CR¹⁰═CR¹⁰—CR¹⁰R¹¹—, —CR¹⁰R¹¹—CR¹⁰═CR¹⁰—, —CR¹⁰R¹¹—CR¹⁰R¹¹—CR¹⁰═, and —CR¹⁰═CR¹⁰—CR¹⁰—;
the dotted lines in the 5-membered ring of formula (c) independently represent a single bond or a double bond; with the proviso there is at least one double bond between X⁹ and X¹⁰ or X¹⁰ and X¹¹;
the dotted lines in the 5-membered ring of formula (d) independently represent a single bond or a double bond; with the proviso there is at least one double bond between X¹² and X¹³ or X¹³ and X¹⁴;
the dotted lines in formula (f) independently represent a single bond or a double bond, with the proviso that when two double bonds are present, they are not adjacent to each other;
X¹, X², X³, X⁴, X⁵, X⁶, X⁷, X⁸, X¹⁸, X¹⁹, X²⁰ and X²¹ are each independently chosen from N and CR¹², and wherein two adjacent X¹-X⁴, X⁵-X⁸, and X¹⁸-X²¹ groups can each be CR¹² in which the two R¹² groups taken together form a fused ring structure chosen from methylenedioxy, ethylenedioxy group, difluoromethylenedioxy, and tetrafluoroethylenedioxy;
X⁹, X¹⁰, X¹¹, X¹², X¹³, and X¹⁴ are each independently chosen from S, O, N, NR¹², C(R¹²)₂, and CR¹²;
X¹⁵, X¹⁶ and X¹⁷ are each independently chosen from N and CR¹² wherein at least two of X¹⁵, X¹⁶ and X¹⁷ are not CR¹²;
X²² is chosen from N, C and CR¹² and X²³, X²⁴, X²⁵, and X²⁶ are each independently chosen from O, S, N, NR¹², C, CHR¹², C(R¹²)₂, and CR¹²;
wherein at least two of X²², X²³, X²⁴, X²⁵, and X²⁶ are not chosen from C, CHR¹² and CR¹²;
R⁴, R⁵, R⁶, R⁷, R⁸, R⁹, R¹⁰, and R¹¹ are independently chosen from
absent,
H,
carboxy,
alkyl having 1 to 8,
substituted alkyl having 1 to 8 carbon atoms and substituted with at least one group chosen from halogen, C₁₋₄-alkyl, C₁₋₄-alkoxy, and oxo,
cycloalkyl having 3 to 12 carbon atoms,
substituted cycloalkyl having 3 to 12 carbon atoms and substituted with at least one group chosen from halogen, C₁₋₄-alkyl, C₁₋₄-alkoxy, and oxo,
cycloalkylalkyl having 4 to 12 carbon atoms, and
substituted cycloalkylalkyl having 4 to 12 carbon atoms and substituted with at least one group chosen from halogen, C₁₋₄-alkyl, C₁₋₄-alkoxy, and oxo, or
R⁴ and R⁵ together form a cycloalkyl group chosen from 3 to 8 membered spiro cycloalkyl and 3 to 8 membered fused cycloalkyl, or
R⁶ and R⁷ together form a cycloalkyl group chosen from 3 to 8 membered spiro cycloalkyl and 3 to 8 membered fused cycloalkyl, or
R⁸ and R⁹ together form a cycloalkyl group chosen from 3 to 8 membered spiro cycloalkyl and 3 to 8 membered fused cycloalkyl, or
R¹⁰ and R¹¹ together form a cycloalkyl group chosen from 3 to 8 membered spiro cycloalkyl and 3 to 8 membered fused cycloalkyl, or
one or more of R⁴ and R⁵ and the carbon atom to which they are attached form a C(═O) group, or
one or more of R⁶ and R⁷ and the carbon atom to which they are attached form a C(═O) group, or
one or more of R⁸ and R⁹ and the carbon atom to which they are attached form a C(═O) group, or
one or more of R¹⁰ and R¹¹ and the carbon atom to which they are attached, in each case form a C(═O) group,
R¹² is chosen from
H,
alkyl having up to 12 carbon atoms,
substituted alkyl having up to 12 carbon atoms and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from —CH═CH— and —C≡C—;
cycloalkyl having 3 to 12 carbon atoms,
substituted cycloalkyl having 3 to 12 carbon atoms and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from —CH═CH— and —C≡C—;
cycloalkylalkyl having up to 12 carbon atoms,
substituted cycloalkylalkyl having up to 12 carbon atoms and substituted and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR³, —SCONHR¹³, —SCSNHR¹³, and —NHCSNHR¹³ and wherein optionally one or more —CH₂CH₂— groups is replaced in each case by a group chosen from —CH═CH— and —C≡C—;
heterocyclyl,
heterocyclyl substituted with at least one group chosen from halogen, C₆₋₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
heteroaryl,
heteroaryl substituted with at least one group chosen from halogen, C₆₋₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
aryl having 6 to 14 carbon atoms,
substituted aryl having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
arylalkyl having 7 to 16 carbon atoms,
substituted arylalkyl having 7 to 16 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
heteroarylalkyl wherein the heteroaryl portion has 5 to 10 ring atoms in which at least 1 ring atom is a heteroatom and the alkyl portion has 1 to 3 carbon atoms,
substituted heteroarylalkyl wherein the heteroaryl portion has 5 to 10 ring atoms in which at least 1 ring atom is a heteroatom and the alkyl portion has 1 to 3 carbon atoms and wherein the heteroaryl portion is substituted by at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
aryloxy having 6 to 14 carbon atoms,
substituted aryloxy having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy;
heteroaryloxy having 5 to 10 ring atoms in which at least 1 ring atom is a heteroatom,
substituted heteroaryloxy having 5 to 10 ring atoms in which at least 1 ring atom is a heteroatom, and substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
halogen, hydroxy, C₁₋₄-alkoxy, C₁₋₄-alkoxy-C₁₋₄-alkoxy, C₄₋₁₂-cycloalkylalkyloxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, —C₁₋₄-alkylthio, C₁₋₄-alkylsulphinyl, C₁₋₄-alkylsulphonyl, —SO₂NHR¹⁹, —SO₂NR¹⁸R¹⁹, —SO₂R²⁰, —NHSO₂R¹³, —NR¹³COR¹³, —CONHR¹³, —CONR¹³R¹⁹, —NHCONHR¹³, —OCONHR¹³, —NHCOOR¹³, —SCONHR¹³, —SCSNHR¹³, and —NHCSNHR¹³;
R¹³ is chosen from
H,
alkyl having 1 to 8 carbon atoms,
substituted alkyl having 1 to 8 carbon atoms and substituted with at least one group chosen from halogen, C₁₋₄-alkyl, C₁₋₄-alkoxy, and oxo,
cycloalkyl having 3 to 12 carbon atoms,
substituted cycloalkyl having 3 to 12 carbon atoms and substituted with at least one group chosen from halogen, C₁₋₄-alkyl, C₁₋₄-alkoxy, and oxo;
cycloalkylalkyl having 4 to 12 carbon atoms, and
substituted cycloalkylalkyl having 4 to 12 carbon atoms and substituted with at least one group chosen from halogen, C₁₋₄-alkyl, C₁₋₄-alkoxy, and oxo;
R¹⁶ is chosen from
aryl having 6 to 14 carbon atoms,
substituted aryl having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
heteroaryl having 5 to 10 ring atoms in which at least 1 ring atom is a heteroatom,
substituted heteroaryl having 5 to 10 ring atoms in which at least 1 ring atom is a heteroatom and substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
heterocyclyl,
substituted heterocyclyl substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
carbocyclic, and
substituted carbocyclic substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy;
R¹⁷ is chosen from
alkyl having 1 to 12 carbon atoms,
substituted alkyl having 1 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸ wherein optionally one or more —CH₂— groups is, in each case independently, replaced by —O—, —S—, or —NH— and wherein optionally one or more —CH₂CH₂— groups is replaced in each case by —CH═CH— or —C≡C—,
cycloalkyl having 3 to 12 carbon atoms,
substituted cycloalkyl having 3 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄ alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸, wherein optionally one or more —CH₂— groups is, in each case independently, replaced by —O—, —S—, or —NH— and wherein optionally one or more —CH₂CH₂— groups is replaced in each case by —CH═CH— or —C≡C—,
cycloalkylalkyl,
substituted cycloalkylalkyl substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸ wherein optionally one or more —CH₂— groups is, in each case independently, replaced by —O—, —S—, or —NH— and wherein optionally one or more —CH₂CH₂— groups is replaced in each case by —CH═CH— or —C≡C—,
halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸CR¹⁸, —CONHR¹⁸, —NHCONHR¹⁸, —OCONHR¹⁸, —NHCOOR¹⁸, —SCONHR¹⁸, —SCSNHR¹⁸, and NHCSNHR¹⁸;
R¹⁸ is chosen from
H,
alkyl having 1 to 8 carbon atoms, and substituted alkyl having 1 to 8 carbon atoms substituted with at least one group chosen from halogen, C₁₋₄-alkyl, C₁₋₄-alkoxy, and oxo;
R¹⁹ is chosen from
H,
alkyl having 1 to 8 carbon atoms,
substituted alkyl having 1 to 8 carbon atoms and substituted with at least one group chosen from halogen, C₁₋₄-alkyl, C₁₋₄-alkoxy, and oxo,
cycloalkyl having 3 to 10 carbon atoms,
substituted cycloalkyl having 3 to 10 carbon atoms and substituted with at least one group chosen from halogen, C₁₋₄-alkyl, C₁₋₄-alkoxy, and oxo,
cycloalkylalkyl having 4 to 12 carbon atoms,
substituted cycloalkylalkyl having 4 to 12 carbon atoms and substituted with at least one group chosen from halogen, C₁₋₄-alkyl, C₁₋₄-alkoxy, and oxo,
heteroaryl,
heteroaryl substituted with at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
heterocyclyl, and
heterocyclyl substituted with at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
R²⁰ is chosen from
heterocyclyl, and
heterocyclyl substituted by at least one group chosen from halogen, C₆₋₁₄-aryl-C₁₋₄-alkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
R²⁵ and R²⁶ are independently chosen from
H,
carboxy,
alkyl having 1 to 8 carbon atoms,
substituted alkyl having 1 to 8 carbon atoms and substituted with at least one group chosen from halogen, C₁₋₄-alkyl, C₁₋₄-alkoxy, and oxo,
cycloalkyl having 3 to 12 carbon atoms,
substituted cycloalkyl having 3 to 12 carbon atoms and substituted with at least one group chosen from halogen, C₁₋₄-alkyl, C₁₋₄-alkoxy, and oxo,
cycloalkylalkyl having 4 to 12 carbon atoms,
cycloalkylalkyl having 4 to 12 carbon atoms substituted with at least one group chosen from halogen, C₁₋₄-alkyl, C₁₋₄-alkoxy, and oxo, or
R²⁵ and R²⁶ together form a cycloalkyl group, spiro or fused, having 3 to 8 carbon atoms, or
R²⁵ and R²⁶ and the carbon atom to which they are attached form a C(═O) group; with the proviso that
said compound of Formulas (I) and (II) is not chosen from
6,7-dimethoxy-4-(2-methyl-3,4-dihydroquinolin-1(2H)-yl)quinazoline;
4-(7-bromo-3,4-dihydroquinolin-1(2H)-yl)-6,7-dimethoxyquinazoline;
4-(5-bromo-3,4-dihydroquinolin-1(2H)-yl)-6,7-dimethoxyquinazoline;
6,7-dimethoxy-4-[7-(trifluoromethyl)-3,4-dihydroquinolin-1(2H)-yl]quinazoline;
6,7-dimethoxy-4-(6-methyl-3,4-dihydroquinolin-1(2H)-yl)quinazoline;
4-(3,4-dihydroquinolin-1(2H)-yl)-6,7-dimethoxyquinazoline;
8-bromo-1-(6,7-dimethoxyquinazolin-4-yl)-2,3,4,5-tetrahydro-1H-1-benzazepine;
9-bromo-1-(6,7-dimethoxyquinazolin-4-yl)-2,3,4,5-tetrahydro-1H-1-benzazepine;
1-(6,7-dimethoxyquinazolin-4-yl)-2,3,4,5-tetrahydro-1H-1-benzazepine;
1-(6,7-dimethoxyquinazolin-4-yl)-1H-indole-3-carbaldehyde; 4-(1H-indol-1-yl)-6,7-dimethoxyquinazoline;
4-(1-H-benzotriazol-1-yl)-6,7-dimethoxyquinazoline;
4-(1-H-benzimidazol-1-yl)-6,7-dimethoxyquinazoline;
4-(1-H-indazol-1-yl)-6,7-dimethoxyquinazoline;
4-(5-fluorophenyl)-2-[4-(methylsulfonyl)phenyl)-1H-imidazol-4-yl)-6,7-dimethoxyquinazoline;
4-(1-cyclopropylmethyl)-4-(4-fluorophenyl)-1H-imidazol-5-yl)-6,7-dimethoxyquinazoline;
4-(5-(4-fluorophenyl)-3-phenyl-1H-1,2,4-triazol-1-yl)-6,7-dimethoxyquinazoline;
1-(6,7-dimethoxy-4quinazolinyl)-1H-pyrazole-3-amine;
N-[2-(6,7-dimethoxy-quinazolin-4-yl)-1,2,3,4-tetrahydro-isoquinolin-7-yl]-2,2-dimethyl-propionamide;
N-[2-(6,7-dimethoxy-quinazoline-4-yl)-1,2,3,4-tetrahydro-isoquinolin-7-yl]-acetamide;
6,7-dimethoxy-4-[8-(morpholine-4-sulfonyl)-3,4-dihydro-1H-isoquinolin-2-yl]-quinazoline;
6,7-dimethoxy-4-[8-(4-methyl-piperazine-1-sulfonyl)-3,4-dihydro-1H-isoquinolin-2-yl]-quinazoline;
4-(7,8-dimethoxy-3,4-dihydro-1H-isoquinolin-2-yl)-6-ethoxy-7-methoxy-quinazoline;
4-(6,7-dimethoxy-3,4-dihydro-1H-isoquinolin-2-yl)-6-ethoxy-7-methoxy-quinazoline;
4-(6,7-dimethoxy-3-methyl-3,4-dihydro-1H-isoquinolin-2-yl)-6-ethoxy-7-methoxy-quinazoline;
4-(3,4-dihydro-1H-isoquinolin-2-yl)-6-ethoxy-7-methoxy-quinazoline;
2-(6,7-dimethoxy-quinazolin-4-yl)-1,2,3,4-tetrahydro-isoquinolin-7-yl-amine; and
6,7-dimethoxy-4-(3-propyl-3,4-dihydro-1H-isoquinolin-2-yl)-quinazoline.

2. At least one chemical entity of claim 1 wherein the at least one chemical entity is chosen from compounds of Formula (I).

3. At least one chemical entity of claim 1 wherein A′ is —N—.

4. At least one chemical entity of claim 1 wherein R³ is

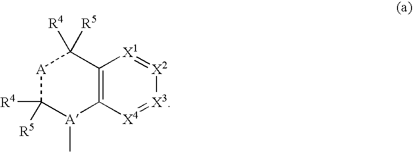

5. At least one chemical entity of claim 4 wherein A′ is —N—, ----A---- is —CR⁴R⁵— and X¹-X⁴ are CH, and R⁴ and R⁵ are not all H, and if one of the R⁴ and R⁵ groups is methyl then at least one of the remaining R⁴ and R⁵ groups is other than H.

6. At least one chemical entity of claim 4 wherein A′ is —N—, ----A---- is —CH₂—, when all R⁴ and R⁵ are H, then at least one of X¹-X⁴ is CR¹² in which R¹² is not chosen from H, halogen, alkyl, and haloalkyl.

7. At least one chemical entity of claim 4 wherein A′ is —N—, ----A---- is —CH₂—, all R⁴ and R⁵ are H, and at least one of X¹-X⁴ is CR¹² in which R¹² is chosen from hydroxy, C₁₋₄-alkoxy, C₁₋₄-alkoxy-C₁₋₄-alkoxy, C₄₋₁₂-cycloalkylalkyloxy, aryloxy, halogenated C₁₋₄ alkoxy, and C₂₋₄-hydroxyalkoxy.

8. At least one chemical entity of claim 4 wherein ----A---- is —CR⁴R⁵—, and each of the R⁴ and R⁵ groups is absent, H, alkyl, COOH, or one set of R⁴ and R⁵ together with the carbon to which they are attached form a C(═O) group.

9. At least one chemical entity of claim 4 wherein A′ is —N—, ----A---- is —CR⁴R⁵—CR⁴R⁵, and when all R⁴ and R⁵ are H, then at least one of X¹-X⁴ is CR¹² in which R¹² is not chosen from H, alkyl, and halogen.

10. At least one chemical entity of claim 4 wherein A′ is —N—, ----A---- -is —CR⁴R⁵—CR⁴R⁵, and when all R⁴ and R⁵ are H, then at least one of X¹-X⁴ is CR¹² in which R¹² is not chosen from H, CH₃, and halogen.

11. (canceled)

12. At least one chemical entity of claim 4 wherein A′ is —N—, ----A---- is a double bond, and when all R⁴ and R⁵ are H or are absent, then at least one of X¹-X⁴ is CR¹² in which R¹² is not chosen from H and CHO.

13. At least one chemical entity of claim 4 wherein A′ is —N—, ----A---- is a double bond, and when all R⁴ and R⁵ are H or are absent, then at least one of X¹-X⁴ is CR¹² in which R¹² is not chosen from H and COR¹³.

14. (canceled)

15. At least one chemical entity of claim 4 wherein X¹-X⁴ are each CR¹², where R¹² is chosen from H and alkyl, A′ is —N—, and ----A---- is a double bond, and R⁴ and R⁵ are other than COR¹³.

16. At least one chemical entity of claim 4 wherein X¹-X⁴ are each CR¹³, A′ is —N—, and ----A---- is a double bond, then and at least one R¹² is not chosen from H, halogen, CN, C₁₋₄ alkyl, nitro, NH₂, NH(C₁₋₄ alkyl), N(C₁₋₄ alkyl)₂, COOH, COO(C₁₋₄ alkyl), CHO, CONH₂, CONH(C₁₋₄ alkyl), CON(C₁₋₄ alkyl)₂, O(C₁₋₄ alkyl), phenoxy, and CH(OC₁₋₄ alkyl)₂.

17. At least one chemical entity of claim 4 wherein X¹-X⁴ are each CH or CCH₃, A′ is —N—, and ----A---- is a double bond, and R⁴ and R⁵ are other than CHO.

18. (canceled)

19. At least one chemical entity of claim 4 wherein X¹-X⁴ are each CH, A′ is —N—, and ----A---- is a double bond, and R⁴ and R⁵ are other than CHO.

20. (canceled)

21. At least one chemical entity of claim 4 wherein one set of R⁴ and R⁵ together with the carbon to which they are attached form a C(═O) group.

22. At least one chemical entity of claim 1 wherein ----A---- represents a double bond or —CR⁴R⁵—.

23. At least one chemical entity of claim 1 wherein ----A---- represents a double bond or —CR⁴R⁵— and A′ is —N—.

24. At least one chemical entity of claim 1 wherein R³ is

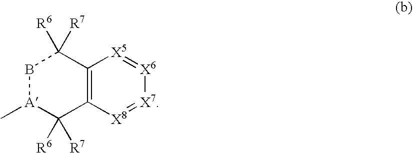

25. At least one chemical entity of claim 24 wherein one set of R⁶ and R⁷ together with the carbon to which they are attached form a C(═O) group.

26. At least one chemical entity of claim 24 wherein ----B---- represents a single bond or —CR⁵R⁶—.

27. At least one chemical entity of claim 24 wherein ----B---- represents a single bond or —CR⁵R⁶— and A′ is —N—.

28. At least one chemical entity of claim 24 wherein R³ is a group of the formula (b1), 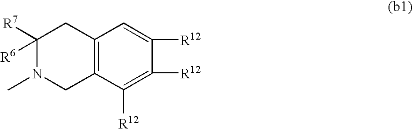
where R⁶ and R⁷ are each not alkyl, and the R¹² group attached to the 8-position of the isoquinoline is not chosen from alkoxy and —SO₂R²⁰ in which R²⁰ is chosen from morpholino, substituted morpholino, piperazino, and substituted piperazino, the R¹² group attached to the 7-position of the isoquinoline is not chosen from alkoxy, amino, alkylamino, and —NR¹³COR¹³ in which R¹³ in each case is chosen from H and alkyl, the R¹² group attached to the 6-position of the isoquinoline is not alkoxy, and R⁶, R⁷, and the three R¹² are not all H.

29-31. (canceled)

32. At least one chemical entity of claim 24 wherein R³ is a group of the following formula (b2), 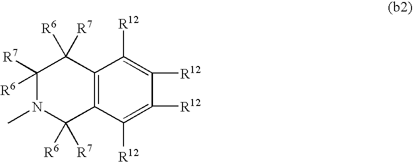
at least one R¹² is not chosen from H, alkoxy, amino, alkylamino, —COR¹³, —COOR¹³, —SO₂NHR¹³, —SO₂NHR¹⁹, —SO₂NR¹³¹⁸R¹⁹, —SO₂R²⁰, —NHSO₂R¹³, —NR¹³COR¹³, —CONHR¹³, —CONR¹³R¹⁹, CONH-cycloalkyl, —NHCONHR¹³, and —NHCOOR¹³, and at least two R¹² are not alkoxy, and the R⁶, R⁷, and R¹² groups are not all H.

33. At least one chemical entity of claim 24 wherein R³ is a group chosen from 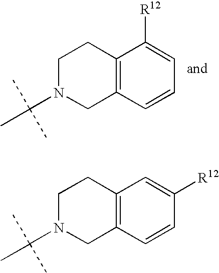
where the ring can optionally be further substituted with R¹².

34. At least one chemical entity of claim 33 wherein R¹² is substituted heteroaryl.

35. At least one chemical entity of claim 33 wherein R¹² is chosen from optionally substituted saturated heterocyclyl and optionally substituted partially saturated heterocyclyl.

36. At least one chemical entity of claim 35 wherein R¹² is chosen from optionally substituted piperazinyl, optionally substituted piperidinyl, and optionally substituted morpholinyl.

37. At least one chemical entity of claim 24 wherein R³ is chosen from:

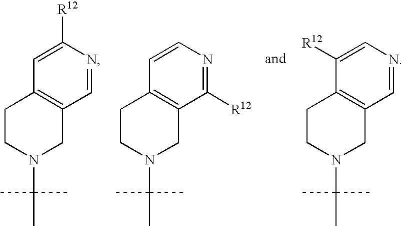

38. At least one chemical entity of claim 37 wherein R¹² is chosen from alkyl, C₁₋₄alkoxy, halogenated C₁₋₄alkoxy, cycloalkyl, aryl, heteroaryl, heterocyclyl, C₁₋₄ alkylamino, di-C₁₋₄-alkylamino, —COR¹³, C₁₋₄-alkylthio, C₁₋₄-alkylsulphinyl, C₁₋₄-alkylsulphonyl, —NR¹³COR¹³, —CONHR¹³, —CONR¹³R¹⁹, —NHSO₂R¹³, —SO₂NHR¹⁹, and —SO₂NR¹⁸R¹⁹ and wherein the ring in R¹² is optionally substituted.

39. At least one chemical entity of claim 38 wherein R¹² is chosen from cycloalkyl, aryl, heteroaryl, and heterocyclyl, each of which is optionally substituted.

40. At least one chemical entity of claim 24 wherein R³ is a group of formula:

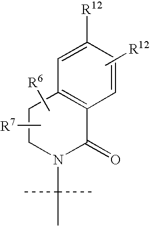

41. At least one chemical entity of claim 40 wherein R³ is chosen from: 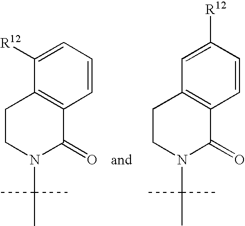
optionally further substituted with R¹².

42. At least one chemical entity of claim 41 wherein R¹² is optionally substituted heteroaryl.

43. At least one chemical entity of claim 41 wherein R¹² is a heterocyclyl group chosen from optionally substituted saturated heterocyclyl and optionally substituted partially saturated heterocyclyl groups.

44. At least one chemical entity of claim 43 wherein R¹² is chosen from optionally substituted piperazinyl, optionally substituted piperidinyl, and optionally substituted morpholinyl.

45. At least one chemical entity of claim 1 wherein R⁴, R⁵, R⁶, and R⁷ are independently chosen from absent, H, carboxy, and CH₃.

46. At least one chemical entity of claim 1 wherein R³ is

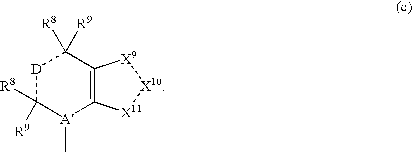

47. At least one chemical entity of claim 1 wherein R³ is

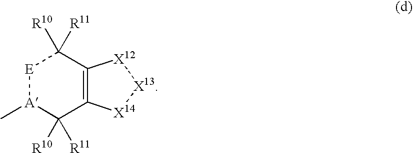

48. At least one chemical entity of claim 1 wherein R³ is chosen from: 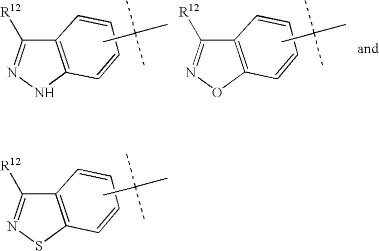
where: R¹² is chosen from cycloalkyl, cycloalkylalkyl, arylalkyl, heteroarylalkyl, aryl, heteroaryl, heterocyclyl, C₁₋₄ alkylamino, di-C₁₋₄-alkylamino, —COR¹³, C₁₋₄-alkylthio, C₁₋₄-alkylsulphinyl, C₁₋₄-alkylsulphonyl, —NR¹³COR¹³, —CONHR¹³, —CONR¹³R¹⁹, —NHSO₂R³, —SO₂NHR¹⁹, and —SO₂NR¹⁸R¹⁹, each of which is optionally substituted.

49-50. (canceled)

51. At least one chemical entity of claim 1 wherein R³ is

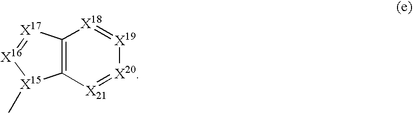

52. At least one chemical entity of claim 51 wherein (i) X¹⁵-X¹⁷ are each N, or (ii) when X¹⁵ and X¹⁷ are N and X¹⁶ is CH, or (iii) when X¹⁵ and X¹⁶ are N and X¹⁷ is CH, then at least one of X¹⁸, X¹⁹, X²⁰, or X²¹ is other than CH.

53. At least one chemical entity of claim 51 wherein R³ is chosen from

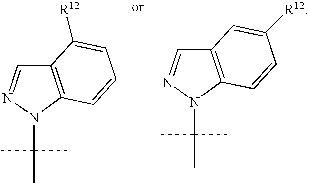

54. At least one chemical entity of claim 53 wherein R¹² is chosen from cycloalkyl, cycloalkylalkyl, arylalkyl, heteroarylalkyl, aryl, heteroaryl, heterocyclyl, C₁₋₄ alkylamino, di-C₁₋₄-alkylamino, —COR¹³, C₁₋₄alkylthio, C₁₋₄-alkylsulphinyl, C₁₋₄-alkylsulphonyl, —NR¹³COR¹³, —CONHR¹³, —CONR¹³R¹⁹, —NHSO₂R¹³ SO₂NHR¹⁹, and —SO₂NR¹⁸R¹⁹, each of which is optionally substituted.

55. At least one chemical entity of claim 54 wherein R¹² is chosen from phenyl and heterocyclyl, each of which is optionally substituted.

56. At least one chemical entity of claim 51 wherein R³ is: 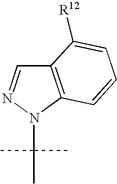
where R¹² is chosen from phenyl, heteroaryl, a five-membered heterocyclyl group which is chosen from saturated and partially saturated five-membered heterocyclyl groups, and a six-membered heterocyclyl group which is chosen from saturated and partially saturated six-membered heterocyclyl groups, each of which is optionally substituted.

57. At least one chemical entity of claim 56 wherein R¹² is chosen from morpholin-4-yl, piperazin-1-yl, and pyridinyl, each of which is optionally substituted.

58-60. (canceled)

61. At least one chemical entity of claim 1 wherein R³ is

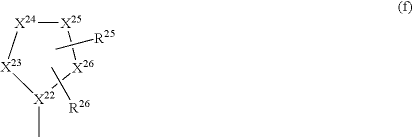

62. At least one chemical entity of claim 61 wherein at least one of X²²-X²⁶ is CR¹² and at least one R¹² is not chosen from amino, cycloalkylalkyl, substituted phenyl, and phenyl.

63. At least one chemical entity of claim 61 wherein two of X²²-X²⁵ are independently chosen from N and NR¹² and the rest of X²²-X²⁵ are independently chosen from C and CR¹².

64. At least one chemical entity of claim 61 wherein at least one of X²²-X²⁶ is CR¹² and at least one R¹² is not chosen from amino, C₁₋₄ alkylamino, di-C₁₋₄-alkylamino, cycloalkylalkyl, substituted phenyl, and phenyl.

65-68. (canceled)

69. At least one chemical entity of claim 61 wherein R³ is a group of formula:

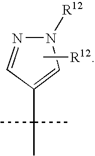

70. At least one chemical entity of claim 69 wherein one R¹² is chosen from hydrogen and alkyl and the other is chosen from aryl, heteroaryl, arylalkyl, heteroarylalkyl, and heterocyclyl, each of which is optionally substituted.

71-77. (canceled)

78. At least one chemical entity of claim 1 wherein R¹ and R² are alkyl.

79-82. (canceled)

83. At least one chemical entity of claim 1 wherein the compound of Formulas (I) and (II) is chosen from:
4-(6,7-dimethoxy-3,4-dihydroisoquinoline-2(1H)-yl)-6,7-dimethoxyquinazoline,
4-(6,7-Dimethoxy-1-methyl-3,4-dihydroisoquinoline-2(1H)-yl)-6,7-dimethoxyquinazoline,
4-(1-isopropyl-6,7-dimethoxy-3,4-dihydroisoquinoline-2(1H)-yl)-6,7-dimethoxyquinazoline,
4-(6,7-dimethoxy-3,4-dihydroisoquinoline-2(1H)-yl)-6,7-dimethoxyquinazoline hydrochloride,
(6,7-dimethoxyquinazolin-4-yl)(1-isopropyl-4,4-dimethyl-4,5-dihydro-1H-imidazol-2-yl)acetonitrile,
4-(6,7-dimethoxy-3-methyl-3,4-dihydroisoquinoline-2(1H)-yl)-6,7-dimethoxyquinazoline,
4-(1,3-dihydro-2H-isoindol-2-yl)-6,7-dimethoxyquinazoline,
(3S)-2-(6,7-dimethoxyquinazolin-4-yl)-6,7-dimethoxy-1,2,3,4-tetrahydroisoquinoline-3-carboxylic acid,
4-(5-bromo-1H-indazol-1-yl)-6,7-dimethoxyquinazoline,
4-(5-bromo-3H-indazol-3-yl)-6,7-dimethoxyquinazoline,
2-(6,7-dimethoxyquinazolin-4-yl)-1,2,3,4-tetrahydroisoquinoline-6,7-diol,
4-(4,7-dihydrothieno[2,3-c]pyridin-6(5H)-yl)-6,7-dimethoxyquinazoline,
4-[(3S)-6,7-dimethoxy-3-methyl-3,4-dihydroisoquinoline-2(1H)-yl]-6,7-dimethoxyquinazoline,
4-[(3R)-6,7-dimethoxy-3-methyl-3,4-dihydroisoquinoline-2(1H)-yl]-6,7-dimethoxyquinazoline,
6,7-dimethoxy-4-[5-(2-methoxyethoxy)-3,4-dihydroisoquinoline-2(1H)-yl]quinazoline,
6,7-dimethoxy-4-[7-(2-methoxyethoxy)-3,4-dihydroisoquinoline-2(1H)-yl]quinazoline,
6,7-dimethoxy-4-[6-(2-methoxyethoxy)-3,4-dihydroisoquinoline-2(1H)-yl]quinazoline,
2-(6,7-dimethoxyquinazolin-4-yl)-6,7-dimethoxy-3,4-dihydroisoquinoline-1(2H)-one,
2-(6,7-dimethoxyquinazolin-4-yl)-5-(2-methoxyethoxy)-3,4-dihydroisoquinoline-1(2H)-one,
1-benzyl-3-(6,7-dimethoxyquinazolin-4-yl)imidazolidin-4-one,
4-(1-benzyl-1H-pyrazol-4-yl)-6,7-dimethoxyquinazoline hydroformate,
[2-(6,7-dimethoxyquinazolin-4-yl)-1,2,3,4-tetrahydroisoquinolin-6-yl]methanol,
Ethyl 5-(6,7-dimethoxyquinazolin-4-yl)-4,5,6,7-tetrahydro-1H-pyrazolo[4,3-c]pyridine-3-carboxylate,
5-(6,7-dimethoxyquinazolin-4-yl)-4,5,6,7-tetrahydro-1H-pyrazolo[4,3-c]pyridine-3-carboxylic acid trifluoroacetate,
N-cyclopropyl-5-(6,7-dimethoxyquinazolin-4-yl)-4,5,6,7-tetrahydro-1H-pyrazolo[4,3-c]pyridine-3-carboxamide hydroformate,
N-(cyclopropylmethyl)-5-(6,7-dimethoxyquinazolin-4-yl)-4,5,6,7-tetrahydro-1H-pyrazolo[4,3-c]pyridine-3-carboxamide hydroformate;
2-(6,7-dimethoxyquinazolin-4-yl)-1,2,3,4-tetrahydroisoquinoline-6-carboxylic acid;
6,7-dimethoxy-4-(6-(methoxymethyl)-3,4-dihydroisoquinoline-2(1H)-yl)quinazoline;
4-(6,7-dihydro-1H-pyrazolo[4,3-c]pyridin-5(4H)-yl)-6,7-dimethoxyquinazoline formate;
2-(2-(6,7-dimethoxyquinazolin-4-yl)-1,2,3,4-tetrahydroisoquinolin-5-yloxy)ethanol;
2-(6,7-dimethoxyquinazolin-4-yl)-1,2,3,4-tetrahydroisoquinoline-6-carboxylic acid trifluoroacetate;
N-(cyclopropylmethyl)-2-(6,7-dimethoxyquinazolin-4-yl)-1,2,3,4-tetrahydroisoquinoline-6-carboxamide formate;
N-cyclopropyl-2-(6,7-dimethoxyquinazolin-4-yl)-1,2,3,4-tetrahydroisoquinoline-6-carboxamide formate;
N-cyclopropyl-4-(6,7-dimethoxy-3,4-dihydroisoquinoline-2(1H)-yl)-6,7-dimethoxyquinazolin-2-amine formate;
2-(2-(6,7-dimethoxyquinazolin-4-yl)-1,2,3,4-tetrahydroisoquinolin-7-yloxy)ethanol;
1-(5-(6,7-dimethoxyquinazolin-4-yl)-4,5,6,7-tetrahydropyrazolo[4,3-c]pyridin-1-yl)-3-methylbutane-1-one formate;
2-(5-(6,7-dimethoxyquinazolin-4-yl)-4,5,6,7-tetrahydro-1H-pyrazolo[4,3-c]pyridin-3-yl)propan-2-ol formate;
6,7-dimethoxy-4-(3-(prop-1-en-2-yl)-6,7-dihydro-1H-pyrazolo[4,3-c]pyridin-5(4H)-yl)quinazoline formate;
5-(6,7-dimethoxyquinazolin-4-yl)-N-methyl-4,5,6,7-tetrahydro-1H-pyrazolo[4,3-c]pyridine-3-carboxamide formate;
5-(6,7-dimethoxyquinazolin-4-yl)-4,5,6,7-tetrahydro-1H-pyrazolo[4,3-c]pyridine-3-carboxamide;
(5-(6,7-dimethoxyquinazolin-4-yl)-4,5,6,7-tetrahydro-1H-pyrazolo[4,3-c]pyridin-3-yl)methanol;
2-(5-(6,7-dimethoxyquinazolin-4-yl)-4,5,6,7-tetrahydro-1H-pyrazolo[4,3-c]pyridin-3-yl)propan-2-ol;
4-(4,5-dihydro-1H-pyrazolo[3,4-c]pyridin-6(7H)-yl)-6,7-dimethoxyquinazoline;
6,7-dimethoxy-4-(1-methyl-4,5-dihydro-1H-pyrazolo[3,4-c]pyridin-6(7H)-yl)quinazoline;
4-(1-ethyl-4,5-dihydro-1H-pyrazolo[3,4-c]pyridin-6(7H)-yl)-6,7-dimethoxyquinazoline;
4-(1-benzyl-4,5-dihydro-1H-pyrazolo[3,4-c]pyridin-6(7H)-yl)-6,7-dimethoxyquinazoline;
6,7-dimethoxy-4-(1-phenyl-4,5-dihydro-1H-pyrazolo[3,4-c]pyridin-6(7H)-yl)quinazoline;
4-(1,3-dimethyl-4,5-dihydro-1H-pyrazolo[3,4-c]pyridin-6(7H)-yl)-6,7-dimethoxyquinazoline;
ethyl 5-(6,7-dimethoxyquinazolin-4-yl)-1-ethyl-4,5,6,7-tetrahydro-1H-pyrazolo[4,3-c]pyridine-3-carboxylate;
(5-(6,7-dimethoxyquinazolin-4-yl)-1-methyl-4,5,6,7-tetrahydro-1H-pyrazolo[4,3-c]pyridin-3-yl)methanol; and
N-((5-(6,7-dimethoxyquinazolin-4-yl)-4,5,6,7-tetrahydro-1H-pyrazolo[4,3-c]pyridin-3-yl)methyl)-N-ethylethanamine.

84. A pharmaceutical composition comprising at least one chemical entity of claim 1 and a pharmaceutically acceptable-carrier.

85. A method of inhibiting PDE10 enzyme in a patient in need thereof comprising administering to said patient an effective amount of at least one chemical entity chosen from compounds of Formulas (I) and (II): 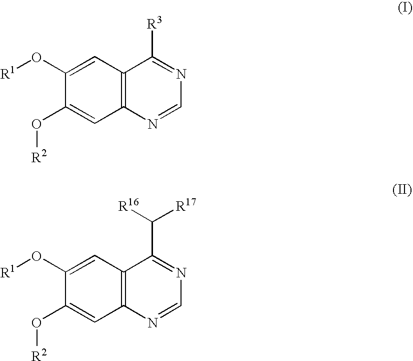
and individual stereoisomers, mixtures of stereoisomers, pharmaceutically acceptable solvates, and pharmaceutically acceptable salts thereof, wherein:
R¹ is chosen from H, alkyl having 1 to 4 carbon atoms, and alkyl having 1 to 4 carbon atoms substituted by at least one halogen;
R² is chosen from H, alkyl having 1 to 4 carbon atoms, and alkyl having 1 to 4 carbon atoms substituted by at least one halogen;
R³ is chosen from: 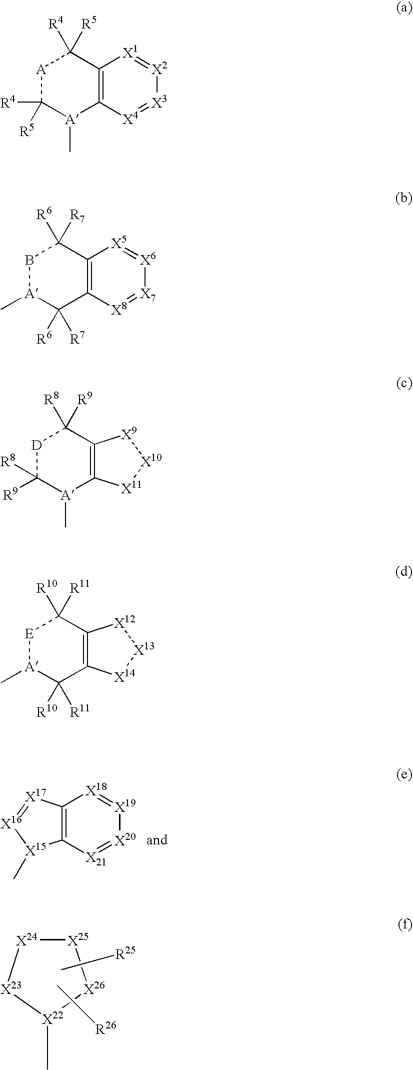
A′ is chosen from N and CH;
----A---- is chosen from a double bond, —CR⁴R⁵—, ═CR⁴—, —CR⁴═, —CR⁴R⁵—CR⁴R⁵—, ═CR⁴—CR⁴R⁵—, —CR⁴R⁵—CR⁴═, —CR⁴═CR⁵—, ═CR⁴—CR⁴═, —CR⁴R⁵—CR⁴R⁵—CR⁴R⁵—, ═CR⁴—CR⁴R⁵—CR⁴R⁵—, —CR⁴═CR⁴—CR⁴R⁵—, —CR⁴R⁵—CR⁴═CR⁴—, —CR⁴R⁵—CR⁴R⁵—CR⁴═, ═CR⁴—CR⁴═CR⁴—, —CR⁴═CR⁴—CR⁴═, and ═CR⁴—CR⁴R⁵—CR⁴═;
----B---- is chosen from a single bond, —CR⁶R⁷—, —CR⁶═, —CR⁶R⁷—CR⁶R⁷—, —CR⁶R⁷—CR⁶═, —CR⁶═CR⁷—, —CR⁶R⁷—CR⁶R⁷—, —CR⁶R⁷—, —CR⁶═CR⁶—CR⁶R⁷—, —CR⁶R⁷—CR⁶═CR⁶—, —CR⁶R⁷—CR⁶R⁷—CR⁶═, and —CR⁶═CR⁶—CR⁶═;
----D---- is chosen from —CR⁸R⁹—, ═CR⁸—, —CR⁸═, —CR⁸R⁹—CR⁸R⁹—, ═CR⁸—CR⁸R⁹—, —CR⁸R⁹—CR⁸═, —CR⁸═CR⁹—, ═CR⁸—CR⁸═, —CR⁸R⁹—CR⁸R⁹—CR⁸R⁹—, ═CR⁸—CR⁸R⁹—CR⁸R⁹—, —CR⁸═CR⁸—CR⁸R⁹—, —CR⁸R⁹—CR⁸═CR⁸—, —CR⁸R⁹—CR⁸R⁹—CR⁸═, ═CR⁸—CR⁸═CR⁸—, —CR⁸═CR⁸—CR⁸═, and ═CR⁸—CR⁸R⁹—CR⁸═;
----E---- is chosen from —CR¹⁰R¹¹—, —CR¹⁰═, —CR¹⁰R¹¹—CR¹⁰R¹¹—, —CR¹⁰R¹¹—CR¹⁰═, —CR¹⁰═CR¹¹—, —CR¹⁰R¹¹—CR¹⁰R¹¹—CR¹⁰R¹¹—, —CR¹⁰═CR¹⁰—CR¹⁰R¹¹—, —CR¹⁰R¹¹—CR¹⁰═CR¹⁰—, —CR¹⁰R¹¹—CR¹⁰R¹¹—CR¹⁰═, and —CR¹⁰═CR¹⁰—CR¹⁰═;
the dotted lines in the 5-membered ring of formula (c) independently represent a single bond or a double bond; with the proviso there is at least one double bond between X⁹ and X¹⁰ or X¹⁰ and X¹¹;
the dotted lines in the 5-membered ring of formula (d) independently represent a single bond or a double bond; with the proviso there is at least one double bond between X¹² and X¹³ or X¹³ and X¹⁴;
the dotted lines in formula (f) independently represent a single bond or a double bond, with the proviso that when two double bonds are present, they are not adjacent to each other;
X¹, X², X³, X⁴, X⁵, X⁶, X⁷, X⁸, X¹⁸, X¹⁹, X²⁰ and X²¹ are each independently chosen from N and CR¹², and wherein two adjacent X¹-X⁴, X⁵-X⁸, and X¹⁸-X²¹ groups can each be CR¹² in which the two R¹² groups taken together form a fused ring structure chosen from methylenedioxy, ethylenedioxy group, difluoromethylenedioxy, and tetrafluoroethylenedioxy;
X⁹, X¹⁰, X¹¹, X¹², X¹³, and X¹⁴ are each independently chosen from S, O, N, NR¹², C(R¹²)₂, and CR¹²;
X¹⁵, X¹⁶ and X¹⁷ are each independently chosen from N and CR¹² wherein at least two of X¹⁵, X¹⁶ and X¹⁷ are not CR¹²;
X²² is chosen from N, C and CR¹² and X²³, X²⁴, X²⁵, and X²⁶ are each independently chosen from O, S, N, NR¹², C, CHR¹², C(R¹²)₂, and CR¹²; wherein at least two of X²², X²³, X²⁴, X²⁵, and X²⁶ are not chosen from C, CHR¹² and CR¹²;
R⁴, R⁵, R⁶, R⁷, R⁸, R⁹, R¹⁰, and R¹¹ are independently chosen from
absent,
H,
carboxy,
alkyl having 1 to 8,
substituted alkyl having 1 to 8 carbon atoms and substituted with at least one group chosen from halogen, C₁₋₄-alkyl, C₁₋₄-alkoxy, and oxo,
cycloalkyl having 3 to 12 carbon atoms,
substituted cycloalkyl having 3 to 12 carbon atoms and substituted with at least one group chosen from halogen, C₁₋₄-alkyl, C₁₋₄-alkoxy, and oxo,
cycloalkylalkyl having 4 to 12 carbon atoms, and
substituted cycloalkylalkyl having 4 to 12 carbon atoms and substituted with at least one group chosen from halogen, C₁₋₄-alkyl, C₁₋₄-alkoxy, and oxo, or
R⁴ and R⁵ together form a cycloalkyl group chosen from 3 to 8 membered Spiro cycloalkyl and 3 to 8 membered fused cycloalkyl, or
R⁶ and R⁷ together form a cycloalkyl group chosen from 3 to 8 membered spiro cycloalkyl and 3 to 8 membered fused cycloalkyl, or
R⁸ and R⁹ together form a cycloalkyl group chosen from 3 to 8 membered spiro cycloalkyl and 3 to 8 membered fused cycloalkyl, or
R¹⁰ and R¹¹ together form a cycloalkyl group chosen from 3 to 8 membered spiro cycloalkyl and 3 to 8 membered fused cycloalkyl, or
one or more of R⁴ and R⁵ and the carbon atom to which they are attached form a C(═O) group, or
one or more of R⁶ and R⁷ and the carbon atom to which they are attached form a C(═O) group, or
one or more of R⁸ and R⁹ and the carbon atom to which they are attached form a C(═O) group, or
one or more of R¹⁰ and R¹¹ and the carbon atom to which they are attached, in each case form a C(═O) group,
R¹² is chosen from
H,
alkyl having up to 12 carbon atoms,
substituted alkyl having up to 12 carbon atoms and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₃₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from —CH═CH— and —C≡C—;
cycloalkyl having 3 to 12 carbon atoms,
substituted cycloalkyl having 3 to 12 carbon atoms and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄ alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from —CH═CH— and —C≡C—;
cycloalkylalkyl having up to 12 carbon atoms,
substituted cycloalkylalkyl having up to 12 carbon atoms and substituted and substituted by at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, C₁₋₄-alkylthio, C₁₋₄-alkylsulphinyl, C₁₋₄-alkylsulphonyl, —SO₂NHR¹³, —NHSO₂R¹³, —NR¹³COR¹³, —CONHR¹³, —NHCONHR¹³, —OCONHR¹³, —NHCOOR¹³, —SCONHR¹³, —SCSNHR¹³, and —NHCSNHR¹³ and wherein optionally one or more —CH₂CH₂— groups is replaced in each case by a group chosen from —CH═CH— and —C≡C—;
heterocyclyl,
heterocyclyl substituted with at least one group chosen from halogen, C₆₋₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
heteroaryl,
heteroaryl substituted with at least one group chosen from halogen, C₆₋₁₄-aryl-C₁₋₄-alkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
aryl having 6 to 14 carbon atoms,
substituted aryl having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
arylalkyl having 7 to 16 carbon atoms,
substituted arylalkyl having 7 to 16 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
heteroarylalkyl wherein the heteroaryl portion has 5 to 10 ring atoms in which at least 1 ring atom is a heteroatom and the alkyl portion has 1 to 3 carbon atoms,
substituted heteroarylalkyl wherein the heteroaryl portion has 5 to 10 ring atoms in which at least 1 ring atom is a heteroatom and the alkyl portion has 1 to 3 carbon atoms and wherein the heteroaryl portion is substituted by at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
aryloxy having 6 to 14 carbon atoms,
substituted aryloxy having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy;
heteroaryloxy having 5 to 10 ring atoms in which at least 1 ring atom is a heteroatom,
substituted heteroaryloxy having 5 to 10 ring atoms in which at least 1 ring atom is a heteroatom, and substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
halogen, hydroxy, C₁₋₄-alkoxy, C₁₋₄-alkoxy-C₁₋₄-alkoxy, C₄₋₁₂-cycloalkylalkyloxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹³, —COOR¹³, —OCOR¹³, —C₁₋₄-alkylthio, C₁₋₄-alkylsulphinyl, C₁₋₄-alkylsulphonyl, —SO₂NHR¹⁹, —SO₂NR¹⁸R¹⁹, —SO₂R²⁰, —NHSO₂R¹³, —NR¹³COR¹³, —CONHR¹³, —CONR¹³R¹⁹, —NHCONHR¹³, —OCONHR³, —NHCOOR¹³, —SCONHR¹³, —SCSNHR¹³, and —NHCSNHR¹³;
R¹³ is chosen from
H,
alkyl having 1 to 8 carbon atoms,
substituted alkyl having 1 to 8 carbon atoms and substituted with at least one group chosen from halogen, C₁₋₄-alkyl, C₁₋₄-alkoxy, and oxo,
cycloalkyl having 3 to 12 carbon atoms,
substituted cycloalkyl having 3 to 12 carbon atoms and substituted with at least one group chosen from halogen, C₁₋₄-alkyl, C₁₋₄-alkoxy, and oxo;
cycloalkylalkyl having 4 to 12 carbon atoms, and
substituted cycloalkylalkyl having 4 to 12 carbon atoms and substituted with at least one group chosen from halogen, C₁₋₄-alkyl, C₁₋₄-alkoxy, and oxo;
R¹⁶ is chosen from
aryl having 6 to 14 carbon atoms,
substituted aryl having 6 to 14 carbon atoms and substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-hydroxyalkoxy, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy,
heteroaryl having 5 to 10 ring atoms in which at least 1 ring atom is a heteroatom,
substituted heteroaryl having 5 to 10 ring atoms in which at least 1 ring atom is a heteroatom and substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl, heterocyclyl,
substituted heterocyclyl substituted with at least one group chosen from halogen, C₆₋₁₄ aryl, C₇₋₁₆ arylalkyl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl,
carbocyclic, and
substituted carbocyclic substituted with at least one group chosen from halogen, C₁₋₄ alkyl, halogenated C₁₋₄ alkyl, hydroxy, C₁₋₄-alkoxy, halogenated C₁₋₄ alkoxy, nitro, methylenedioxy, ethylenedioxy, amino, C₁₋₄ alkylamino, di-C₁₋₄-alkylamino, C₁₋₄-hydroxyalkyl, C₂₋₄-alkoxycarbonyl, C₂₋₄-acyl, carboxy, cyano, carboxamide, C₂₋₄-acyl, C₂₋₄-alkoxycarbonyl, C₁₋₄-alkylthio, C₁₋₄-alkylsulphinyl, C₁₋₄-alkylsulphonyl, and phenoxy;
R¹⁷ is chosen from
alkyl having 1 to 12 carbon atoms,
substituted alkyl having 1 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸ wherein optionally one or more —CH₂— groups is, in each case independently, replaced by —O—, —S—, or —NH— and wherein optionally one or more —CH₂CH₂— groups is replaced in each case by —CH═CH— or —C≡C—,
cycloalkyl having 3 to 12 carbon atoms,
substituted cycloalkyl having 3 to 12 carbon atoms and substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR⁸, —SCSNHR¹⁸, and —NHCSNHR⁸, wherein optionally one or more —CH₂— groups is, in each case independently, replaced by —O—, —S—, or —NH— and wherein optionally one or more —CH₂CH₂— groups is replaced in each case by —CH═CH— or —C≡C—,
cycloalkylalkyl,
substituted cycloalkylalkyl substituted with at least one group chosen from halogen, hydroxy, C₁₋₄-alkoxy, halogenated C₁₋₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and —NHCSNHR¹⁸ wherein optionally one or more —CH₂— groups is, in each case independently, replaced by —O—, —S—, or —NH— and wherein optionally one or more —CH₂CH₂— groups is replaced in each case by —CH═CH— or —C≡C—,
halogen, hydroxy, C₁₋₄-alkoxy, halogenated C₁₋₁₄ alkoxy, nitro, cyano, carboxy, amino, C₁₋₄ alkylamino, di-C₁₋₄-alkylamino, C₁₋₄-hydroxyalkyl, C₂₋₄-hydroxyalkoxy, —COR¹⁸, —COOR¹⁸, —OCOR¹⁸, C₁₋₄-alkylthio, C₁₋₄-alkylsulphinyl, C₁₋₄-alkylsulphonyl, —SO₂NHR¹⁸, —NHSO₂R¹⁸, —NR¹⁸COR¹⁸, —CONHR¹⁸, —NHCONHR¹⁸, —OCONHR¹⁸, —NHCOOR¹⁸, —SCONHR¹⁸, —SCSNHR¹⁸, and NHCSNHR¹⁸;
R¹⁸ is chosen from
H,
alkyl having 1 to 8 carbon atoms, and
substituted alkyl having 1 to 8 carbon atoms substituted with at least one group chosen from halogen, C₁₋₄-alkyl, C₁₋₄-alkoxy, and oxo;
R¹⁹ is chosen from
H,
alkyl having 1 to 8 carbon atoms,
substituted alkyl having 1 to 8 carbon atoms and substituted with at least one group chosen from halogen, C₁₋₄-alkyl, C₁₋₄-alkoxy, and oxo,
cycloalkyl having 3 to 10 carbon atoms,
substituted cycloalkyl having 3 to 10 carbon atoms and substituted with at least one group chosen from halogen, C₁₋₄-alkyl, C₁₋₄-alkoxy, and oxo,
cycloalkylalkyl having 4 to 12 carbon atoms,
substituted cycloalkylalkyl having 4 to 12 carbon atoms and substituted with at least one group chosen from halogen, C₁₋₄-alkyl, C₁₋₄-alkoxy, and oxo,
heteroaryl,
heteroaryl substituted with at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
heterocyclyl, and
heterocyclyl substituted with at least one group chosen from halogen, C₆₋₁₄ aryl, C₁₋₄ alkyl, halogenated C₁₋₄ alkyl, hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
R²⁰ is chosen from
heterocyclyl, and
heterocyclyl substituted by at least one group chosen from halogen, C₆₋₁₄-aryl-C₁₋₄-alkyl (e.g., benzyl), C₁₋₄ alkyl, halogenated C₁₋₄ alkyl (e.g., trifluoromethyl), hydroxy, C₁₋₄-alkoxy, halogenated C₁₋₄ alkoxy, nitro, oxo, amino, C₁₋₄-alkylamino, di-C₁₋₄-alkylamino, carboxy, cyano, carboxamide, C₂₋₄-alkoxycarbonyl, C₂₋₄-acyl, C₁₋₄-alkylthio, C₁₋₄-alkylsulphinyl, and C₁₋₄-alkylsulphonyl;
R²⁵ and R²⁶ are independently chosen from
H,
carboxy,
alkyl having 1 to 8 carbon atoms,
substituted alkyl having 1 to 8 carbon atoms and substituted with at least one group chosen from halogen, C₁₋₄-alkyl, C₁₋₄-alkoxy, and oxo,
cycloalkyl having 3 to 12 carbon atoms,
substituted cycloalkyl having 3 to 12 carbon atoms and substituted with at least one group chosen from halogen, C₁₋₄-alkyl, C₁₋₄-alkoxy, and oxo,
cycloalkylalkyl having 4 to 12 carbon atoms,
cycloalkylalkyl having 4 to 12 carbon atoms substituted with at least one group chosen from halogen, C₁₋₄-alkyl, C₁₋₄-alkoxy, and oxo, or
R²⁵ and R²⁶ together form a cycloalkyl group, spiro or fused, having 3 to 8 carbon atoms, or
R²⁵ and R²⁶ and the carbon atom to which they are attached form a C(═O) group.

86-89. (canceled)

90. A method according to claim 85, wherein said method treats cognition impairment or decline in said patient

91. A method according to claim 85, wherein said patient is suffering from psychoses.

92. A method according to claim 91, wherein said psychoses is chosen from schizophrenia and bipolar disorder.

93-105. (canceled)
